SEQUENCE LISTING (42 sequences)

SEQ ID NO: 1 - DNA, 2012 bp, Loxodonta africana
  source 1..2012: mol_type = genomic DNA
actctacctctcattcttttttcgccgaactctacccctcctctggtaaccctgttcattgccgaggctggcccccggcaactggcgcccgaacagggacctgaaggacgaaacttcccctttgtggatacccgacagactcatcagacatgcaagacccaaaaaccaccaaaattcctccaacacttcccccctcactgctcacaaacctaacacctcccacgactcttctgagaaatctgtctcttgaaaatgctccatctacacccccaccttcgcctgtctctagttctccctcctcttcatcttctactccaccccttctcactgactttatacaaccctctacaaacctcaacaactcttcatctcgagacaaagtgcggtggagaagccggagacatactccctattcaagaacccatcaaaccattgcggctgagcccccaacttggggccaactcaaaaaacttactcatctggctcacatcttaaccgtggatcaacatattcccgtaaatgcgggtactctttttgtggcaatgctcgctatcatttcctgtcaggtatgtgttgttatggctgatatgcattgggcttatgtcccaaaacctcctatatttcaccccgtcacctgggaagatccttccccctctgtatttactaataattctgcattactggggggcgaaaccatattttggaaccctcagggctttaattctaattattcctattctggtatgtcagataatcctcctatttgtattcaattaagaaatttgcgaaaaactatccctggctgtattccttttggcttcaaatcaatgattactgactcacctaatggaaatcaaaaccaacagccttacagactattatgggctctacgcctcatccttccagaaaaaactgactttcttaaaggtatcccaaaagtacacactcctctttttcctacctgtgataagtcgcctcctgaggattctaagagcaatcaagattgggctatgattgacccttcaaaaggatttcctatttggagaaattgtatgtataattcacaagttgtatatgctttgcctaaaatgtctgcacgtcttattgattgtagtattttaaatcctgaagatgattatcgtctatttttacaatcagctcgatatcgttttaattggcaagataccttagtccctctcccacaaaaagttaatcgttggcatataaatggatgggttccccctattctttctaccttcacgggtaacatacatccatctttgtggagactggcaactgctgccggcaatgtcactttgtcccgtccttttaataatgagagttttgatattcaagcttgtgtacctgctccatatgtgttattaattagccgcaacaatcactctagtattgttatagaaaacaagaaaaaacattatgttagttcttgtaaaaattgtatactgaccagttgtataaatcctgcaattcctgttgaaagtatgatgattttacaacaaccaaaatatattatgattcctgtacatttacaagaagactggtatgatgattcaggcctagaagccttaaaacaagcttctgctattttatctcgagccaaacggtttgtagcagctttaatactggggatttctgctctcatagctttagcaacttctctttcactttctgctatggcgttaacacaacagattcatactgcaaactatgtaaataatttaagtaaaaatattactttagccttagccacacaagaagccattgatagaaaattacaacaagaagttaatgcccttgaagaagcaattctatatataggacaagaaatcactaatctcaaagtgcgacttactttgagatgccattcatcttccgaatcttaaatcaagctatccaaaaggtggcttcagatcttcatctggcgaaattaaaaaataagaaagggggagatgctgggagccaggctgcaagggcggcccctctgcacccac

SEQ ID NO: 2 - DNA, 2019 bp, Mesocricetus auratus
  source 1..2019: mol_type = genomic DNA
atgcttgtgtgttcccacagaatgaaagctcaccgatctggattccagacagacttatcagaccctaccagcaggagactctcacggaaaacaaagaggtggagaaaccgcgagctacagacaatggggaagcagctgacgagtctgaccataacaaacaaagaaggtgaagtcacctcagaaggtgaacttagctgcaatctgccaacacagactcaactcaagaacctacaaggagaggctcttaccgtacgggctcaaacaaaaactcctaagactcctgtagcaatttttcttgcttttcttgctatcttatcaatgaattccgcggtaaatggagaaagttactgggcatacattcccaagcctccacttttacatccggtggtttggggaaatacagagctcattaaaattatgactaatgttactgatgtgctagggggaactcctgattttcatatgggaattaattcatctgggtatataaattatgaaggtagagcagattccttaccaatctgtatagcacttgatggtatgcctccagctggttgtttgcctgtcagctatagaactttcctcactgatactcctgataataaacctgttacaagtaggagtccagaacagcggcatatgtggcaactacaaattgctactttgggacactctaattttaatgagactatggtggtaaatcagccgctgccttctagattgccacattgcatggtaaaatacaagaaagatagcttatgggagggagatgaagatcagatgcctaattggcttcattgtggatttcctcaagctggaacaaggttttctcctttgacttcaggagaaattatctgggatttctctgtaccatcacctgatcatgactattctggctaccttaaggatcattctgctcaatttggaggatacactcctccaaaaaatgagcccataaggagatggtattcagcagggtgggtgtcacctgtatggtatttgcaaaaaccggatgggacatatgggactcctcagccagatctattcaggttagttgcagcagccaatgttataatattaaagaaaccaaaggctgccaagtcagagcctgttcctgccactgcttgtctctcttacccatatgccttaatgtttggacgtaacaaattggtgcatatcaagaagaaggggactcatttttttgttaaatgtcgatcctgcatattaactaattgtgtacaatcaggttatgataaatccttttcaactgtattgattgttaaaaggcctgcttatgtgatgctgccaatagatttgggtaatgatacttgctacgataatttggctataaacttgtttaacaaaattaatgaattgattaggccaaaaggatttgttgcatcattaattttaggaatcacagcactgatagacattttgacttcttttacagtttctacaacagcattagtaaaggaaatgcagacagcttcctttgtagatgacatgcataggaatgtcacctttgccttgtcagaacagaggttaatagatctgagattagaaactagattaaatgctttagaagaggcaataatagaaatgggccaagacatacaaaatcttaagacacaattagcaactaaatgccatgcagagtttaagtatatctgtgttacacctcttccatacaactcttcctttaattggaacaagactaaggcacatttattaggagtatggaaggataccaatttaacctatgatatcaaggcattgcaggagaaaattacagtcatgagtcagaaaaagatagatacttgggacatttcaggcctagcatcagaattttcagaaaacctaagatccctgaatccattaagctggactcagtattttgttttcctaggaattggagctgctattttatttacagtgtgcttggttttcctattcttttcagaataccacttcactccatcggcaccctccagcgtgatttccagctgttcctcttaa

SEQ ID NO: 3 - DNA, 2064 bp, synthetic construct
  source 1..2064: mol_type = other DNA; note = BoRV_CH15_env Bovine retrovirus beta-like env NCBI accession KU720628
atggtcttgttcttttgcagaatggaaaacaacaatggctcccggcgtgtaggattcaagtacggcggaccaccggccaagaaacaattgctacagcctccgcggtttgaccccactgttacagttcctgaaatagaaataccctatcaggctttttgtcgtatgacgttaggagaacggcgccgtaaacggcctgtgacggtgcctttgcctacatggggtcagataaaaaagctttcgcaagaggcctttcgcactgttatgggaacgggaaaagttcctacacctgaaaatctatttcttgctatgtgtgctattttgactgtaacctcctctcaggcagcttctctccctgttcctactccagaagctgacgtaagttatacctattgggcgtatatcccaaatccccctctgttggaagcaacggtttggggagtttcagatattttgatatatactactcctcatgtgttgtcacctccttggaagaatctaacatatttgaataaagatgatggccgcttgtttaatttttctcagcgtttaacccaaggaactcctatctgtttaacaaaggaagctcgtgacccctgtttggtgatggattggcagagttggcttttaccagggagaaatgaaagttcaattagggcacgtttacagcgattgacgacttggtcaattaataatagttacaataatattagcttgcaggaaactgcatcagtgcctttttgtacgcccttcacgtatataagtttgaaatataaaccatttatttggtctgaatgtaccagtgaatgggccaaaagaactggagattatataaattggggtccctttggtatgttttttaatgattgttcggaatggaataataaaacttgtcttttctttaacactaccagactcctgatgcccatgaatcaaacacttcaaggatataaattgatgtggtggggagatggtggcgtcagtaacccgagatggactcacgtgcagtatcctgataattatcaatctcatctgtggaaagtggcagcggcattgcaaccagtgtacaaggctcaagggcaattcctcggcacttctcgttccttctcaacttatagtttctttaatatcacccaatataatcttactgcttgtattccattaccttatttgtttttagtagggaattttatgtataatgaaggatttatacaatgtaacaattgtgcgttatactcatgtcttaatgcctccattgatgcccctgctattatgatattacaacaacgaacacatttgtggttgcccgtgaaaatggaacagccatgggcatctagccccttagataaaataataatacaagtattgaagaaggttttgcatcgttcgaaaagatttattggagccgtaattgctacggttctgagtttgattgctattgttaccgttgctaccgtgtctagtgtggcattaagcacagcaatacaaaataaacactttttggaaacatggcatcaggattcttttcaattttggacagaacaaactcagatagatgcacgatttcaacagcagatagatatattaaagcagtctgtacaatggttagggagaaaaatgattcaattagaaaagcaaatatcactaaaatgccactggaatagtagcaccttttgtattacccctatattgtataacaaaacggaatcagactgggttgttatacaacatgccttagaaggaaatatgactgcggtacaattaatagacgatttgcaaaaagaagtaaatcaagaatttgggaaaccttattcgaatgactactcaggagaaattgctgacgatatatttaaacatttggagaaacttaatccttttacatggggacaaagtcttatacattcggctggaagtttaggagtgtctattttaatattaattataagtttgattttgatatatcggtgctgcatagtgcctttacaactgtctttaacgcaaatgtggacaaaaatgttgatttctaaaaagaaaaaagggggaattgaggacatcggcaagagttaa

SEQ ID NO: 4 - DNA, 1731 bp, Bos taurus
  source 1..1731: mol_type = genomic DNA
atgccgaagcgccacgccggctgctggaaaggttggtacgcacgccggagacgatctctagcaaaccagatgaggagactgacgattcaagagcatgatgaattaccatctcggcaacaacttcagacattgatgcgagaggcacagaatagtgttgctgtgcagggcgatccgatatcgcctttaagcttcctgataaccttattatttatcttaaatcagattaatactgcacattctgaagaatattcaagtgcttactgggcttattttcccaaccctccccttctccaaccggtgggctgggagggtcggtctattcccatatacactaatgatactgtggctttgggtggtttttcagataaacatattattcctaatcaagttaatttctcttatcatggagtgtttgatcgcttacctatttgtctgtctagatattttaattcaacaggatgtctttttgttggggagtcaggatatgctgattatgacaatggttgtagcctgtatattcctggagtaatgaaagagtctggaattcctcaacgtcgtaatggaactggtcctttagatatccctttctgcgactttggaacaaggggaagagtcactgctgcaccatggaaactgtgtcgagatggaattgctacggtttataacatatttgggacaaatgagtcattgtgggactggtctccagactcggcccgaaaccctggagctcaggataataggaaatttgcatcccgtatttggaacttgggcggctctaaagtgttccaaacagaaatctggaaactagcggccgcccttggatgtgagggttcctaccttcaggtgggatcgaaagtgagacacggttatttgtggaatctcaccatgagataccctcgtgcatgtgtgcctcacccttatgctttactagtaggatctgtaaatatacaacctgggttacgaaattataatgtaacttgtaacaattgtactttgactaactgtattaggggaattactaatcaaactagggttctagtcttaagacagcctgcttttgttatggttcctgtaaagattaatgggtcttggtatgatgaaaggggacttgaactttggagagaagtagagggtgctttaatgctctatcgcaggggaatagggttaataattctgggatttgtagctttggtaactcttactgcttcctggattactgcagccctgtccttggcacaatcagtgcaaactgcaacttttgttaataatctggcacaaaatgcatccgttgccctggggactcaagaagatattgacaaaaagctagaggatcgattgaatggcctttatgatgttgtaaaatttttaggtgaagaggttcaaagtataaagttgagattacgagtacaatgtcatgctgattttcaatggatctgtgttactcccaaaaaatataatggtactataactgcttgggataaggtgaaagctcatttagaaggtatttggcataatgaaaatatttccttagatttgctgcatctacatcaggaaataatgaacattgaaaatgcacccagacttgatttggatgttgcaaaaagagctgagtcttttgttaaagaactttttcgacatgtacctactgtcaatagtatttggtacttgggagcagccataggaggactattcttaataattttaacttttatttcttgcaccttgtataat

SEQ ID NO: 5 - DNA, 2240 bp, Bos taurus
  source 1..2240: mol_type = genomic DNA
atgacccctcaggctgaacaaacactgaaggttactggaactcccatgactccagataagctgtttctggcaatgctagctgttctctcttgctcctctggggtaagtgtgaaatatctttactgggcatacctacctaatccacccttgttgtccatactggattgggtcaacaaggctcctgtggtattcaccaatgacagcctgcacttcccagctccatggtcaacacaaagaccaattcatgaagaagatgaaggaagaaaaataaatatttctattggattcaaaaccttacccatatgtttcggatttcaccctttatgcataactatattcccccaatggtgggcttactcagcacacaatggttctgcctatcgtataggaatgtttgcaaccacatccttcctgctaaatggctctgagaggcattatcctggacaaacagttaactatattaattcactcttccagcctgaagaaccaatatgtgatgttgtatcatggagaaggaagcaatcaattcaatctctatattggtcagattgtcagggacagtttgggaaaatctctatcattcagcagaatcactttatatttaaattcattgactgggggccccacagattgtttgtgaactgctctgaggaacaggaaaagaaccataactgttcctggtataataagtccaatgtgtggggatttcataagaccaatactagcttttggactgataaagacatactgttaaattggtataatggaggattgtctccacctcggcccagaatcatagtccctactgtgggacctgagcaatggcgcgtgtggaagattcctgctgcactggagcactttgccagtgtgtatgggactgtgaaagccttaaaacctcaaaactatacctttttatacaatagtacaggttatatccaatcttgtgtgcacctcccatacatgtttattgtgggtaactttgatatggatctttctgctaaaattgttaacagtacagcatgtgccttgtatacgtgtttaaaccatactatttcatatcataatgtcaacattgcagtagtcaagcaaagatctgaattgtggcttcctgttaacctaacagaaccatggaccgactctgtatttctctcagttttgctcaaatatggcttaaggaggtcaaagtgcataataggatggattattgctggcatccttagtctaatatctattgttacagtaggaaccctatctggtatggcactacacaattctatacagaatcatgattttataactgcttggcacaaagattcccataatctctggactcaacaagctcaaatagatcagcaattgcaaacacgaattaatgaattacaaacagtggtaattaatataggagatcaagtacagcaacttactttcctaacacatatatgttgtcactggaattttacttctttctgtctgactaacatgccctataatggcactgagtatccttgggacaaagttaaagtgcattttcaggatctcatggataactcaagtttggacatcaaccttctaaaacaacaaattgctaattttcaagttaaaatacctaaggaattatatagcacccaattttatgatattttaaccaaaacattatcctctctggatcccagaacttggttttcaggaggcaacttttttatgtatgtattaattgctctgctttttgtcattgtttgtaggatacagatgtctctactcaagactctgggcctcccacagtggagtccaactagcagctgccatgcttaaactaaaaacaaaaggagaatatgttgggaggcacagtgcaaaatagccttgaaggagaaggtccttgggaaaatggcgaagggccaaaagtacccaggcattgtgtactgatagctgaaaaacatttcctgcctttggctatgcttggtgccaagatttaacaattaaacattaaagacgttgcttgagaaaatgggtcatggataaacacatgggtcgttaagaatgcgctggtccttgaagtatcttttactgattatatcttagccttgtacgtgttctgctggccatatgctctaagctctttgttccttgctcctataaaaaggcttgactgcagaaataaagttgtcagtccttgcagagactgtccaagtgttgttctttcaggttcgccgtttccaagtcccagggacatatctcccaca

SEQ ID NO: 6 - DNA, 2142 bp, Bos taurus
  source 1..2142: mol_type = genomic DNA
atgtcacagccccccaggcatctggtccctccactgccacgaccacaaaaaaggaagggacctcctctacctccactccctccgccaacacgggaagcccagctgatctctagacgaatgacaccagagatcctggatgaacaggaagaggaaccacccaccagacagatgtcacaactctgtataagagatattgttctccctaattctttaccccacaggaaatcaccgggatgtcctacgcgggccactcaacaagcctctatacctacctggggacagattaagacgctttgtcaccaggcacaagggatagcttccctgcagggctctccagtctctccggagagagtgtttattgctatgcttgccttactatcttgccaggtaagcgcctcctctcccgctccagaaaagtattgggcatacttcccagatcctccaaccttccaagtagttacttggaacaatgcccctatacgggtccacacaaaccagcctcatttattgggaggacattatactttctatacagaggaggaataccctattaatttcaattatacctttaggggattaacagatgatctccctgtttgttttaacttcccttttgatcatacaggaagctatattactcctaccaaagaaggatgtattggagcctctgaaaaagcaattataacagattctccatccttaagatatcggtcagtttgggtattactggctcgtatgcctgggatccttgacccctataaatccctgcattttaaatctccaccaaaatatccaaattgtcataatgctgccccttcagatgccatatggaaaactatagatggtcattcaggatatccgatttggaaatcttgtacttataattctagaattgactacagaataccaggaggaaattataccattcaggactggagcaacccggatccaggtcaggatccaatgattgatgatgcatttaaagggagatttgacaattggaaagaatattcagttccttggccattacgcgccactagatggcatcgtaatcaatttgttcctcctatgctgtcttatacggctaagggcagaacttattggcaaccagaaatttggaaggcccttaccgctactgcccctattaccttaacccggccagaaaatatttctacttattctattttagcttgtctaccctcgccttatgtttttctcttcactaatgattccaagagacttaatatacacatgaattattcaggtggacctaatatagtaacttgtgaacaatgtatgctctcatcttgtttggcccctcaatataatgtttgctcttttgtggtgttgcaacgcccaccctatcttatggtgcctgtaactataactacacattggtatgataactatggtcttgctgtaatacaacaagtgcgggatttaatgcgatcacgacggttagcgggcttacttttactgggaatagcaactttaataactggaattacttctgtttatatggcagcagtttcattgactgaacaagtacacactgctcaatatgttgatgctatgtccaaaaatgtttctttagcattggcaacagaggaagctatattcaggaagctggagatgaggatagatgccctagaggaagcaatattgcatattggaaatgaattgcaggctttaaaagtgagattggcactgtcctgtcatgccgactatcggtggatttgtgtaacacccctgaaagtaaatgagacagattatgactggcaaaaaattaaaaatcatattttaggtgtctggaacagctccgacattagtttagatttaaggaaacttcacagtcaaataacaaccatagaacgctctcacttagattttactgcctcaggagtagcaagtgacttcttccatacaatctctaactttgtttcaggaaagtactttctgtctgctatttttaactatgctgctgtggccgctataattctgcttatcatattcattcttccttgtattgtcagaattttccgacagagcattcagaagctcgagactgaattgcatacgggtttcttaagaaataaaaatgggggagatgccgggacccagcacgggagttcccacccatga

SEQ ID NO: 7 - DNA, 2413 bp, Bubalus bubalis
  source 1..2413: mol_type = genomic DNA
ttaaaaatccaaattcaaaaattaaaagaaggggagtttaagtatagctctccccatcaaatcttgcaacatgctcttttcgtaataaatattttaaatgccgattcggccggaattactgcaatgcttcgccattggtgccctgagcagctgaatgcaaaaccattagttaaatggaaggacctcctttccggacaatggaaaggtcccgacccattgttaaccagtggtcgaggatatgcttgcatttttccacaggacgcagattctccaatttgggttccagacagattgattcgccatgtctcagcccctcggataccggattccccagctgccgcgaccccgaaaaaggaagggacctcctctgccttcattccccccgccagcacgggaaacgccgccggcgtcgggacaactgacatcggagatcccggatgagcaaggaacggatccacccaccaagcagatgtctcaacttcatatacaggatattgctcctcccgatcctttgcctcacaaaagggtgtcaggccgcctgactcaggcgacccggaatgcctccatacctacttggggacaaataaaaacactctgccaccaggcacggggaatagcttccttacagggatctccagcttctcctgagaaactgtttattgccatgcttgctttgctttcctgtcaggtaagcgcctcccctattccaaccaaatattgggcgtatctcccagatcctccaactttccaagttgttacttgggataatgagcctatacgggtcaacacagaccaaccccagctcttgggaggatcctatacttcttacactagagataaatatcctattaacttcaactataccttcaggggattaacaggtgatttccctgtttgctttaacttccccaatgaccttagaagctttattacccccaccaaagaaggatgtgttggggcctctaaaaaaataattataacagattcctggtctttacgtcatcgatcagcttgggtattattggctcgtatgcccgggatccttgacccctatgtaaccctgcattccaaatctccaccaaaatatccgaattgttataaggttgctccttcagagaaagtatggaacaccatagacagtcgtacagggtatccgacttggacatcttgtacttatgattcaaggattgattataggataccaggaggtggagattatactattcaggactggagcaacccgaatccaggtgaggatccattggccaatgatacatttgagggcagattcgagaattgggataggattcctcttccttggccaacacatgccactagaaggcatcgtaatcaatttgttcctcccatgctgtcttatacaacgaagaaacagacctattggcaacctgatatttggagagcccttgctgctactgctcctgtctccttgtaccgcccagatagcaattctacttattctgttttagcttgtctaccctccccttatgtttttctttttactaatgactccaagaagcttaatgtacgcatgaattattcaggtggacctaatatagtaacttgtgaacaatgtatgctttcatcttgtttgacccctcaatataatgtttgctcttttgtggtgttacaacgcccaccctatctcatgatacctgtgacagtgactagccactggtatgacaattacggcctcgccgtgttacaacaactacgagatttaatgcgacaacggcgatttgtgggcttacttattttaggaatatcggctttgatagcagcaattacttctgttactgcggcagcaatatcattgagtcaacaagtgcatactgcccaatatgttgataccatgtctaaaaatgtctctttaactctagcaacacaggaagttatagatagaaaattggagatgagagtagatgccttagaagaggcaataatgcatattgggactgagttacaggcgttaaaagtgaaaatggctctgtcttgccacgctgattaccggtggatatgcgtgacatctttaaaggtaaatgagacagattatgaatgggaaaaaataaaaaatcatatctcaggtgtttggaacagctctgacattggcctggacttagggaaacttcataatcaaatacagaccctggaacactctcgactggattttactgccgctggggcagctaatgacttttttcacactttctctgacttcatttcaggaaaaaacattctgtctaatatccttggatacatcgctgctggtgctttagttttgcttctcatattcattcttccttgtattgtcaggattcttcggcagaacattcagaagctcgcgactgagctgcatctggctgttttaagaaataaaaaagggggagatgctggg

SEQ ID NO: 8 - DNA, 1560 bp, Canis lupus
  source 1..1560: mol_type = genomic DNA
atgaggacaccccgcgctggctcccggagtgatggtgtcgactcctcacccctacgccggagactagccaacctaaccataactcgccaacaaaaaagaagaaggagacgtctcccatcctactgttgggcccggataagggaactgatcaatcaagcgataagtctggctgaacagacacagcctaatgagtccttacttactatccttcttactgtattagctattaccacccctcccgttggtgaagcggctgtatattgggcctatctacccgacccacctactataaggccggttacttgggatgatcctactccccaaatacacaccaatatgacagaatattttggagggagtagttctgatgacatggaggttgcaaaccattctatcaattggacggggctggcagctcaggtacccatgtgcttccaattaccactatgtgaccaaaaaccagttaatggttgcttggtagttcagccagtacctcacatagtaacatatctctcagaaatggaaaagaatgaaaaccctccatcccgacttaacataatgttagaaaggtggattgtctatgaggtcactggtgacacccctcccagtgaccctcaacaaggtaggcgccctcccgggtaccccaattgtcctcaaaacttaacctatgctatttcaggtcctatttggacagagtgccttcagaagacacctttggtcatctctcctcacggcatccctgaattttctatcacagactggtcgagaatgacaagaaaggagcctctgcgctatattgcatatccacctggtcgaccaatgcataatgtgactatacccggaggagttttttccaccgtcggagaaatgtaccacgcagaactctggaaattggtcaccgctatgggaccagccaagaggcagatggctaagagcggctacacgaacacaaaggacttcaatcttactatacaggcctgtgtccctaagccgttcctattggtggtaggatatggaacgacaaatttttcctttatgcaaaataatggaactgatatgttatttgaactaggatgtaaaaattgtgtattaaccaattgtttgcctgtgcacttgccctcacctcaaacaggtcctatcactatctttttgactatgcaaccgccttatttactattacctgttgagattgaaggcccttggtatgctaattatggttatcagtttgctgtggaattgcagctacaattaaaaagattgaaacgcttccttgggctgttgatagccggtattgctgcattggttgctctgatagctactgcagctgcagcctctgtggccttatcccagggcatacagacagcacaatacgttaacaacttagcaaaaaacgtctcttacgcgctgtctactcaggagcgtatagatcaaaaaattctcagttgtcttgatgggcttgaagaagagaaatcaggagtaaaggggatcttggagagtattttatccagctcttccatgtcacagcaagacaacaagttgagagattcacacggactctga

SEQ ID NO: 9 - DNA, 2437 bp, Equus asinus
  source 1..2437: mol_type = genomic DNA
ccagtttttcaccgccgccgcctctccggcgggaacctggactctgcccaggctggaccctggcaagtggcgcccgaacagggacccaacgggacgcttcctctcctttgtggattcctgatcgtttgatccggcatgtacagcaggcggtgccggacccttcctccgttgagaagaacggacaacagcgccctgacagagactgatgaggatcatcctcctgtgatccagatgcggtggctttcgctccgtgacgcaccctatcagcgtcttcctcctcgatcgccgccgcctccatatccgcgctctaaagccactcgagctgcgtcaattcctacatggggtcaactaaaacaccttacccaagaggcagaacacttagttcgggtgcaggggggtacaatttccccctctactctgtttctagctatgttagccttgattgcctgtcaggcaggaaagacctctgcctcccttacccaatattgggcttatgttcctaaaccccctctattccatccagtaacctggaaggaaggacagattcctgtcacgaatgaccatcctgagttattaggaggtctgtctatataccatgataaaattgacattaacaaagatttttcttttcaaggtacctctagcagtccccctatatgctttaatgtacaccctgtagttcggatgccggctccactgccagcaactaacatagcaggctgtattggaacctctattaaggggttacttacagactcccctaacggaatgtggagagatttatggtccctgcaactattactgcctggggacatgggccccagggttctgaactctgccatccaccctccaggatatcctacttgtcctggaaggacaatatccccagagaaattgcctactaatagtgactggacgtcggttggcaaacgagctggcttcccaaagtggagggagtgtatgtattcatcgaaaattgtgataaaaataccttcttttcctggtgagatatttgattggagctgtcaggacccttcaaaagattacagaaattatttacgtgaaactggtaaataccggtggtcagatcgttacatagctcctgaactggcagtggatcattggcatatgaatggctgggttccccccatattgtcggctgactctggtgaggtccagcctcacttatggcaggctgtagccgctctacacaacattatcctaaataggccagtggggtcccagtcctataaggttaaggcatgtattcctgccccttatgttttcctggctgcgcccactcacagtcgtgcaatgactattctacccaccacggacggttccatatataacattacctgtaccaattgtattctgactaattgtttaaatcctgatatagacgtggaaactgttatgatattacaacaacccccctatcttatgctacccgttcatttgtctgaaccttggtatgatgatgttggattgcttactttaaaacaggccactgatctcttacagcgcccccgccgatttatcactgctttgatattgggtatctcagccttgattacattagcaacctctctcactgtatctgctgtagcgctgagtaaacaaatacatactgcctcttatgtagacgatctttctaagaataccactcttgccctaactacccaagaaataatagacgaaaaactggaggcaaaggtaaacgctctggaagaggcaatattacagataggacaagaacttacaaatttgaaggttagactggccttgcgatgtcatgtgtcctatagatggatctgtgtgactcctcttaaggtaaatcaatccctacactcctgggagaaaattaaaaatcatattcagggaatatggaaccattcagactttagtttagacattagtaatttacatagacaaattaataatattaatcaggctgaagaatccttttctgcttccaaagtagccaccaactttttcacctctctgacctctttcgtaggggagaaaaattggagctcccttattgttaatattgctgtcaccattggtgctctaatgatttgtctttgccttttaccaatctttctcaaattaatattcaactctataagtcgggtttcggcagaattacatctgttggccttaaaaagtaaaaaagggggagatgctgggagccatggctacatcatttgatcggctgtttgacagggcccagccgattaacgctgaggggtgcagggtcgccccttggtgaagaataaccggccagcccctcacatagttctgaaacctgtgctgcttctaattgcccttcattccttttcctttcttaacctccagttttcactcctttgggtggctgcttgggaggcactacctcccaggaaaattagatatagtaagagaatgtgaccac

SEQ ID NO: 10 - DNA, 2100 bp, synthetic construct
  source 1..2100: mol_type = other DNA; note = HERV-K108_env_ NCBI Accession: AF164614
atgaacccatcagagatgcaaagaaaagcacctccgcggagacggagacatcgcaatcgagcaccgttgactcacaagatgaacaaaatggtgacgtcagaagaacagatgaagttgccatccaccaagaaggcagagccgccaacttgggcacaactaaagaagctgacgcagttagctacaaaatatctagagaacacaaaggtgacacaaaccccagagagtatgctgcttgcagccttgatgattgtatcaatggtggtaagtctccctatgcctgcaggagcagctgcagctaactatacctactgggcctatgtgcctttcccgcccttaattcgggcagtcacatggatggataatcctacagaagtatatgttaatgatagtgtatgggtacctggccccatagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcccatctgtagattcacttatcacatggtaagcgggatgtcactcaggccacgggtaaattatttacaagacttttcttatcaaagatcattaaaatttagacctaaagggaaaccttgccccaaggaaattcccaaagaatcaaaaaatacagaagttttagtttgggaagaatgtgtggccaatagtgcggtgatattacaaaacaatgaattcggaactattatagattgggcacctcgaggtcaattctaccacaattgctcaggacaaactcagtcgtgtccaagtgcacaagtgagtccagctgttgatagcgacttaacagaaagtttagacaaacataagcataaaaaattgcagtctttctacccttgggaatggggagaaaaaggaatctctaccccaagaccaaaaatagtaagtcctgtttctggtcctgaacatccagaattatggaggcttactgtggcctcacaccacattagaatttggtctggaaatcaaactttagaaacaagagatcgtaagccattttatactattgacctgaattccagtctaacagttcctttacaaagttgcgtaaagcccccttatatgctagttgtaggaaatatagttattaaaccagactcccagactataacctgtgaaaattgtagattgcttacttgcattgattcaacttttaattggcaacaccgtattctgctggtgagagcaagagagggcgtgtggatccctgtgtccatggaccgaccgtgggaggcctcgccatccgtccatattttgactgaagtattaaaaggtgttttaaatagatccaaaagattcatttttactttaattgcagtgattatgggattaattgcagtcacagctacggctgctgtagcaggagttgcattgcactcttctgttcagtcagtaaactttgttaatgattggcaaaaaaattctacaagattgtggaattcacaatctagtattgatcaaaaattggcaaatcaaattaatgatcttagacaaactgtcatttggatgggagacagactcatgagcttagaacatcgtttccagttacaatgtgactggaatacgtcagatttttgtattacaccccaaatttataatgagtctgagcatcactgggacatggttagacgccatctacagggaagagaagataatctcactttagacatttccaaattaaaagaacaaattttcgaagcatcaaaagcccatttaaatttggtgccaggaactgaggcaattgcaggagttgctgatggcctcgcaaatcttaaccctgtcacttgggttaagaccattggaagtactacgattataaatctcatattaatccttgtgtgcctgttttgtctgttgttagtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtag

SEQ ID NO: 11 - DNA, 3022 bp, synthetic construct
  source 1..3022: mol_type = other DNA; note = HERV-K113_env HERV-K113 complete genome NCBI AY037928
atgaacccatcggagatgcaaagaaaagcacctccgcggagacggagacaccgcaatcgagcaccgttgactcacaagatgaacaaaatggtgacgtcagaagaacagatgaagttgccatccaccaagaaggcagagccgccgacttgggcacaactaaagaagctgacgcagttagctacaaaatatctagagaacacaaaggtgacacaaaccccagagagtatgctgcttgcagccttgatgattgtatcaatggtggtaagtctccctatgcctgcaggagcagctgcagctaactatacctactgggcctatgtgcctttcccgcccttaattcgggcagtcacatggatggataatcctatagaaatatatgttaatgatagtgtatgggtacctggccccacagatgattgctgccctgccaaacctgaggaagaagggatgatgataaatatttccattgggtatcgttatcctcctatttgcctagggagagcaccaggatgtttaatgcctgcagtccaaaattggttggtagaagtacctactgtcagtcccatcagtagattcacttatcacatggtaagcgggatgtcactcaggccacgggtaaattatttacaagacttttcttatcaaagatcattaaaatttagacctaaagggaaaccttgccccaaggaaattcccaaagaatcaaaaaatacagaagttttagtttgggaagaatgtgtggccaatagtgcggtgatattacaaaacaatgaattcggaactcttatagattgggcacctcgaggtcaattctaccacaattgctcaggacaaactcagtcgtgtccaagtgcacaagtgagtccagctgttgatagcgacttaacagaaagtttagacaaacataagcataaaaaattgcagtctttctacccttgggaatggggagaaaaaggaatctctaccgcaagaccaaaaataataagtcctgtttctggtcctgaacatccagaattatggaggcttactgtggcctcacaccacattagaatttggtctggaaatcaaactttagaaacaagagatcgtaagccattttatactatcgacctaaattccagtctaacagttcctttacaaagttgcgtaaagcccccttatatgctagttgtaggaaatatagttattaaaccagactcccagactataacctgtgaaaattgtagattgcttacttgcattgattcaacttttaattggcaacaccgtattctgctggtgagagcaagagagggcgtgtggatccctgtgtccatggaccgaccgtgggaggcctcaccatccgtccatattttgactgaagtattaaaaggtgttttaaatagatccaaaagattcatttttactttaattgcagtgattatgggattaattgcagtcacagctacggctgctgtagcaggagttgcattgcactcttctgttcagtcagtaaactttgttaatgattggcaaaataattctacaagattgtggaattcacaatctagtattgatcaaaaattggcaaatcaaattaatgatcttagacaaactgtcatttggatgggagacaggctcatgagcttagaacatcgtttccagttacaatgtgactggaatacgtcagatttttgtattacaccccaaatttataatgagtctgagcatcactgggacatggttagatgccatctacagggaagagaagataatctcactttagacatttccaaattaaaagaacaaatttttgaagcatcaaaagcccatttaaatttggtgccaggaactgaggcaattgcaggagttgctgatggcctcgcaaatcttaacactgtcacttgggttaagaccattggaagtactacaattataaatctcatattaatccttgtgtgcctgttttgtctgttgttagtctacaggtgtacccaacagctccgacgagacagcgaccatcgagaacgggccatgatgacgatggtggttttgtcgaaaagaaaagggggaaatgtggggaaaagcaagagagatcagattgttactgtgtctgtgtagaaagaagtagacataggagactccattttgttatgtattaagaaaaattcttctgccttgagattctgttaatctatgaccttacccccaaccccgtgctctctgaaacatgtgctgtgtcaactcagagttgaatggattaagggcggtgcaggatgtgctttgttaaacagatgcttgaaggcagcatgctccttaagagtcatcaccactccctaatctcaagtacccagggacacaaaaactgcagaaggccgcagggacctctgcctaggaaagccaggtattgtccaaggtttctccccatgtgatagtctgaaatatggcctcgtgggaagggaaagacctgaccgtcccccagcccgacacccgtaaagggtctgtgctgaggaggattagtataagaggaaggaatgcctcttgcagttgagacaagaggaaggcatctgtctcctccctgtccctgggcaatggaatgtctcggtataaaacccgattgtatgctccatctactgagatagggaaaaaccgcctcagggctggaggtgggacctgcgggcagcaatactgctttgtaaagcattgagatgtttatgtgtatgcatatctaaaagcacagcacttaatcctttacattgtctatgatgccaagacctttgttcacgtgtttgtctgctgaccctctccccacaattgtcttgtgaccctgacacatccccctctttgagaaacacccacagatgatcaataaatactaagggaactcagaggctggcgggatcctccatatgctgaacgctggttccccggttccccttatttctttctctatactttgtctctgtgtctttttcttttccaaatctctcgtcccaccttacgagaaacacccacaggtgtgtaggggcaacccacccctaca

SEQ ID NO: 12 - DNA, 2097 bp, synthetic construct
  source 1..2097: mol_type = other DNA; note = HERV-K_Phoenix_env HERV-K Phoenix env Dewannieux et al 2006
atgaacccatccgaaatgcagagaaaagcaccaccaagaagaagaagacacagaaatagggcaccactgacacataaaatgaacaagatggtgacctctgaggaacagatgaagctgccaagtaccaagaaagccgagccccctacatgggctcagctgaagaaactgacacagctggccactaagtacctggaaaacaccaaagtgactcagacccccgagagcatgctgctggccgctctgatgatcgtgtccatggtggtctctctgccaatgccagctggagcagccgctgcaaattacacctattgggcttacgtgccattcccacccctgatccgggcagtcacatggatggacaaccccattgaagtgtatgtcaatgatagtgtgtgggtccctggaccaatcgacgatagatgccctgccaagccagaggaagagggaatgatgatcaacattagcatcggctaccgctatcctccaatttgcctggggcgagcacctggatgtctgatgccagccgtgcagaattggctggtggaggtcccaactgtgagtcccatctcacggtttacctatcacatggtgagcggaatgtccctgcgaccccgggtgaactacctgcaggatttcagttatcagcgatcactgaagtttcggcctaaggggaaaccctgccctaaagaaatcccaaaggagtcaaaaaacacagaagtgctggtctgggaagagtgtgtggctaatagcgcagtcattctgcagaacaatgagttcggcactatcattgattgggcccctagggggcagttttaccacaattgctccggacagacccagtcttgtccaagtgcacaggtgagcccagcagtcgactccgatctgacagaatctctggacaagcacaaacataagaaactgcagagtttctatccctgggaatggggagagaagggcatctcaactccacgccccaaaatcatttcacctgtgagcggacccgaacatcctgagctgtggcgactgaccgtggcaagccaccatattagaatctggtccggcaaccagactctggagaccagagacaggaagcccttctacacagtggatctgaatagctccctgactgtgcctctgcagtcttgcgtcaagcccccttatatgctggtggtcggcaacattgtgatcaaacctgactcccagacaatcacttgcgaaaactgtcgactgctgacttgtatcgattctacctttaattggcagcacagaattctgctggtgcgcgcacgagagggagtgtggatcccagtctccatggacaggccctgggaagcttccccttctattcatatcctgactgaggtgctgaagggcgtcctgaacaggtctaaacgcttcatctttaccctgattgccgtgatcatgggcctgattgcagtcaccgctacagcagctgtcgcaggagtggcactgcactctagtgtccagagcgtgaactttgtcaatgactggcagaagaacagtaccaggctgtggaattcacagtcaagcattgaccagaaactggccaaccagatcaatgatctgcgccagacagtgatttggatgggcgatagactgatgagcctggagcataggttccagctgcagtgcgactggaacacaagcgatttttgtattactccccagatctacaatgaatccgagcaccattgggacatggtgcggagacacctgcaggggcgggaagacaacctgaccctggatatttccaagctgaaagaacagatcttcgaggcttctaaggcacatctgaatctggtgcctggaacagaggcaatcgcaggagtggcagatggactggctaacctgaatccagtgacctgggtcaagacaattggatccaccacaatcattaacctgattctgatcctggtgtgcctgttttgtctgctgctggtctgcagatgtacacagcagctgaggcgcgactctgatcaccgggagagagctatgatgactatggcagtgctgagtaagaggaaaggcgggaatgtgggcaagtcaaaacgcgaccagatcgtgaccgtcagcgtg

SEQ ID NO: 13 - DNA, 1980 bp, Equus caballus
  source 1..1980: mol_type = genomic DNA
atgcggcggctttcgctccgtgacgcaccctatcagcgtccacctcctcgatcaccgccgcctccacatccgcgctctagagccactcgagctgtgcctattcctacatggggtcaactaaaacaccttacccaagaggcagaacacttagttcaggtgcaggggggtacaatctccccctctactctgtttctagctatgttagccttgattgcctgtcaggcaggaaaaacctctgcctcccttaaccaatattgggcttatgttcctaaaccccctctattccatccagtaacctggaaggaaggacagattcctgtcacgaataaccatcctgagttattaggaggtctgtctatataccatgataaatttgacattaacaaagatttttcttttcaaggtacctctagcagtccccctatatgctttaatgtgcgccctgtagggcagatgccggctccactgccagtaactaacataccaggctgtattggaacctctattaaggggctacttacagactcccctaacggaatgtggagaaatttatggtccctgcaactattactgcctggggacatgggccccagggctctgaactctaccatccaccctccaggatatcctacttgtcctggaaggactacatccccagagagattgcctactaatagtgactggatgtcggttgacaaacgaacgggcttcccaaagtggagggagtgtatgtattcatcgaaaactgtgataaaaataccttcttttcccggtgagatatttgattggagctgtcaggacccttcgaaagattacagaaattatttacgtgaaactggtaaataccggtggtcagatcgttacatagctcctgaactggcggtggatcgttggcatatgaatggctgggttccccccatattgtcggcagactctggtgaggtccagcctcgcttatggcaggccgtagccgctctacacaacattatcctaaataggccagtggggtcccagtcctataaggttaaggcatgtattcctgccccttatgttttcctagctgcgcccactcacagtcgcgcaatgattattctacccaccacggacggttccatatataacattacctgtaccaattgtattctgactaattgtttaaatcctgatataaacgtggaaactgttatgatattacaacaacccccctatcttatgttacccgttcatttgtctggaccttggtatgatgatgttggattgcttactttgaaacaggctactgatcttttacagcgcccccgccggtttatcacggctttaatattgggtatctcagccttgattgcattagcaacctctctcactgtatctgctgtagcgctgactaaacaaatacatactgcctcttatgtagacgatctttctaagaataccactcttgccctaactacccaagaaataatagacgaaaaactggaggcaaaggtaaatgctctggaagaggcaatattacagataggacaagaacttacaaatttgaaggttagattggccttgcgttgtcatgtgtcctatagatggatctgtgtgactcctcttaaggtaaatcaatctctacactcttgggaaaaaattaaaaatcatatccagggaatatggaaccattcagactttagtctagacattagtgacttacatagacaaattaataatattaatcaggctgaagaatccttttctgcttctgaagtggccaccaactttttcacctctctgacctcttttgtaggggagaaaaattggagctcctttattgttaatattgctgtcaccattggtgctctaatgatttgtctttgcctttttccagtctttctcaagttaatattcaactctataagtcgggtttcagcagaattacatctgttggccttaaaaagtaaaaaagggggagatgctgggagccggagagtgaaataa

SEQ ID NO: 14 - DNA, 1761 bp, Mus musculus
  source 1..1761: mol_type = genomic DNA
atgactcccagatggatcccttggaaactacctctaatcctcatgctacctctgtggatttgggttcctagtatatttggagagtatagatgggcaattctgtctgctttccctaagcctatgcctgttcgtcataatacagctgtttttcccaagttttttacgaccaataaaactttaggcctgccatacctaccatttgaccctatctgggcaccattgggagaaaaacgctctttaagggaacgaggttctctctgtttccaaatttatgaattaggaggttgtatcagactcaccagccaagctttgggaatgttttttaagtatcgaggaggcgtggtgaaaataacccaagacacttccaacagagatataacccttacaaatcggaccttctggcatgaggcaacttgggttaatggtacatttctaccacctaacttttcagataaagaacgtcccaatcaaccaaaaatggctcctcattgtagcttggaagatgaagggctgatcctgccttggtctgattgtcaatcctctgtcactcgttgggcagatcagagtaaaactttttccttttctcccaacatgatagatgacccagagcagaaatatgttatgaaaaagggacttttcatacaggactttagaatgcatccttttcataaatgggtactttgtgggattaatggtagctgtacagatcttaatcccttggttttcctccaaggaggagctgctggaaaagcgatttttaatggtatttcaaaatttgctcagtttcatcaagttctgttacctgatagaaccatttatcaaaattctactactgaaatcactggatttaataagacattgataaagcagactaattatttacctactccagtttgtgtgtacacccctttcttgtttattctttctaatggttcctttgaatcctgcactaatgaaacctgctggatgtcacaatgctggagccttaagtgggccagccgtgcaatgcttgccaagattccacgatgggttcctgtccccgtggagaccccttccaccataaccttacacaggcaaaagcgagactttggtatcactgcagccgttgtggtggcaatggctgccagtgcggcggcagctacagctgctggaattgctatggcaacatcagtccagtcaagcacaactgtcgagcagttgtcctcctccgtggcggaggcgatcgatcaacattcagtgctcagtgcccaattaaaaggtgggctaatgatagtgaaccagcgcattgacctggtggaggaaagactggaaattctgctccaattggctcagcttggctgtgataagaaatcagtagccttatgtattactagtgtccaatatgaaaattggacacatgcagctaatctatctaaagaattgtctttgtttcttacaggaaactggtctgaaggatttgacgaaaaacttgaggcgctacgtacagcagtgatgacgattaattctacgcgcgtggacccatcattgatcgatggaatcaaaggactttcttcctggatgtcctctgcattctcacattttaaagagtgggtgggagtgggcctgtttggtgcaaccctgtgttgtggactagtgtttcttctatggctggtatgcaaactcagatctcaacagaaacgtgacaaggtggtaattgctcaagcacttgctgccatagagcaaggcacctcccctgaaatttggctatctattctcaagaat

SEQ ID NO: 15 - DNA, 1845 bp, synthetic construct
  source 1..1845: mol_type = other DNA; note = JSRV_env_ JSRV from pCMV2JS21_gift of Massimo Palmarini
atgccgaagcgccgcgctggattccggaaaggctggtacgcgcggcagaggaactccctgacgcatcaaatgcaacgcatgacgctgagcgagcccacgagtgagctgcccacccagaggcaaattgaagcgctaatgcgctacgcctggaatgaggcacatgtacaacctccggtgacacctactaacatcttgatcatgttattattattgttacagcgggtacaaaatggggcagctgcggctttttgggcgtacattcctgatccgccaatgattcaatccttaggatgggatagagaaatagtacccgtatatgttaatgatacgagccttttaggaggaaaatcagatattcacatttcccctcagcaagcaaatatctctttttatggccttaccactcaatatcccatgtgcttttcttatcaatcgcagcatcctcattgtatacaggtatcagctgacatatcatatcctcgagtgactatctcaggcattgatgaaaaaactgggaaaaaatcatacgggaacggatctggacccctcgacattccgttttgtgacaagcatttaagcattggcataggcatagacactccttggactttatgtcgagcccgggtcgcatcagtatataacatcaataatgccaatgccacctttttatgggattgggcacctggaggaacacctgattttcctgaatatcgaggacagcatccgcctattttctctgtaaataccgctccaatataccaaacggaactatggaaacttttggctgcttttggtcatggcaatagtttatatttacagcccaatatcagtggaagcaaatatggtgatgtaggagttacaggatttttatatcctcgagcttgcgtgccgtatccattcatgttgatacaaggccatatggaaataacactgtcattaaatatttatcatttgaattgttctaactgcatactgactaattgtattaggggagtagccaaaggagaacaggttataatagtaaaacagcctgcctttgtaatgctgcccgttgaaatagctgaagcctggtatgatgaaactgctttagaattattacaacgcattaatacggctctcagccgccctaagagaggcctgagcctgattattttgggtatagtatctttaatcaccctcatagctacagctgttacggcttccgtatctttagcacagtctattcaagctgcgcacacggtagactccttatcatataatgttactaaagtgatggggacccaagaagatattgataaaaaaatagaagataggctatcagctctatatgatgtagtcagagtcttaggagagcaagttcagagcattaattttcgcatgaaaatccaatgtcatgctaactataaatggatttgtgttacaaaaaagccatacaatacttctgattttccatgggacaaagtgaagaaacatttgcaaggaatttggttcaatactaatctatcgttagaccttttacaactgcataatgagattcttgatattgaaaattcgccgaaggctacactaaatatagccgatactgttgataatttcttgcaaaatttattctctaattttcctagtctccattcgctgtggaaaaccctgattggtgtaggaatacttgtgtttattataattgtcgtaatccttatatttccttgccttgttcgtggcatggttcgcgattttctaaagatgagagttgaaatgctgcatatgaaatatagaaatatgttacagcaccaacatcttatggagcttttaaaaaataaagagaggggagatgcgggggacgacccg

SEQ ID NO: 16 - DNA, 2064 bp, synthetic construct
  source 1..2064: mol_type = other DNA; note = MMTV_env q61_Gift of Susan R. Ross; genebank accession entry AF228552
atgccgaaacaccaatctgggtccccgatcggttcatccgaccttttactgagcggaaagaagcaacgcccacacctggcactgcggagaaaacgccgccgcgagatgagaaagatcaacagaaaagtccggaggatgaatctagcccccatcaaagagaagacggcttggcaacatctgcaggcgttaatcttcgaagcggaggaggttcttaaaacctcacaaactccccaaacctctttgactttatttcttgctttgttgtctgtcctcggccccccgcctgtgaccggggaaagttattgggcttacctacctaaaccacctattctccatcccgtgggatggggaaatacagaccccattagagttctgaccaatcaaaccatatatttgggtgggtcgcctgactttcacgggtttagaaacatgtctggcaatgtacattttgaggggaagtctgatacgctccccatttgcttttccttctccttttctacccccacaggctgctttcaagtagataagcaagtatttctttctgatacacccacggttgataataataaacctgggggaaagggtgataaaaggcgtatgtgggaactctggttgactactttggggaactcaggggccaatacaaaactggtccctataaagaagaagttgccccccaaatatcctcactgccagatcgcctttaagaaggacgccttctgggagggagacgagtctgctcctccacggtggttgccttgcgccttccctgaccagggggtgagtttttctccaaaaggggcccttgggttactttgggatttctcccttccctcgcctagtgtagatcagtcagatcagattaaaagcaaaaaggatctatttggaaattatactccccctgtcaataaagaggttcatcgatggtatgaagcaggatgggtagaacctacatggttctgggaaaattctcctaaggatcccaatgatagagattttactgctctagttccccatacagaattgtttcgcttagttgcagcctcaagatatcttattctcaaaaggccaggatttcaagaacatgacatgattcctacatctgcctgtgttacttaccctcatgccatattattaggattacctcagctaatagatatagagaaaagaggatctacttttcatatttcctgttcttcttgtagattgactaattgtttagattcttctgcctacgactatgcagcgatcatagtcaagaggccgccatacgtgctgctacctgtagatattggtgatgaaccatggtttgatgattctgccattcaaacctttaggtatgccacagatttaattcgagccaagcgattcgtcgctgccattattctgggcatatctgctttaattgctattatcacttcctttgctgtagctactactgctttagttaaggagatgcaaactgctacgtttgttaataatcttcataggaatgttacattagctttatctgaacaaagaataatagatttaaaattagaagctagacttaatgctttagaagaagtagttttagagttgggacaagatgtggcaaacttaaagaccagaatgtccaccaggtgtcatgcaaattatgattttatctgcgttacacctttaccatataatgcttctgagagctgggaaagaaccaaagctcatttattgggcatttggaatgacaatgagatttcatataacatacaagaattaaccaacctgattagtgatatgagcaaacaacatattgacacagtggacctcagtggcttggctcagtcctttgccaatggagtaaaggctttaaatccattagattggacacaatatttcatttttataggtgttggagccctgcttttagtcatagtgcttatgattttccccattgttttccagtgccttgcgaagagccttgaccaagtgcagtcagatcttaacgtgcttcttttaaaaaagaaaaaagggggaaatgccgcgcctgcagcagaaatggttgaactcccgagagtgtcctacacc

SEQ ID NO: 17 - DNA, 1659 bp, Heterocephalus glaber
  source 1..1659: mol_type = genomic DNA
atgaagacaatgcgcgatggttaccagaacggctcgtgcaccaggcagatgtctcttcagcagattctcctcctcctcatggcgatggtcacgatgactaagtgtgagatgttttgggcctatgtccctgacccccctcttttgcatcatcctgtcacctgggaggattcaccagtttcagtatatgtgaataatactgcaatagctggccccccctcactcggtcatataaaagaagagataaaagaaggttttaattattcagggaaaggatggggaatccctatatgtttttcacaaaatggtaccgtaaagagttgtttaaattttacatgggatacttttaatgaagggtctctgtatggcaacaagagacacaatattcgaatgccatggccaagttgtggaggctctctagagatagatgatcctccccaagactacccccaatgtatgaataaggcactcaccaataacagaatcataccatgggccagatgtagagtggaccggccggccatctataacttcaccgatcaacaagaagtgctacaggattggagtgcccaattggacaattgtcaaggtggggcgaacacagaacagcagttgactgtgggactttggcgagtgccttccggcccttttcagcccatgatatggaaacttatggttgctatgggagggataaaaaggattggtgtgggaccagactctgggttggagggaaatatcacagcttgtgtgccccagccctacttactgttattgggaggttttgagattgattatgaaaatggaactcgtcaatttaatgtaacatgtgataattgcacattgtcaaattgtgtgaaattcgggtatcaaaatgttttaataattcatcagccctcttttgtaatgttgccagttaatctttcagaaccatggtatgcagaaagaggacttcaaatattggagagtctaaatggtttattgaaaagagaaaaaagggtggccggtatgattgtagctggaattgttacctttatagcatttttgattagcttatctactgctgtggttgctttatcccagtctgtgcagacagcagcttttgtagataatctttcgatgaatgtatcacaagccttagagttgcaagaagatattgatataaaaatggaagagaagttgaatgctctttttgacacggtgatatatttgggacgctcccttgaggctctcaaacatagaaaccatttacagtgccatggagaatatcgttggatctgtgtcacatcactagcttataatatgacagattataactggcagatggtccaggaccatctaaaaggggcatggtcacatggcaatgaaacctcagacctattaaagctccatcagcagatattgagcattcaacaagcgggaagaataagtctgtcacctgcacaggtggcacagtctctttatgatcatttaaaaacattggtgccttcctcattctttgctaaacagatagtgagaggccttggactactggctgtcggacttctgcttatgtttcttttttttgtcatattcagccagtatgttcgatccatgttcttaacactggctgttcaattacattatcaaaaattgatcacctctaagaggccggttagtcaatga

SEQ ID NO: 18 - DNA, 1998 bp, Monodelphis domestica
  source 1..1998: mol_type = genomic DNA
atggagagatctggactcccagtaagcatctcaaaccgtggaagggaccgcttccagatgcaaggggagcagaggaacaggctcaccagtctagcaccgcaccggctgcccgagaccaggacgagaagcagacgagcatcgctgctgtctccagtcagctacctgatggtcaagcgcagggcaccgggatcgaaggcaacactggtgaatgcacgcactctgaacactgacattacatttactctgatggcattcactattttgactttgctgaacatctgtcatgctgaagtctactggtccatcgtgcccaaacctcctattttgagaatgttaacttggatggacaagccccctctggtatatgttaacaacacggactggctaccacaccctattctagccaaacgtgttcctctagagtctgaaggggctctgtacaattattcaggatctactgtgtatcctccattttgtatgtcttttagaaattcgtttattggtaattctttctgtgtaccacgcagacatcaagcgtggattgtcaaaaacgccactgacaacagttatgtaggggttggcatgggtgtgataggaatgggagatgagttagcacgtaagacgttccaacctaaacaccctgctaacaagtacctatgtccaaatcagataggtacggtacaaaaggagttgccatggacagattgttctgtccgagttcccagaaaggttaagatgccggcaaccacagactttaacatttacaattgggcacccaggttacgttgtggtcgctctgaacaactgacacggttagacaaatacactgactatgagaaatggcacatgccatgtcagaattttcagaaaattgaggacttactagaagtagacatggctggttctagacttgctattgaagcaggtcctatacagaacacacagtggaagatagtggctgccacgcagccgatatacaagtttaaggtcaaggtgaagaactcaggacttgatcttgagtatgacagtcacatcaatgtcacagcatgtgtttttgctccttatgtaatgctagtaggtaacaaccttcgaatttttaaaaatgacaaaggtctatatgagatcaactgcacaaaatgccgactacatcaatgtcttagtcctaaacaccaagattcatatgttgctatcatgtatcatccacctttaatgtgggtacctgtcaacttgacagagacgtgggcatctacgcctactgtgcacattgttcaaaaggcatttaacatggttatacataggtccaagagaatggtctttgcacttgtaggagccgtagtctcagtaattgccatgatcacatcactaaccacggcaacattggcattaacttcctctatccagaaccatgagttcatacaggaagtggtgtctaactcttccaaattatggcacactcagcagagtattgacttagcttacagagatgagctggacagtttaaaagatgctgtgttttggttaggtaaagaggttgaagctttacacacaaggattacttatccctgtcattacaatcaaacgggttattgtattactccgttgccatatgataatgtgtcatatgaatggcaactggtcgttcagcacatcaagggtgcttggggaagtcataacagcaccttggacattattaagttgcaacagcagatcaacaaattagaccaaattgtatatgagaatgaagctgcaaatatagctgcaaattgggaagcaactttatcttccctagatccccaaaattggtttggtagagctaatttaaccagcattggtactatcattttattcatattattggctattggtttgattatactttgcagcagaagctgtaaaaacagagcctacattcgaggcatactgccagaattggcacggtcttatcacactaggcaattggtgcctgaaaatgttgaattgaacacaaccgttttatga

SEQ ID NO: 19 - DNA, 2070 bp, Rattus norvegicus
  source 1..2070: mol_type = genomic DNA
  variation 1081: note = n is selected from A, T, C, G
atgccgaatcaccaatttgggtcccagacagactcatcaggccagccaaaggaaacggacatccgtcccccacgccaaaagaggccgaaccgccggataatgcggaacctggccaaaaagatcaaggcgactcgcctagagccaaaaataaaaccacgccagacgccgacgtggacgcaacttaaaaccctgacagctcaggcaaaggaagtattgaaggctgcacatgccccggaaacggcaaccttgctgtttgtggctatgcttgcagtcatgagtgcgcccaccgctttaggggaaagttgttgggcttacttaccaaagccacccatattacatcctgtagggtgggaggtgtcggatcacatcaaggttttaaccaatcaaacattagtgatagggggatcgcctgactttcaccttctaaagaacagctcgggttatgtagattttgagggaaagtctgattctctgcccatttgcttctctttctccccttcagtgccagtgggctgttttcaagtggggaaaagggtgtttaacacagatacccccaccttggataataccaaactagatggaaagggcaatgggcggcgtatgtgggaattgtggatgaccaccgtgagcgataaagaagaaaaaagacaacattataccacagcaaagaatcttccgccgcgctacccccattacagaacaacatttaaaaaggacgccctttgggagggagatgagaatgctttcccgcgctggcttccatgtgccttccctgacaatggggtaagattctctcccttaggcgctgctggacttctctgggacttctccatgtcctcacctgataaagatcagaacaattatctctccaccaactcaaaactttatgggaactacgtgcctccagttggaaaaccgatcaaaagatggtatgatgcggggtgggtagaaccaacatggttctgggagccactccacacggacctccctgatagacagaacaaaccactgatagaacatccagaactgttccgactggttgcttctagtgagaaacttttgctgaagaaaccaggagctngcgattatgatgctgtttcagtttctgcttgtgttacctatccctatacaatattagtaggattgccacaattgattaacatagataaggttgaatctatttttagtattaagtgtacatcatacaagctgactaattacattgattccactgtatcgggatctgttatattgattgtcaggagacctccttatgttttgttaccagtagacttgggagatgagccatggtttgatgattctgccatccagactattaggtatgctacgggtttgattagggcaaaaagatttgtagcagccttaattctaggtataaccgctttgatagctattgttacttcttttgctgtttctactactgctttggttaaggaaatgcagactgccacttttgttaatgatctccataagaatgttacccttacattatcagaacagaagattatagatttgaaattagaaactagattaaatgccctagaagaggtagtattagaattaggacgagatgttacaaacattaagactaggatggccacacgctgtcacgctaattatgattttatttgtgtgactcctttaccttataatgctacagaagaatgggaaaggaccaaaacacatttgctaggcatatgggatgatgataacatttcttataatattcaggaattgacgtccttaatcacagaaatgagtaagcaacatgtagatgcagtggacctctctggattgacaagatcctttgcggatggagttaaatcattaaatccaatcgattggacacaatattttatttttattggagttgggattttactacttgttgttgtactaatgatcttcccaattgtattccgatgctttacagattccattgcccaggttcaaacagatctcaatttggtgcttttaaaaaataaaaaaggaggaactgcggtgcctacagcagagataaccgagctccgggacaaacatgacgcctga

SEQ ID NO: 20 - DNA, 2073 bp, Macaca mulatta
  source 1..2073: mol_type = genomic DNA
atgaacccatcggagatgcaaagaaaagcgcctccacggagacagaaacaccgcaatcgagcaccattgactcgcatgatgaaccaagtgatgatatcagaagaacagatgaagtcaccacgcaccaagaaggcggagctgccgacctgggcacagttaaagaagctgacaccgttagctggaaaaagcctagctagcacaaaggtgacacaaaccccagaaaaaatgctgcttacagctttaatgattgtatcaacggtggtaagtctccccatgcctgcaggagcagctgcagctaattatacctactgggcctatgtgcctttcccgcccttaattcgggcagttacatggatggataatcctattgaagtatatgttaataatagtgtgtgggtacctggtcccacagatgatcgttgccctgccaaaccggaggaagaaggaatgatgataaatatttccattgggtatcgttatcctcctatttgcctagggagagcaccaggatgtttaatgcctgctattcaaaattggttggtagaagtacctactgtcagtcccaccagtagatttacttatcacatggtaagcggaatgtcactcaaaccacaggtaaactatttacaagacttttcttatcaaagatcattaaaatttaggccaaaagggaaaccttgccccaaagagatttccagagaatcaaaagatttagtttgggaagaatgtgtggccgatagtgcagtgatattacaaaacaatacattcggaacagttatagattgggcacctagaggtcaattctaccacaattgcacaggacaaactcaattctgtcccagtgcactagtgagtccaactgttgacagtgatttaacggaaaatttagataaacataagcacaaaaaattacagtctttctacccttggatatggggagaaaagggaatatctactccaagaccaaaaatgataagtcctgtttttggtcctgaacatccagaattatggagacttactgtggcttcataccgccttagaatttggtctggaaatcaaactatagaaacaagagattataagccattttactctatcaacctaaattccagtctaacagttcctttacaaagttgtgtaaagcccccttatatgttagtcataggaaatatagttattaaaccagactcccaaactataacttgtgaaaattgcagattgtttacttgcattgattcaacttttgattggcagcaccgtattctgctggtgagagcaagagaaggcgtgtggatccctgtgtccatggaccgaccatgggaggcctcaccatccatccatattttgactgaagtattaaaaggcgttttaagtagatccaaaagattcatttttactttaattgcagtgattatgggattaattgcagtcacagctacagccactgtggcaggagttgcattgcactcttctgttcagacagtaagctttgttgacaattggcaaaagaattccacaaggttgtggaattcacaatctggtatcgatcaaaaattggcaaatcaaattaatgatcttagacaaaccgtcatttggatgggagatagactcatgagcttggaacatcgtttccagttacagtgtgactggaatacgtcagatttttgtattataccccaagtttataatgagtctaagcatcactgggacatggttagacgccatctacagggaagagaagataatctcactttagacatttctaaattaaaagaacaaatttttgaagcctctcaaagccacttaaatattgtgcctggagctgaggcattagatcaagtggcaaaaaatctttatgaattaaaccccacgacttggattaagtctattggaaactctactgcaataaattttggaattatgtgtctctgtttaatcggcttgtttttagtgtgctggaccagtcgaagaatcctgcgtcaaaatcgagagaacgaacaagccttcatcgccatggcacatttatatagaggaaaagggagggagaacgttgcgggaagtcagggaccttga

SEQ ID NO: 21 - DNA, 2334 bp, Condylura cristata
  source 1..2334: mol_type = genomic DNA
gatcatcttcaaaccaagcagcgcttcaataaactgtgatagaagaagaatcctgagttcgtgtttcattccgcgctggacgcgggtcgcaacagctggtgccgaaacccgggaacgcatcagtaggaactccagtggggggccgtattaagaagattcagaacttcacacaggaagcggtcgacgtcggtaagtatcttcaggatcgaccggacccgatttggctccctacacggctgacgagagaagtggcgagaatcccagacggggacgatgctgaaggtcgcgagaacattcctgctcctgcggatgctgataatagtcccctcagctagcctcgataactatttctgggccatcactcgtacgtggcccactccttttcctgtgcataacgagtccctggttttgcctcaattttatgctacttcttgcacctatggcaccccttgcaaccctaagccgaccagtatccaagaagaaaaatttaatgcttctagattcagcctggccgggaccttgtgcttctcggttgctctcaatgccactgactgcatcctgctttccgccaccaatctctctgtattcgctgacccactcttgcatctagaccctggactttccgcttcagtgcttgtttcagccctgacacaggcagccgcgggcaaaactcaaggatccggttctgcaaatgggacatcggatgctgtcaatgttaccacctttttggtcaacgtctctgcgatcactcccaacctcatttccaccagcgaaaactggaccttcaacaggtcctaccctttaacagcttgcagaaagacggtgttgacctttcctcccgagtttgctgattgcagggatacacgcaggcccgcccaaaaggatggttgggccctctggcccagccgattcggccgctggaaggaaaaacgattgatcgccgaccatccgctgtcggcttgggttcttcaaaatgtcaagggtgcgacttgtgacctctcccccttcattcatctgcagccctcgactatccatttgtctaatgttacggggtgtcttaacacgacttcaaaaaaactcaccttgcacacacatcggttgaaggataatttcacttctcctggaggggttgtttgtgtcgaccccccttttctttttctgctttttaatgcgtcttcagaatcttctattgactgcacaacgacaaattgcttgttatcccagtgttggcgaaagacacatacatatgctcttagtctgcgcctaccaacatttgttcctttgccagtgcaagctaacccacaggatttccctgtcaccgccttgatccgatcaaggtgcgattttggacttaccgcggctctggtggcggccatagctgcgtccattggtgcggctgccgccgctgcggttgcgatgcagacctctgttcaaacggtagccgtggttaacgaggtcatccaaaagacttcctcttcgctacaaactcaagagcgccttaatcgtcatctcacctctggcatcttacttttaaataagcgcattgatttaaaatcaatggaaagtaatgttctgactttatttgatattgttagccttagctgtgtcgctaagaccccacatatgtgtgtaactccctattatgcttcattgaatgagtcgcgccgcttgagttccctcctcgctggtaattggacgagggaatttgaatgcttgcaaaataactttactgcacaaattgttgctcttaacatgaccaaggctgaagtggtgactgtgcgccagtttacggattggctttggtcctctttttcctttttcaaggagtgggttggtgttggcatgtttgctgcagttcttgtggcaggcctactgctactagtatttctttggtttcgaaccgtgcgtgcgcaccggcgggacaaggcggccatcactcgagcattgatcgcaatggatgctggccactctccccaggcttggattagcctcctgcgtgaagcctgactccttgcccttgcacccgaggggcgttccggcccattgcacttggagggatggagccagtgcatggccggtagcttgcgggtcgcaacaattctggttatttataatgaggtttttttttctaactagggatgataactgttttggtttggttgcttgtgctttagggaagggggcattggcctttattgcttttgttttctttgcttgttttaaaacttgaaaggattatgggaatgtacatttggtctttgtttatgttcctactgtttgtccaagccacttgtctttagctttggttatggagcaactgcaggtttaaatata

SEQ ID NO: 22 - protein, 584 aa, Loxodonta africana
MQDPKTTKIPPTLPPSLLTNLTPPTTLLRNLSLENAPSTPPPSPVSSSPSSSSSTPPLLTDFIQPSTNLNNSSSRDKVRWRSRRHTPYSRTHQTIAAEPPTWGQLKKLTHLAHILTVDQHIPVNAGTLFVAMLAIISCQVCVVMADMHWAYVPKPPIFHPVTWEDPSPSVFTNNSALLGGETIFWNPQGFNSNYSYSGMSDNPPICIQLRNLRKTIPGCIPFGFKSMITDSPNGNQNQQPYRLLWALRLILPEKTDFLKGIPKVHTPLFPTCDKSPPEDSKSNQDWAMIDPSKGFPIWRNCMYNSQVVYALPKMSARLIDCSILNPEDDYRLFLQSARYRFNWQDTLVPLPQKVNRWHINGWVPPILSTFTGNIHPSLWRLATAAGNVTLSRPFNNESFDIQACVPAPYVLLISRNNHSSIVIENKKKHYVSSCKNCILTSCINPAIPVESMMILQQPKYIMIPVHLQEDWYDDSGLEALKQASAILSRAKRFVAALILGISALIALATSLSLSAMALTQQIHTANYVNNLSKNITLALATQEAIDRKLQQEVNALEEAILYIGQEITNLKVRLTLRCHSSSES

SEQ ID NO: 23 - protein, 672 aa, Mesocricetus auratus
MLVCSHRMKAHRSGFQTDLSDPTSRRLSRKTKRWRNRELQTMGKQLTSLTITNKEGEVTSEGELSCNLPTQTQLKNLQGEALTVRAQTKTPKTPVAIFLAFLAILSMNSAVNGESYWAYIPKPPLLHPVVWGNTELIKIMTNVTDVLGGTPDFHMGINSSGYINYEGRADSLPICIALDGMPPAGCLPVSYRTFLTDTPDNKPVTSRSPEQRHMWQLQIATLGHSNFNETMVVNQPLPSRLPHCMVKYKKDSLWEGDEDQMPNWLHCGFPQAGTRFSPLTSGEIIWDFSVPSPDHDYSGYLKDHSAQFGGYTPPKNEPIRRWYSAGWVSPVWYLQKPDGTYGTPQPDLFRLVAAANVIILKKPKAAKSEPVPATACLSYPYALMFGRNKLVHIKKKGTHFFVKCRSCILTNCVQSGYDKSFSTVLIVKRPAYVMLPIDLGNDTCYDNLAINLFNKINELIRPKGFVASLILGITALIDILTSFTVSTTALVKEMQTASFVDDMHRNVTFALSEQRLIDLRLETRLNALEEAIIEMGQDIQNLKTQLATKCHAEFKYICVTPLPYNSSFNWNKTKAHLLGVWKDTNLTYDIKALQEKITVMSQKKIDTWDISGLASEFSENLRSLNPLSWTQYFVFLGIGAAILFTVCLVFLFFSEYHFTPSAPSSVISSCSS

SEQ ID NO: 24 - protein, 687 aa, synthetic construct
  source 1..687: note = BoRV_CH15_Env Bos taurus Bovine retrovirus beta-like env NCBI accession AMS37619
MVLFFCRMENNNGSRRVGFKYGGPPAKKQLLQPPRFDPTVTVPEIEIPYQAFCRMTLGERRRKRPVTVPLPTWGQIKKLSQEAFRTVMGTGKVPTPENLFLAMCAILTVTSSQAASLPVPTPEADVSYTYWAYIPNPPLLEATVWGVSDILIYTTPHVLSPPWKNLTYLNKDDGRLFNFSQRLTQGTPICLTKEARDPCLVMDWQSWLLPGRNESSIRARLQRLTTWSINNSYNNISLQETASVPFCTPFTYISLKYKPFIWSECTSEWAKRTGDYINWGPFGMFFNDCSEWNNKTCLFFNTTRLLMPMNQTLQGYKLMWWGDGGVSNPRWTHVQYPDNYQSHLWKVAAALQPVYKAQGQFLGTSRSFSTYSFFNITQYNLTACIPLPYLFLVGNFMYNEGFIQCNNCALYSCLNASIDAPAIMILQQRTHLWLPVKMEQPWASSPLDKIIIQVLKKVLHRSKRFIGAVIATVLSLIAIVTVATVSSVALSTAIQNKHFLETWHQDSFQFWTEQTQIDARFQQQIDILKQSVQWLGRKMIQLEKQISLKCHWNSSTFCITPILYNKTESDWVVIQHALEGNMTAVQLIDDLQKEVNQEFGKPYSNDYSGEIADDIFKHLEKLNPFTWGQSLIHSAGSLGVSILILIISLILIYRCCIVPLQLSLTQMWTKMLISKKKKGGIEDIGKS

SEQ ID NO: 25 - protein, 584 aa, Bos taurus
MFVFSHRMPKRHAGCWKGWYARRRRSLANQMRRLTIQEHDELPSRQQLQTLMREAQNSVAVQGDPISPLSFLITLLFILNQINTAHSEEYSSAYWAYFPNPPLLQPVGWEGRSIPIYTNDTVALGGFSDKHIIPNQVNFSYHGVFDRLPICLSRYFNSTGCLFVGESGYADYDNGCSLYIPGVMKESGIPQRRNGTGPLDIPFCDFGTRGRVTAAPWKLCRDGIATVYNIFGTNESLWDWSPDSARNPGAQDNRKFASRIWNLGGSKVFQTEIWKLAAALGCEGSYLQVGSKVRHGYLWNLTMRYPRACVPHPYALLVGSVNIQPGLRNYNVTCNNCTLTNCIRGITNQTRVLVLRQPAFVMVPVKINGSWYDERGLELWREVEGALMLYRRGIGLIILGFVALVTLTASWITAALSLAQSVQTATFVNNLAQNASVALGTQEDIDKKLEDRLNGLYDVVKFLGEEVQSIKLRLRVQCHADFQWICVTPKKYNGTITAWDKVKAHLEGIWHNENISLDLLHLHQEIMNIENAPRLDLDVAKRAESFVKELFRHVPTVNSIWYLGAAIGGLFLIILTFISCTLYN

SEQ ID NO: 26 - protein, 603 aa, Bos taurus
MTPQAEQTLKVTGTPMTPDKLFLAMLAVLSCSSGVSVKYLYWAYLPNPPLLSILDWVNKAPVVFTNDSLHFPAPWSTQRPIHEEDEGRKINISIGFKTLPICFGFHPLCITIFPQWWAYSAHNGSAYRIGMFATTSFLLNGSERHYPGQTVNYINSLFQPEEPICDVVSWRRKQSIQSLYWSDCQGQFGKISIIQQNHFIFKFIDWGPHRLFVNCSEEQEKNHNCSWYNKSNVWGFHKTNTSFWTDKDILLNWYNGGLSPPRPRIIVPTVGPEQWRVWKIPAALEHFASVYGTVKALKPQNYTFLYNSTGYIQSCVHLPYMFIVGNFDMDLSAKIVNSTACALYTCLNHTISYHNVNIAVVKQRSELWLPVNLTEPWTDSVFLSVLLKYGLRRSKCIIGWIIAGILSLISIVTVGTLSGMALHNSIQNHDFITAWHKDSHNLWTQQAQIDQQLQTRINELQTVVINIGDQVQQLTFLTHICCHWNFTSFCLTNMPYNGTEYPWDKVKVHFQDLMDNSSLDINLLKQQIANFQVKIPKELYSTQFYDILTKTLSSLDPRTWFSGGNFFMYVLIALLFVIVCRIQMSLLKTLGLPQWSPTSSCHA

SEQ ID NO: 27 - protein, 713 aa, Bos taurus
MSQPPRHLVPPLPRPQKRKGPPLPPLPPPTREAQLISRRMTPEILDEQEEEPPTRQMSQLCIRDIVLPNSLPHRKSPGCPTRATQQASIPTWGQIKTLCHQAQGIASLQGSPVSPERVFIAMLALLSCQVSASSPAPEKYWAYFPDPPTFQVVTWNNAPIRVHTNQPHLLGGHYTFYTEEEYPINFNYTFRGLTDDLPVCFNFPFDHTGSYITPTKEGCIGASEKAIITDSPSLRYRSVWVLLARMPGILDPYKSLHFKSPPKYPNCHNAAPSDAIWKTIDGHSGYPIWKSCTYNSRIDYRIPGGNYTIQDWSNPDPGQDPMIDDAFKGRFDNWKEYSVPWPLRATRWHRNQFVPPMLSYTAKGRTYWQPEIWKALTATAPITLTRPENISTYSILACLPSPYVFLFTNDSKRLNIHMNYSGGPNIVTCEQCMLSSCLAPQYNVCSFVVLQRPPYLMVPVTITTHWYDNYGLAVIQQVRDLMRSRRLAGLLLLGIATLITGITSVYMAAVSLTEQVHTAQYVDAMSKNVSLALATEEAIFRKLEMRIDALEEAILHIGNELQALKVRLALSCHADYRWICVTPLKVNETDYDWQKIKNHILGVWNSSDISLDLRKLHSQITTIERSHLDFTASGVASDFFHTISNFVSGKYFLSAIFNYAAVAAIILLIIFILPCIVRIFRQSIQKLETELHTGFLRNKNGGDAGTQHGSSHP

SEQ ID NO: 28 - protein, 704 aa, Bubalus bubalis
MSQPLGYRIPQLPRPRKRKGPPLPSFPPPARETPPASGQLTSEIPDEQGTDPPTKQMSQLHIQDIAPPDPLPHKRVSGRLTQATRNASIPTWGQIKTLCHQARGIASLQGSPASPEKLFIAMLALLSCQVSASPIPTKYWAYLPDPPTFQVVTWDNEPIRVNTDQPQLLGGSYTSYTRDKYPINFNYTFRGLTGDFPVCFNFPNDLRSFITPTKEGCVGASKKIIITDSWSLRHRSAWVLLARMPGILDPYVTLHSKSPPKYPNCYKVAPSEKVWNTIDSRTGYPTWTSCTYDSRIDYRIPGGGDYTIQDWSNPNPGEDPLANDTFEGRFENWDRIPLPWPTHATRRHRNQFVPPMLSYTTKKQTYWQPDIWRALAATAPVSLYRPDSNSTYSVLACLPSPYVFLFTNDSKKLNVRMNYSGGPNIVTCEQCMLSSCLTPQYNVCSFVVLQRPPYLMIPVTVTSHWYDNYGLAVLQQLRDLMRQRRFVGLLILGISALIAAITSVTAAAISLSQQVHTAQYVDTMSKNVSLTLATQEVIDRKLEMRVDALEEAIMHIGTELQALKVKMALSCHADYRWICVTSLKVNETDYEWEKIKNHISGVWNSSDIGLDLGKLHNQIQTLEHSRLDFTAAGAANDFFHTFSDFISGKNILSNILGYIAAGALVLLLIFILPCIVRILRQNIQKLATELHLAVLRNKKGGDAG

SEQ ID NO: 29 - protein, 519 aa, Canis lupus
MRTPRAGSRSDGVDSSPLRRRLANLTITRQQKRRRRRLPSYCWARIRELINQAISLAEQTQPNESLLTILLTVLAITTPPVGEAAVYWAYLPDPPTIRPVTWDDPTPQIHTNMTEYFGGSSSDDMEVANHSINWTGLAAQVPMCFQLPLCDQKPVNGCLVVQPVPHIVTYLSEMEKNENPPSRLNIMLERWIVYEVTGDTPPSDPQQGRRPPGYPNCPQNLTYAISGPIWTECLQKTPLVISPHGIPEFSITDWSRMTRKEPLRYIAYPPGRPMHNVTIPGGVFSTVGEMYHAELWKLVTAMGPAKRQMAKSGYTNTKDFNLTIQACVPKPFLLVVGYGTTNFSFMQNNGTDMLFELGCKNCVLTNCLPVHLPSPQTGPITIFLTMQPPYLLLPVEIEGPWYANYGYQFAVELQLQLKRLKRFLGLLIAGIAALVALIATAAAASVALSQGIQTAQYVNNLAKNVSYALSTQERIDQKILSCLDGLEEEKSGVKGILESILSSSSMSQQDNKLRDSHGL

SEQ ID NO: 30 - protein, 692 aa, Equus asinus
MYSRRCRTLPPLRRTDNSALTETDEDHPPVIQMRWLSLRDAPYQRLPPRSPPPPYPRSKATRAASIPTWGQLKHLTQEAEHLVRVQGGTISPSTLFLAMLALIACQAGKTSASLTQYWAYVPKPPLFHPVTWKEGQIPVTNDHPELLGGLSIYHDKIDINKDFSFQGTSSSPPICFNVHPVVRMPAPLPATNIAGCIGTSIKGLLTDSPNGMWRDLWSLQLLLPGDMGPRVLNSAIHPPGYPTCPGRTISPEKLPTNSDWTSVGKRAGFPKWRECMYSSKIVIKIPSFPGEIFDWSCQDPSKDYRNYLRETGKYRWSDRYIAPELAVDHWHMNGWVPPILSADSGEVQPHLWQAVAALHNIILNRPVGSQSYKVKACIPAPYVFLAAPTHSRAMTILPTTDGSIYNITCTNCILTNCLNPDIDVETVMILQQPPYLMLPVHLSEPWYDDVGLLTLKQATDLLQRPRRFITALILGISALITLATSLTVSAVALSKQIHTASYVDDLSKNTTLALTTQEIIDEKLEAKVNALEEAILQIGQELTNLKVRLALRCHVSYRWICVTPLKVNQSLHSWEKIKNHIQGIWNHSDFSLDISNLHRQINNINQAEESFSASKVATNFFTSLTSFVGEKNWSSLIVNIAVTIGALMICLCLLPIFLKLIFNSISRVSAELHLLALKSKKGGDAGSHGYII

SEQ ID NO: 31 - protein, 699 aa, synthetic construct
  source 1..699: note = HERV-K108_Env NCBI Accession: AAD51798
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPTEVYVNDSVWVPGPIDDRCPAKPEEEGMMINISIGYHYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 32 - protein, 699 aa, synthetic construct
  source 1..699: note = HERV-K113_Env HERV-K113 Env NCBI AAL18258
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEIYVNDSVWVPGPTDDCCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTLIDWAPRGQFYHNCSGQTQSCPSAQVSPAVDSDLTESLDKHKHKKLQSFYPWEWGEKGISTARPKIISPVSGPEHPELWRLTVASHHIRIWSGNQTLETRDRKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQNNSTRLWNSQSSIDQKLANQINDLRQTVIWMGDRLMSLEHRFQLQCDWNTSDFCITPQIYNESEHHWDMVRCHLQGREDNLTLDISKLKEQIFEASKAHLNLVPGTEAIAGVADGLANLNTVTWVKTIGSTTIINLILILVCLFCLLLVYRCTQQLRRDSDHRERAMMTMVVLSKRKGGNVGKSKRDQIVTVSV

SEQ ID NO: 33 - protein, 699 aa, synthetic construct
  source 1..699: note = HERV-K_Phoenix_Env HERV-K Phoenix Env extracted from pCRV1-HERV-K Phoenix env
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIISPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSIHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 34 - protein, 659 aa, Equus caballus
MRRLSLRDAPYQRPPPRSPPPPHPRSRATRAVPIPTWGQLKHLTQEAEHLVQVQGGTISPSTLFLAMLALIACQAGKTSASLNQYWAYVPKPPLFHPVTWKEGQIPVTNNHPELLGGLSIYHDKFDINKDFSFQGTSSSPPICFNVRPVGQMPAPLPVTNIPGCIGTSIKGLLTDSPNGMWRNLWSLQLLLPGDMGPRALNSTIHPPGYPTCPGRTTSPERLPTNSDWMSVDKRTGFPKWRECMYSSKTVIKIPSFPGEIFDWSCQDPSKDYRNYLRETGKYRWSDRYIAPELAVDRWHMNGWVPPILSADSGEVQPRLWQAVAALHNIILNRPVGSQSYKVKACIPAPYVFLAAPTHSRAMIILPTTDGSIYNITCTNCILTNCLNPDINVETVMILQQPPYLMLPVHLSGPWYDDVGLLTLKQATDLLQRPRRFITALILGISALIALATSLTVSAVALTKQIHTASYVDDLSKNTTLALTTQEIIDEKLEAKVNALEEAILQIGQELTNLKVRLALRCHVSYRWICVTPLKVNQSLHSWEKIKNHIQGIWNHSDFSLDISDLHRQINNINQAEESFSASEVATNFFTSLTSFVGEKNWSSFIVNIAVTIGALMICLCLFPVFLKLIFNSISRVSAELHLLALKSKKGGDAGSRRVK

SEQ ID NO: 35 - protein, 587 aa, Mus musculus
MTPRWIPWKLPLILMLPLWIWVPSIFGEYRWAILSAFPKPMPVRHNTAVFPKFFTTNKTLGLPYLPFDPIWAPLGEKRSLRERGSLCFQIYELGGCIRLTSQALGMFFKYRGGVVKITQDTSNRDITLTNRTFWHEATWVNGTFLPPNFSDKERPNQPKMAPHCSLEDEGLILPWSDCQSSVTRWADQSKTFSFSPNMIDDPEQKYVMKKGLFIQDFRMHPFHKWVLCGINGSCTDLNPLVFLQGGAAGKAIFNGISKFAQFHQVLLPDRTIYQNSTTEITGFNKTLIKQTNYLPTPVCVYTPFLFILSNGSFESCTNETCWMSQCWSLKWASRAMLAKIPRWVPVPVETPSTITLHRQKRDFGITAAVVVAMAASAAAATAAGIAMATSVQSSTTVEQLSSSVAEAIDQHSVLSAQLKGGLMIVNQRIDLVEERLEILLQLAQLGCDKKSVALCITSVQYENWTHAANLSKELSLFLTGNWSEGFDEKLEALRTAVMTINSTRVDPSLIDGIKGLSSWMSSAFSHFKEWVGVGLFGATLCCGLVFLLWLVCKLRSQQKRDKVVIAQALAAIEQGTSPEIWLSILKN

SEQ ID NO: 36 - protein, 615 aa, synthetic construct
  source 1..615: note = JSRV_Env JSRV Env extracted from full length pCMV2JS21 gift of Massimo Palmarini
MPKRRAGFRKGWYARQRNSLTHQMQRMTLSEPTSELPTQRQIEALMRYAWNEAHVQPPVTPTNILIMLLLLLQRVQNGAAAAFWAYIPDPPMIQSLGWDREIVPVYVNDTSLLGGKSDIHISPQQANISFYGLTTQYPMCFSYQSQHPHCIQVSADISYPRVTISGIDEKTGKKSYGNGSGPLDIPFCDKHLSIGIGIDTPWTLCRARVASVYNINNANATFLWDWAPGGTPDFPEYRGQHPPIFSVNTAPIYQTELWKLLAAFGHGNSLYLQPNISGSKYGDVGVTGFLYPRACVPYPFMLIQGHMEITLSLNIYHLNCSNCILTNCIRGVAKGEQVIIVKQPAFVMLPVEIAEAWYDETALELLQRINTALSRPKRGLSLIILGIVSLITLIATAVTASVSLAQSIQAAHTVDSLSYNVTKVMGTQEDIDKKIEDRLSALYDVVRVLGEQVQSINFRMKIQCHANYKWICVTKKPYNTSDFPWDKVKKHLQGIWFNTNLSLDLLQLHNEILDIENSPKATLNIADTVDNFLQNLFSNFPSLHSLWKTLIGVGILVFIIIVVILIFPCLVRGMVRDFLKMRVEMLHMKYRNMLQHQHLMELLKNKERGDAGDDP

SEQ ID NO: 37 - protein, 688 aa, synthetic construct
  source 1..688: note = MMTV_Env MMTV Env from q61 plasmind - Gift of Susan R. Ross; genebank accession entry AF228552
MPKHQSGSPIGSSDLLLSGKKQRPHLALRRKRRREMRKINRKVRRMNLAPIKEKTAWQHLQALIFEAEEVLKTSQTPQTSLTLFLALLSVLGPPPVTGESYWAYLPKPPILHPVGWGNTDPIRVLTNQTIYLGGSPDFHGFRNMSGNVHFEGKSDTLPICFSFSFSTPTGCFQVDKQVFLSDTPTVDNNKPGGKGDKRRMWELWLTTLGNSGANTKLVPIKKKLPPKYPHCQIAFKKDAFWEGDESAPPRWLPCAFPDQGVSFSPKGALGLLWDFSLPSPSVDQSDQIKSKKDLFGNYTPPVNKEVHRWYEAGWVEPTWFWENSPKDPNDRDFTALVPHTELFRLVAASRYLILKRPGFQEHDMIPTSACVTYPHAILLGLPQLIDIEKRGSTFHISCSSCRLTNCLDSSAYDYAAIIVKRPPYVLLPVDIGDEPWFDDSAIQTFRYATDLIRAKRFVAAIILGISALIAIITSFAVATTALVKEMQTATFVNNLHRNVTLALSEQRIIDLKLEARLNALEEVVLELGQDVANLKTRMSTRCHANYDFICVTPLPYNASESWERTKAHLLGIWNDNEISYNIQELTNLISDMSKQHIDTVDLSGLAQSFANGVKALNPLDWTQYFIFIGVGALLLVIVLMIFPIVFQCLAKSLDQVQSDLNVLLLKKKKGGNAAPAAEMVELPRVSYT

SEQ ID NO: 38 - protein, 552 aa, Heterocephalus glaber
MKTMRDGYQNGSCTRQMSLQQILLLLMAMVTMTKCEMFWAYVPDPPLLHHPVTWEDSPVSVYVNNTAIAGPPSLGHIKEEIKEGFNYSGKGWGIPICFSQNGTVKSCLNFTWDTFNEGSLYGNKRHNIRMPWPSCGGSLEIDDPPQDYPQCMNKALTNNRIIPWARCRVDRPAIYNFTDQQEVLQDWSAQLDNCQGGANTEQQLTVGLWRVPSGPFQPMIWKLMVAMGGIKRIGVGPDSGLEGNITACVPQPYLLLLGGFEIDYENGTRQFNVTCDNCTLSNCVKFGYQNVLIIHQPSFVMLPVNLSEPWYAERGLQILESLNGLLKREKRVAGMIVAGIVTFIAFLISLSTAVVALSQSVQTAAFVDNLSMNVSQALELQEDIDIKMEEKLNALFDTVIYLGRSLEALKHRNHLQCHGEYRWICVTSLAYNMTDYNWQMVQDHLKGAWSHGNETSDLLKLHQQILSIQQAGRISLSPAQVAQSLYDHLKTLVPSSFFAKQIVRGLGLLAVGLLLMFLFFVIFSQYVRSMFLTLAVQLHYQKLITSKRPVSQ

SEQ ID NO: 39 - protein, 665 aa, Monodelphis domestica
MERSGLPVSISNRGRDRFQMQGEQRNRLTSLAPHRLPETRTRSRRASLLSPVSYLMVKRRAPGSKATLVNARTLNTDITFTLMAFTILTLLNICHAEVYWSIVPKPPILRMLTWMDKPPLVYVNNTDWLPHPILAKRVPLESEGALYNYSGSTVYPPFCMSFRNSFIGNSFCVPRRHQAWIVKNATDNSYVGVGMGVIGMGDELARKTFQPKHPANKYLCPNQIGTVQKELPWTDCSVRVPRKVKMPATTDFNIYNWAPRLRCGRSEQLTRLDKYTDYEKWHMPCQNFQKIEDLLEVDMAGSRLAIEAGPIQNTQWKIVAATQPIYKFKVKVKNSGLDLEYDSHINVTACVFAPYVMLVGNNLRIFKNDKGLYEINCTKCRLHQCLSPKHQDSYVAIMYHPPLMWVPVNLTETWASTPTVHIVQKAFNMVIHRSKRMVFALVGAVVSVIAMITSLTTATLALTSSIQNHEFIQEVVSNSSKLWHTQQSIDLAYRDELDSLKDAVFWLGKEVEALHTRITYPCHYNQTGYCITPLPYDNVSYEWQLVVQHIKGAWGSHNSTLDIIKLQQQINKLDQIVYENEAANIAANWEATLSSLDPQNWFGRANLTSIGTIILFILLAIGLIILCSRSCKNRAYIRGILPELARSYHTRQLVPENVELNTTVL

SEQ ID NO: 40 - protein, 689 aa, Rattus norvegicus
MPNHQFGSQTDSSGQPKETDIRPPRQKRPNRRIMRNLAKKIKATRLEPKIKPRQTPTWTQLKTLTAQAKEVLKAAHAPETATLLFVAMLAVMSAPTALGESCWAYLPKPPILHPVGWEVSDHIKVLTNQTLVIGGSPDFHLLKNSSGYVDFEGKSDSLPICFSFSPSVPVGCFQVGKRVFNTDTPTLDNTKLDGKGNGRRMWELWMTTVSDKEEKRQHYTTAKNLPPRYPHYRTTFKKDALWEGDENAFPRWLPCAFPDNGVRFSPLGAAGLLWDFSMSSPDKDQNNYLSTNSKLYGNYVPPVGKPIKRWYDAGWVEPTWFWEPLHTDLPDRQNKPLIEHPELFRLVASSEKLLLKKPGAADYDAVSVSACVTYPYTILVGLPQLINIDKVESIFSIKCTSYKLTNYIDSTVSGSVILIVRRPPYVLLPVDLGDEPWFDDSAIQTIRYATGLIRAKRFVAALILGITALIAIVTSFAVSTTALVKEMQTATFVNDLHKNVTLTLSEQKIIDLKLETRLNALEEVVLELGRDVTNIKTRMATRCHANYDFICVTPLPYNATEEWERTKTHLLGIWDDDNISYNIQELTSLITEMSKQHVDAVDLSGLTRSFADGVKSLNPIDWTQYFIFIGVGILLLVVVLMIFPIVFRCFTDSIAQVQTDLNLVLLKNKKGGTAVPTAEITELRDKHDA

SEQ ID NO: 41 - protein, 690 aa, Macaca mulatta
MNPSEMQRKAPPRRQKHRNRAPLTRMMNQVMISEEQMKSPRTKKAELPTWAQLKKLTPLAGKSLASTKVTQTPEKMLLTALMIVSTVVSLPMPAGAAAANYTYWAYVPFPPLIRAVTWMDNPIEVYVNNSVWVPGPTDDRCPAKPEEEGMMINISIGYRYPPICLGRAPGCLMPAIQNWLVEVPTVSPTSRFTYHMVSGMSLKPQVNYLQDFSYQRSLKFRPKGKPCPKEISRESKDLVWEECVADSAVILQNNTFGTVIDWAPRGQFYHNCTGQTQFCPSALVSPTVDSDLTENLDKHKHKKLQSFYPWIWGEKGISTPRPKMISPVFGPEHPELWRLTVASYRLRIWSGNQTIETRDYKPFYSINLNSSLTVPLQSCVKPPYMLVIGNIVIKPDSQTITCENCRLFTCIDSTFDWQHRILLVRAREGVWIPVSMDRPWEASPSIHILTEVLKGVLSRSKRFIFTLIAVIMGLIAVTATATVAGVALHSSVQTVSFVDNWQKNSTRLWNSQSGIDQKLANQINDLRQTVIWMGDRLMSLEHRFQLQCDWNTSDFCIIPQVYNESKHHWDMVRRHLQGREDNLTLDISKLKEQIFEASQSHLNIVPGAEALDQVAKNLYELNPTTWIKSIGNSTAINFGIMCLCLIGLFLVCWTSRRILRQNRENEQAFIAMAHLYRGKGRENVAGSQGP

SEQ ID NO: 42 - protein, 577 aa, Condylura cristata
MLKVARTFLLLRMLIIVPSASLDNYFWAITRTWPTPFPVHNESLVLPQFYATSCTYGTPCNPKPTSIQEEKFNASRFSLAGTLCFSVALNATDCILLSATNLSVFADPLLHLDPGLSASVLVSALTQAAAGKTQGSGSANGTSDAVNVTTFLVNVSAITPNLISTSENWTFNRSYPLTACRKTVLTFPPEFADCRDTRRPAQKDGWALWPSRFGRWKEKRLIADHPLSAWVLQNVKGATCDLSPFIHLQPSTIHLSNVTGCLNTTSKKLTLHTHRLKDNFTSPGGVVCVDPPFLFLLFNASSESSIDCTTTNCLLSQCWRKTHTYALSLRLPTFVPLPVQANPQDFPVTALIRSRCDFGLTAALVAAIAASIGAAAAAAVAMQTSVQTVAVVNEVIQKTSSSLQTQERLNRHLTSGILLLNKRIDLKSMESNVLTLFDIVSLSCVAKTPHMCVTPYYASLNESRRLSSLLAGNWTREFECLQNNFTAQIVALNMTKAEVVTVRQFTDWLWSSFSFFKEWVGVGMFAAVLVAGLLLLVFLWFRTVRAHRRDKAAITRALIAMDAGHSPQAWISLLREA

(19) United States
(12) Patent Application Publication
(10) Pub. No.: US 2025/0144238 A1
(43) Pub. Date: May 8, 2025

(54) MODULAR BETARETROVIRUS RECEPTOR ATTACHMENT AND CELL ENTRY PROTEINS

(71) Applicant: THE TRUSTEES OF BOSTON COLLEGE, Chestnut Hill, MA (US)

(72) Inventors: Welkin E. Johnson, Lexington, MA (US); Laura R. Hall, Saint Cloud, FL (US); Casey Roberts, Brighton, MA (US); Zachary Williams, Allston, MA (US)

(21) Appl. No.: 18/941,860

(22) Filed: Nov. 8, 2024

Related U.S. Application Data
(60) Provisional application No. 63/597,134, filed on Nov. 8, 2023.

(51) Int. Cl. A61K 48/00; A61K 45/06; C07K 14/005; C12N 7/00; C12N 9/12; C12N 15/86

(52) U.S. Cl. CPC A61K 48/0041; A61K 45/06; C07K 14/005; C12N 7/00; C12N 9/1276; C12N 15/86; C12N 2740/12022; C12N 2740/12023; C12N 2740/12043; C12N 2740/12051; C12Y 207/07049

(57) ABSTRACT

The disclosure relates to betaretroviruses (genus Betaretrovirus), and recombinant betaretroviral envelope (rENV) protein for use in viral vector platforms, an engineered delivery vehicle and gene therapy, their associated methods of use and manufacture. In particular, the present disclosure provides compositions and methods to tune betavirus envelope proteins such that they target a particular cell surface receptor; i.e., are cell and/or tissue-type specific, allowing for better site-specific transgene delivery and expression.

### CROSS-REFERENCE TO RELATED APPLICATIONS

This application claims benefit of priority from U.S. Provisional Application No. 63/597,134, filed on Nov. 8, 2023, titled: Modular Betaretrovirus Receptor Attachment and Cell Entry Proteins, the entire contents of which are incorporated by reference herein for all purposes.

### SEQUENCE LISTING

This application contains a computer readable Sequence Listing which has been submitted electronically in XML format with this application, and is incorporated herein by reference in its entirety. The Sequence Listing XML file submitted with this application was created on Dec. 4, 2024; is named “BCU0022US3.xml” and is 97,458 bytes in size. The Sequence Listing does not go beyond the disclosure in the application as filed.

### BACKGROUND

### Field of the Disclosure

The present disclosure relates to betaretrovirus-based compositions and methods for “tuning” betavirus ENV proteins such that they target a particular cell surface receptor; i.e., are cell and/or tissue-type specific, allowing for better site-specific transgene delivery and expression.

### Background Information

Precise targeting is widely recognized as one of the most significant barriers to the manufacturing and use of viral vectors for gene therapy applications. See Gene therapy coming of age: Opportunities and challenges to getting ahead published Oct. 2, 2019, by E. Capra, J. Smith, and G. Yang, incorporated herein by reference. Specifically, they state that more precise targeting can reduce potential off-target effects and improve safety, which can be especially important if the therapy integrates into or edits the genome.

Currently, a number of viral approaches are in clinical development for gene therapy approaches, including, e.g., adenovirus (AV), adeno-associated (AAV), and Lentivrus (LV). Due in part to the epidemiology of AVs in the human population and AV antigen-specific T cells, which result in the lifelong immunity, vectors based on AV tend to have compromised potencies and trigger a stronger immunological response compared to other viral vectors, such as those based on AAV.

AAV has received much attention because it lacks the essential genes needed for replication and expression of its own genome, making its safety profile attractive but additional challenges exist. Vectors based on AAV are irrevocably limited to a ˜5 kb capacity, so all components needed for proper expression need to be modified to fit into the small capsid. AAV serotypes or engineered capsids have the capacity to transduce multiple tissue/cell types, cell-type-specific, and therefore, transgene expression is by and large controlled at the level of gene transcription. However, innate immunity can detrimentally affect AAV-mediated transgene expression.

Retrovirus infection of a cell begins when the trimeric envelope protein (ENV) complex on the surface of the virion binds to a cognate receptor on a suitable host cell (FIG. 1). Receptor binding leads ultimately to fusion of the virion and cellular membranes, allowing release of the viral capsid core, which contains the viral genome, into the host cell. This process can take place at the plasma membrane or, in the case of some retroviruses, binding results in endocytosis of the bound virion and membrane fusion takes place within the endosome-usually in response to a pH-triggered change in the ENV structure. Following entry, the viral genome is reverse-transcribed into double-stranded DNA and inserted into the chromosomal DNA of the host cell.

ENV is the product of the retroviral env gene. The ENV precursor protein is cleaved by cellular furins, such that the mature ENV comprises two subunits: a surface (SU) subunit and a transmembrane (TM) subunit. SU contains the viral receptor-specificity determinants, while TM comprises a type-I fusion complex that is required for virion-cell membrane fusion. Interactions between SU and TM ensure that receptor binding precedes triggering of fusion with the host cell, while additional interactions within and between subunits are involved in proper assembly of the mature, heterotrimeric spike (SU3-TM3). While superficially similar, retroviruses of each genus differ in the specific mechanisms that regulate the fusion and entry processes. For decades, the focus was on oncogenic retroviruses in the Alpharetrovirus and Gammaretrovirus genera; with the emergence of HIV-1 and AIDS, the focus switched to the Lentivirus genus.

Thanks to the modularity of retrovirus genomes, virions can be engineered to package, reverse-transcribe, and insert non-viral sequences into host-cell DNA, a feature that has been exploited in the development of numerous gene-therapy platforms—the most notable examples being the HIV-1 (genus Lentivirus) and MLV (genus Gammaretrovirus).

Lentiviral vectors have several features that make them amenable to transgene delivery for therapeutic purposes. Lentiviral vectors are integrating vectors that permit long-term transgene expression. They have a packaging capacity of up to 9 kb. High-level expression of multiple genes may be a requisite for achieving therapeutic outcomes for certain diseases. However, an obvious safety consideration in designing a lentiviral vector system for gene therapy is the unintended generation of replication-competent provirus. Also, it has been reported that even self-inactivating lentiviral vectors with strong promoter and enhancer elements can activate neighboring genes, integrate to form chimeric gene fusions, and cause aberrant splicing of cellular transcripts.

Therefore, there exists an ongoing need for improved viral delivery of transgenes for gene therapy approaches. In contrast to AV, AAV and Lentivirus, little is known about betaretroviruses (genus Betaretrovirus), and they have not been widely examined for their potential use in viral vector platforms, and gene therapy. Accordingly, betaretroviruses exist as a potential alternative to currently available viral-based gene delivery platforms.

### SUMMARY

An aspect of the present disclosure relates to betaretroviruses (genus Betaretrovirus), and a recombinant betaretroviral envelope (rENV) protein for use in an engineered delivery vehicle and gene therapy, their associated methods of use, and manufacture. In particular, the present disclosure provides compositions and methods to tune betavirus ENV proteins such that they target a particular cell surface receptor, i.e., are cell and/or tissue-type specific, allowing for better site-specific transgene delivery and expression.

The present disclosure also provides a nucleic acid comprising a polynucleotide that encodes a transcription unit for a recombinant betaretroviral envelope protein comprising a receptor binding domain (RBD), a betaretrovirus surface subunit domain, and a betaretrovirus transmembrane (TM) domain. The RBD is a receptor binding ligand (RBL), wherein the RBL is a peptide, polypeptide or protein ligand binding specifically to a receptor on a cell, a tissue and/or a mammal.

An additional aspect of the present disclosure is directed to an engineered delivery vehicle that comprises a polynucleotide encoding one or more recombinant betaretroviral envelope proteins comprising a receptor binding domain, a betaretrovirus surface subunit (SU) domain, and a betaretrovirus transmembrane domain for forming a packaged nucleic acid vector delivery vehicle.

Another aspect of the present disclosure is related to an engineered delivery vehicle that comprises a virus-like particle having a recombinant betaretroviral envelope (rENV) protein on its surface and including a packaged nucleic acid viral vector cargo. The rENV protein comprises a receptor binding domain (RBD), a betaretrovirus surface subunit (SU) domain, and a betaretrovirus transmembrane (TM) domain. The packaged nucleic acid viral vector cargo comprises a polynucleotide encoding the rENV, and a transcription unit encoding a therapeutic protein.

The present disclosure also provides an engineered delivery vehicle that comprises a polynucleotide encoding one or more betaretroviral elements or recombinant betaretroviral envelope proteins for forming the engineered delivery vehicle for packaging cargo within the delivery vehicle. In some embodiments, the betaretroviral elements or recombinant betaretroviral envelope proteins comprise a betaretroviral envelope protein, which comprises at lease one of a surface subunit (SU) domain, a betaretrovirus transmembrane (TM) domain or a combination thereof. In any aspect or embodiment described herein, the SU domain comprises a recombinant receptor binding domain (RBD) and a scaffold domain. In any aspect or embodiment described herein, the SU domain is covalently or non-covalently bound to the TM domain.

In any aspect or embodiment described herein, the RBD is a receptor binding ligand (RBL), which is a peptide, polypeptide, or protein ligand binding specifically to a receptor. In some embodiments, the RBD binds specifically to a receptor on a target cell and/or target tissue.

In any aspect or embodiment described herein, the betaretroviral envelope protein is from a mouse mammary tumor virus (MMTV), a jaagsiekte sheep retrovirus (JSRV), or an intracisternal a-type particle elements with an envelope (IAPE).

In any aspect or embodiment described herein, the cargo is a transgene encoding a therapeutic protein, which comprises an immune checkpoint modulator, an antibody or portion thereof, a fusion protein, an anticoagulant, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, an antiviral, and any combination thereof.

In any aspect or embodiment described herein, the engineered delivery vehicle comprises a reverse transcriptase, and the engineered delivery vehicle is a virus-like particle.

In another aspect, the present disclosure provides a method of delivering cargo to a target cell and/or target tissue by contacting the target cell and/or the target tissue with a polynucleotide encoding one or more betaretroviral elements or recombinant betaretroviral envelope proteins for forming an engineered delivery vehicle for packaging cargo within the delivery vehicle.

In any aspect or embodiment described herein, the method further comprises contacting the target cell and/or the target tissue with a reverse transcriptase. In some embodiments, the delivery vehicle is a virus-like particle.

In any aspect or embodiment described herein, the target cell is a mammalian cell and the tissue is a mammalian tissue. In some embodiments, the target cell is Crandell-Rees Feline Kidney (CRFK) cell, Vero cell, or human embryonic kidney 293T cell.

In any aspect or embodiment described herein, the polynucleotide, which is delivered to the target cells and/or the target tissue by the method described herein, encodes one or more betaretroviral elements or recombinant betaretroviral envelope proteins for forming a delivery vehicle for packaging cargo within the delivery vehicle. In some embodiments, the betaretroviral elements or recombinant betaretroviral envelope proteins comprise betaretroviral envelope protein, which comprises a surface subunit domain and a betaretrovirus transmembrane domain. In any aspect or embodiment described herein, the SU domain comprises a recombinant receptor binding domain. In any aspect or embodiment described herein, the SU domain comprises a scaffold domain and a recombinant receptor binding domain. In any aspect or embodiment described herein, the SU domain is covalently or non-covalently bound to the TM domain. In any aspect or embodiment described herein, the RBD is a receptor binding ligand (RBL), which is a peptide, polypeptide, or protein ligand binding specifically to a receptor. In some embodiments, the RBD binds specifically to a receptor on a target cell and/or target tissue.

In any aspect or embodiment described herein, the method further comprises contacting the target cell and/or the target tissue with a reverse transcriptase.

An additional aspect of the present disclosure is a method of treating, preventing, or ameliorating a genetic disease, disorder, or syndrome in a subject in need thereof, comprising the step of administering to the subject a therapeutically effective amount of a polynucleotide that encodes one or more betaretroviral elements or recombinant betaretroviral envelope proteins for forming a delivery vehicle for packaging cargo within the delivery vehicle, wherein the polynucleotide treats or ameliorates at least one symptom of the genetic disease, disorder, or syndrome.

In any aspect or embodiment described herein, the method further comprises determining whether the cells or tissues of the subject express the receptor of interest by any assay or method for detecting a DNA or RNA sequence. In some embodiments, the assay or method comprises genotyping, genome sequencing, next generation sequencing, and gene expression analysis of a sample obtained from the subject.

In any aspect or embodiment described herein, the method further comprises administering a therapeutically effective amount of reverse transcriptase to the subject in need thereof. The reverse transcriptase could be encoded by a polynucleotide. The reverse transcriptase could be encoded by the polynucleotide encoding one or more betaretroviral elements or recombinant betaretroviral envelope proteins for forming an engineered delivery vehicle for packaging cargo within the delivery vehicle.

In any aspect or embodiment described herein, the polynucleotide, which is delivered to the subject in need thereof by the method, encodes one or more betaretroviral elements or recombinant betaretroviral envelope proteins for forming a delivery vehicle for packaging cargo within the delivery vehicle. In some embodiments, the betaretroviral elements or recombinant betaretroviral envelope proteins comprise betaretroviral envelope protein, which comprises a surface subunit domain and a betaretrovirus transmembrane domain. In some embodiments, the SU domain comprises a recombinant receptor binding domain and a scaffold domain. In some embodiments, the SU domain is covalently or non-covalently bound to the TM domain. In some embodiments, the RBD is a receptor binding ligand, which is a peptide, polypeptide or protein ligand binding specifically to a receptor. In some embodiments, the RBD binds specifically to a receptor on a target cell and/or target tissue.

In any aspect or embodiment described herein, the method involves administering an effective amount of polynucleotides encoding betaretroviral elements, betaretroviral envelope protein, or a recombinant betaretrovirus, or a recombinant betaretroviral envelope protein to a subject. In any aspect or embodiment described herein, the subject is a human, non-human primate, dog, cat, livestock (e.g., cattle, sheep, pig, goat, horse, donkey, mule, buffalo, oxen, llama, camel, alpaca, cow, ox, reindeer, sheep, water buffalo, yak, et.), poultry (e.g., chicken, turkey, etc.).

In some embodiments, the subject is a human, non-human primate, dog, cat, livestock, or poultry. In some embodiments, the betaretroviral envelope protein is from a mouse mammary tumor virus, a jaagsiekte sheep retrovirus, or an intracisternal a-type particle elements with an envelope.

In some embodiments, the disorder is hypercholesterolemia, and the RBL is a cyclic 8-residue peptide (VH434), specifically binding to low-density lipoprotein (LDL) receptors.

The details of one or more embodiments of the present disclosure are set forth herein. Other features, objects, and advantages of the present disclosure will be apparent from the Detailed Description, Examples, Figures, and Claims.

### BRIEF DESCRIPTION OF THE DRAWINGS

The accompanying drawings, which are incorporated into and form a part of the specification, illustrate several embodiments of the present disclosure and, together with the description, serve to explain the principles of the present disclosure. The drawings are only for the purpose of illustrating specific embodiments of the present disclosure and are not to be construed as limiting the present disclosure. Further objects, features, and advantages of the disclosure will become apparent from the following detailed description taken in conjunction with the accompanying figures showing illustrative embodiments of the disclosure.

FIG. 1. Illustrates a retrovirus replication cycle. HIV-1 replication is depicted as an example. ENV complexes (spikes) on the virion surface mediate attachment to a specific cell surface receptor, which ultimately leads to fusion of the virion and cell membranes and release of the viral capsid core (containing the viral RNA genomes) into the cell. Subsequent steps result in integration of a dsDNA copy of the viral genome into the host cell chromosomal DNA; the resulting provirus is then expressed, giving rise to new viral genomes and proteins, which assemble into progeny virions and bud from the cell membrane.

FIG. 2. Betaretroviruses use diverse, unrelated proteins as cellular receptors. Only three receptors have been identified to date, for IAPE, JSRV and MMTV. Although a cellular receptor for HERV-K (HML2) ENV has not yet been identified, the three known receptors have been ruled out.

FIG. 3. Divergent betaretroviral SU sequences share a common set of short, highly conserved motifs, which form the hypothetical framework regions (FRs). FRs are separated by sequence- and length-variable regions (VARs) which have low identity/similarity between viruses. Alignment includes MMTV, JSRV, HERV-K (HML2) and a dozen additional ERV-encoded betaretrovirus-like ENV proteins from diverse mammalian genomes, representing hundreds of millions of years of betaretrovirus evolution.

FIG. 4. AlphaFold predicts betaretrovirus SU comprises two domains, one comprising the FRs (blue) and one comprising the major VARs (pink/dark pink). Examples shown are the SUs of HERV-K (HML2), MMTV and JSRV. Note the FR domains take on a similar topology in all three structures, while the VAR domains differ significantly between the three viruses. These observations suggest that the FR domain plays an essential, functionally conserved role, and that the VAR domains are likely the primary determinants of receptor-specificity, which differs between the three viruses.

FIG. 5. Low sequence identity and high structural similarity of betaretrovirus SU domains. Left: NJ tree based on aligned amino-acid sequences of SU domains. Right: heatmap depicting % protein sequence identity (below diagonal) and pairwise RMSD scores (above diagonal). For example, HERV-K Phoenix SU and SERV-K1 SU have both high sequence identity and high structural similarity (red boxes both below and above the diagonal). L. Africana ERV SU and Horse ERV SU have moderate sequence similarity (blue box below the diagonal) but high structural similarity (red box above the diagonal). However, the majority of pairwise comparisons have low sequence identity (violet-purple boxes below the diagonal) but high structural similarity (orange-red boxes above).

FIG. 6. AlphaFold2 predicted SU structures fall into three classes. White arrowheads indicate a central alpha-helix that separates variable (upper/apical) and conserved (lower) domains. J-class. From left to right: JSRV, bovine ERV1, canine ERV, naked mole rat ERV, and overlay. Naked mole rat SU has features consistent with J- or M-class. M-class. From left to right: MMTV, rat ERV, bovine ERV-K3 (fematrin), elephant ERV, and overlay. K-class. From left to right: HERV-K (HML2), Bovine retrovirus Ch15, Bovine ERV-K2, Opossum ERV, and overlay. Not shown-IAPE may represent a fourth class (I-class).

FIG. 7. Betaretrovirus ENV as a platform for customized receptor targeting. All betaretrovirus ENV proteins form a heterodimer of SU and TM. TM comprises the highly conserved fusion machinery. SU contains the receptor-binding determinants, and SU must interact with TM to form stable heterotrimers. Betaretrovirus SU structures accommodate diverse RBDs. Our results indicate that SU comprises two domains, one highly conserved “scaffold” and the other a highly variable “receptor-binding domain” (RBD). It is believed that the “scaffold” portion physically links the functions of the RBD and the conserved, fusogenic functions of TM. This suggests that in nature, distinct RBDs can work with the same or similar scaffolds, which in turn links them to a common fusion apparatus to facilitate viral entry. This structural flexibility provides a means to rationally design novel betaretroviral ENVs to target pre-specified receptors, as a strategy to circumvent the specificity issues with current gene therapy platforms. Because potentially hundreds or thousands of betaretroviral ENV sequences are available (as mammalian ERVs), scaffolds can be pre-selected from a library of structures based on predicted compatibility with the ligand(s) of interest via in silico modeling modeling of engineered proteins, or by screening structures for structural and chemical similarities to the ligand of interest.

FIG. 8. Confirmation of divergent betaretroviral ENV proteins among ERV loci in vertebrate genome assemblies in cell culture. ERV loci with bioinformatically predicted ORFs encoding putative betaretrovirus ENV proteins were used to generate synthetic expression constructs, including an in-frame, C-terminal Avi-tag. These were assessed for expression by transient transfection and western blot of cell lysates. The figure depicts several such ENVs. Lysates in lanes with a “+” were pre-treated with PNGaseF to remove mature glycans. The blots confirm that all but one of these are expressed at the predicted sizes for the full length, glycosylated precursors (SU-TM), and that they are furin cleaved to produce TM subunits of the predicted size. One, Bovine ERV1, was expressed but not cleaved, and site-directed mutation to correct the furin cleavage site resulted in an unstable or poorly expressed protein. With this exception, expression and processing are consistent with expectations for betaretrovirus ENV proteins. (IAPE-D1, mouse ERV; BoRV CH15, bovine betaretrovirus CH15; SERV-K1, rhesus macaque ERV-K; HERV-K, human ERV-K; MMTV, mouse betaretrovirus; BoERV3, bovine ERV 3; BoERV1, bovine ERV-1; BoERV1 L382R, bovine ERV-1 with a modified furin cleavage site).

FIG. 9. Cell-cell fusion mediated by endogenous retrovirus (ERV) encoded ENVs. Two cell lines stably expressing different halves of a split GFP are mixed and transfected with ENV expression constructs. If the ENV mediates cell-cell fusion, GFP multimers form and fluoresce, usually within minutes of cell fusion. Cells were either left at neutral pH (upper two rows) or pulsed with low pH (lower two rows). “No Env” was an empty vector that served as a negative control, and VSV-G served as a positive control. BabFCdelta22 ENV is an ERV-encoded ENV from the baboon genome with a truncated cytoplasmic domain that enhances fusogenicity; HERV-K Ph is a previously reported human ERV ENV, HERV-K (HML2); HERV-K Phdelta659-699 is the same ENV with a truncation of the cytoplasmic domain that increases fusogenicity. SERV-K1 ENV is a betaretrovirus ENV encoded by an ERV in the rhesus macaque genome. As expected, fusion by the three betaretrovirus ENVs was pH dependent (last three columns, compare upper and lower panels).

FIG. 10. Synthetic ENV is compatible with a lentivirus vector system, and the combination can be used to deliver a genetic “cargo” or “payload”. Lentivirus vectors can be pseudotyped with ERV-derived betaretrovirus ENVs and deliver a reporter to feline, monkey and human cell lines (CRFK, Vero and 293T cells, respectively). 293T cells were cotransfected with a lentiviral vector encoding the viral structural proteins and a transducible GFP reporter and an ENV expression construct. Supernatants were harvested 48-72 hours post-transfection and used to infect the indicated cell lines. VSV-G pseudotyped vector was used as a positive control. Hep-2 cells are known to be refractory to HERV-K mediated infection. Dots indicate biological replicates. * significantly different from mock/background (p<0.01, 0.001, <0.0001, ns=not significant).

FIG. 11. Expression and processing of a betaretrovirus ENV engineered to encode a peptide ligand. AlphaFold was used to predict structures of the JSRV ENV SU subunit wild type (left) or with insertions in the 2nd variable domain (V2) of a cyclic 8-residue peptide (VH434, blue and yellow). VH434 is reported to be a ligand for the human LDL receptor (David, M. et al. 2018. PLOS One. 13, e0191052). A version predicted to result in minimal divergence from the parental structure was identified by in silico screening (middle). This sequence was then synthesized and expressed by transient transfection and visualized by SDS-PAGE and western blot (right). The synthetic ENV produces a precursor protein of the expected size (upper band) and a lower band corresponding to the furin-released TM subunit.

FIG. 12. 5 Wild type JSRV ENVwt and JSRV ENVVH434 display similar, low level, pH-dependent fusion in human cells. Upper panels are at neutral pH, lower panels are after a brief, low pH (5.0) pulse. Small syncytia and GFP+ cells indicate membrane fusion. An empty vector served as a negative control (“No Env”). VSV-G served as a positive control. JSRV is an ovine betaretrovirus, and low-level fusion results from suboptimal binding to the human ortholog of the HYAL2 receptor.

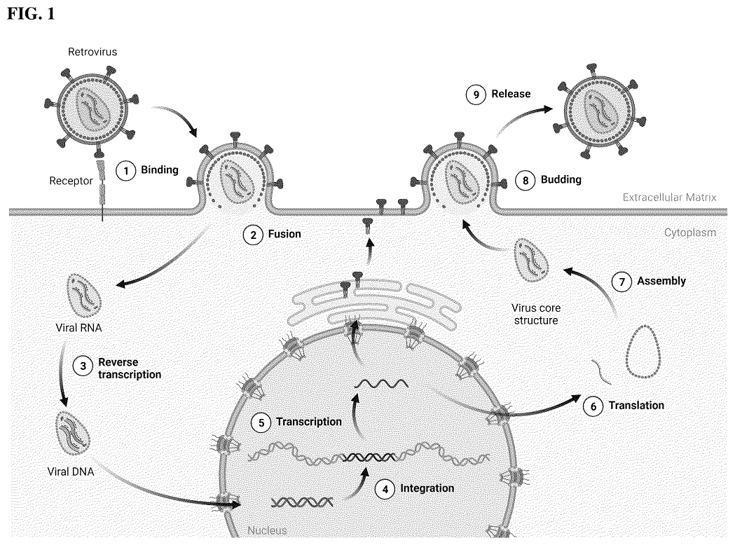

### DETAILED DESCRIPTION

Current gene therapy platforms either result in non-specific delivery, are immunogenic, or require incorporation of multiple, different proteins into each viral particle for the cell-specific binding and entry functions. The latter results in loss of efficiency due to the need to get different proteins to incorporate into the same particle in appropriate ratios. The ideal viral vector system uses a single viral protein that combines both target cell receptor-specific binding and membrane fusion functions. It is also desirable to provide a platform for viral vector development based on a viral protein that can be engineered to use pre-specified receptors of interest, without compromising virion incorporation, fusogenicity, or infectivity.

Retroviruses continue to play a major role in the development of gene therapy vector platforms. This began with early work with murine leukemia virus (MLV, genus Gammaretrovirus) and includes significant investment in developing vectors based on HIV-1 (genus Lentivirus). To our knowledge, very little attention or effort has focused on betaretroviruses. As described previously, our evolution-guided and structure-guided analyses indicates that retroviruses in the genus Betaretrovirus have envelope proteins (ENVs) with sequence and structural properties that are well-suited for solving a major barrier in targeted gene-delivery and gene-therapy applications. Specifically: 1) betaretrovirus ENV proteins exist as natural sequences that exist in thousands of copies in vertebrate genomes in the form of endogenous retrovirus (ERV) loci, 2) comparative evolutionary analyses and AI-guided structure prediction suggests that these have highly divergent receptor-binding domains, but 3) that these also share a highly conserved “core” structure. This means that in nature, a conserved core structure has been coupled to an array of structurally diverse receptor-binding regions. Based on these observations, we predict that synthetic betaretrovirus ENVs can be rationally designed and customized to bind to user-specified cell surface receptors by retaining the conserved core and modifying the variable regions. These can then be coupled with existing gene-delivery platforms, including lentivirus vectors, to achieve cell-specific or tissue-specific payload delivery, ex vivo and/or in vivo.

As described herein, evolution-guided models of betaretrovirus ENV proteins demonstrate that these share a core structure that allows the betaretroviruses to evolve to use different, unrelated receptors. Described herein is a betaretrovirus-based platform that provides similar structural flexibility, allowing for engineering receptor-specific viral entry proteins (referred to herein, as “modular betaretrovirus attachment & cell entry”, or MBRACE).

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein may be employed in practicing the disclosure. It is also understood that every embodiment of the disclosure may optionally be combined with any one or more of the other embodiments described herein that are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

### Definitions

Unless defined otherwise or clearly indicated otherwise by their use herein, all technical and scientific terms used herein have the same meaning as commonly understood by those of ordinary skill in the art to which this application belongs.

As used in the specification and the appended claims, the indefinite articles “a” and “an” and the definite article “the” can include plural referents as well as singular referents unless specifically stated otherwise or the context clearly indicates otherwise.

The term “exemplary” as used herein means “serving as an example, instance or illustration”. Any embodiment or feature characterized herein as “exemplary” is not necessarily to be construed as preferred or advantageous over other embodiments or features.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

The term “in need thereof” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

As used herein, the term “in combination” refers to the use of more than one prophylactic and/or therapeutic agent simultaneously or sequentially and in a manner such that their respective effects are additive or synergistic.

As used herein, the terms “treat,” “treatment,” “treating,” or “amelioration” refer to therapeutic treatments, wherein the object is to reverse, alleviate, ameliorate, inhibit, slow down or stop the progression or severity of a disease, disorder, or syndrome related to a bacteria. The term “treating” includes reducing or alleviating at least one adverse effect or symptom of a disease, disorder, or syndrome. Treatment is generally “effective” if one or more symptoms or clinical markers are reduced. Alternatively, or in addition, treatment is “effective” if the progression of a disease, disorder, or syndrome is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. For example, treatment is considered effective if the condition is stabilized. The term “treatment” of a disease also includes providing relief from the symptoms or side-effects of the disease (including palliative treatment).

As used herein, the term “gene expression” includes both gene transcription, whereby DNA (or RNA in the case of some RNA-containing viruses) corresponding to a gene is transcribed to generate an RNA molecule and RNA translation, whereby an RNA molecule is translated to generate a protein encoded by the gene. As used herein, the term “protein expression” is used to refer both to gene expression comprising transcription of DNA (or RNA) to form an RNA molecule and subsequent processing and translation of the RNA molecule to form protein and to gene expression comprising translation of mRNA to form protein.

As used herein, a “subject”, “patient”, “individual” and like terms are used interchangeably and refers to a host organism that can be an animal, a plant, or a single-cell microbe.

As used herein, the term “administering,” refers to the placement of an agent (e.g., a bacteriophage) as disclosed herein into a subject by a method or route that results in at least partial delivery of the agent at a desired site.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases, such as “comprising,” “including,” “carrying,” “having,” “containing,” “involving,” “holding,” “composed of,” and the like are to be understood/construed to be open-ended (i.e., to mean including but not limited to) unless otherwise noted. Only the transitional phrases “consisting of” and “consisting essentially of” shall be closed or semi-closed transitional phrases, respectively, as set forth in the United States Patent Office Manual of Patent Examining Procedures, Section 2111.03.

The terms first, second, etc., as used herein are not meant to denote any particular ordering, but simply for convenience to denote a plurality of, for example, compositions. All methods described herein can be performed in a suitable order unless otherwise indicated herein or otherwise clearly contradicted by context. Thus, in certain methods described herein that include more than one step or act, the order of the steps or acts of the method is not necessarily limited to the order in which the steps or acts of the method are recited unless the context indicates otherwise.

The articles “a” and “an” as used herein and in the appended claims are used herein to refer to one or to more than one (i.e., to at least one) of the grammatical object of the article unless the context clearly indicates otherwise. By way of example, “an element” means one element or more than one element. The use of the terms “a” and “an” and “the” and similar referents (especially in the context of the claims) are to be construed to cover both the one or more than one (e.g., “at least one”, “plurality”, or “one or more”) of the grammatical object of the article, unless otherwise indicated herein or clearly contradicted by context. By way of example, “an element” means one element or more than one element.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The terms “decrease”, “reduce”, “reduction”, “lower” or “lowering,” or “inhibit” are all used herein generally to mean a decrease by a statistically significant amount. For example, “decrease”, “reduce”, “reduction”, or “inhibit” means a decrease by at least 10% as compared to a reference level, for example a decrease by at least about 20%, or at least about 30%, or at least about 40%, or at least about 50%, or at least about 60%, or at least about 70%), or at least about 80%, or at least about 90% or up to and including a 100% decrease (e.g., absent level or non-detectable level as compared to a reference level), or any decrease between 10-100%) as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The term “polynucleotide”, “nucleic acid”, or “nucleic acid sequence” refers to a polymer composed of nucleotide units. Polynucleotides can contain naturally occurring nucleic acids (e.g., deoxyribonucleic acid [“DNA”] and ribonucleic acid [“RNA”]), or/and nucleic acid analogs. For example, the term “nucleic acid” or “polynucleotide” includes single-, double- or multi-stranded DNA or RNA, genomic DNA, cDNA, DNA-RNA hybrids, or a polymer comprising purine or pyrimidine bases, or other natural, chemically or biochemically modified, non-natural, or derivatized nucleotide base. Polynucleotides containing one or more nucleic acid or nucleotide analogs are sometimes called “aptamers”. Nucleic acid or nucleotide analogs include without limitation those which have a non-naturally occurring base/nucleobase, have a sugar or non-sugar moiety other than 2′-deoxyribose or ribose, or engage in linkages with other nucleotides other than the naturally occurring phosphodiester bond, or a combination thereof. Non-limiting examples of nucleic acid or nucleotide analogs include xeno (biotic) nucleic acids (XNAs) having a backbone other than the naturally occurring sugar-phosphate backbone present in DNA or RNA (e.g., bridged nucleic acids [BNAs], cyclohexene nucleic acids [CeNAs], 2′-deoxy-2′-fluoroarabino nucleic acids [FANAs], glycol nucleic acids [GNAs], 1,5-anhydrohexitol nucleic acids [HNAs], locked nucleic acids [LNAs], 2′-O-methyl ribonucleotides, morpholino nucleic acids [MNAs], peptide nucleic acids [PNAs], and threose nucleic acids [TNAs]), phosphorothioates, phosphorodithioates, phosphorotriesters, phosphoramidates, boranophosphates, methylphosphonates, chiral-methyl phosphonates, and the like. DNA and RNA polynucleotides can be synthesized using a DNA or RNA polymerase or an automated DNA or RNA synthesizer. Polynucleotides containing nucleic acid analogs can be synthesized using, e.g., an engineered DNA or RNA polymerase that recognizes the nucleic acid analogs, a phosphoramidite strategy, or an automated peptide synthesizer in the case of PNAs. The term “nucleic acid molecule” typically refers to a larger polynucleotide. The term “oligonucleotide” typically refers to a shorter polynucleotide. In certain embodiments, an oligonucleotide contains no more than about 50 nucleotides. In some embodiments, when a polynucleotide sequence is represented by a DNA sequence (i.e., A, T, G, C), this also includes the corresponding, or the complementary, RNA sequence (i.e., A, U, G, C) in which “U” replaces “T”.

Conventional notation is used herein to describe polynucleotide sequences: the left-hand end of a single-stranded polynucleotide sequence is the 5′ end; the left-hand direction of a double-stranded polynucleotide sequence is referred to as the 5′ direction. The direction of 5′ to 3′ addition of nucleotides to a newly forming (daughter) DNA strand, or to a nascent RNA transcript, is referred to as the direction of replication or transcription, respectively. The DNA strand having a complementary, antiparallel sequence as a daughter DNA strand or a primary RNA transcript (the “coding strand”) is called the “template strand”. “Upstream” is toward the 5′ end of an RNA molecule, and “downstream” is toward its 3′ end. When considering double-stranded DNA, “upstream” is toward the 5′ end of the coding strand for the gene in question and “downstream” is toward the 3′ end of the coding strand, so the 3′ end of the template strand is upstream of the gene in question and the 5′ end of the template strand is downstream of the gene.

The term “primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as a DNA or RNA polymerase. A primer is typically single-stranded but may be double-stranded. Primers are typically deoxyribonucleic acids, but a wide variety of synthetic and naturally occurring primers are useful for many applications. A primer is complementary to the template to which it is designed to hybridize and serves as a site for the initiation of synthesis but need not reflect the exact sequence of the template. In such a case, specific hybridization of the primer to the template depends on the stringency of the hybridization conditions. A primer can be labeled with an agent that promotes isolation (e.g., biotin), separation (e.g., biotin) or detection (e.g., a fluorescent, chromogenic or radioactive moiety) of the coding strand in single-stranded form or the coding strand hybridized to the template strand.

The term “gene therapy” refers to a therapeutic technique that involves the modification, replacement, or introduction of genetic material into a patient's cells to treat or prevent disease. Gene therapies typically utilize vectors, such as viral or non-viral delivery systems, to transport functional copies of genes, RNA molecules, or gene-editing components to target cells. Once delivered, the genetic material may integrate into the patient's genome, produce therapeutic proteins, or repair existing DNA errors.

The term “cancer disease” or “cancer” includes a disease characterized by aberrantly regulated cellular growth, proliferation, differentiation, adhesion, and/or migration. “Cancer cell” means an abnormal cell that grows by rapid, uncontrolled cellular proliferation and continues to grow after the stimuli that initiated the new growth cease.

“Metastasis” refers to the process by which cancer cells spread from their original site to other parts of the body. This complex phenomenon involves several stages, including the detachment of malignant cells from the primary tumor, invasion into the extracellular matrix, penetration through the endothelial basement membranes to access blood vessels and body cavities, and eventual transport through the bloodstream to infiltrate target organs. The establishment of new tumors at these sites relies on angiogenesis, the formation of new blood vessels. Notably, metastasis can occur even after the primary tumor has been removed, as residual cancer cells may persist and acquire metastatic capabilities. In the context of this invention, “metastasis” specifically refers to “distant metastasis,” which indicates the spread of cancer cells far from the primary tumor and regional lymph nodes.

The term “contacting” refers to the act of making contact or bringing into immediate or close proximity, whether at the cellular or molecular level. This can involve initiating a physiological reaction, chemical reaction, or physical change, and may occur in various environments, such as in a solution, reaction mixture, or in vitro and in vivo conditions.

Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

“Pharmaceutical compositions” are compositions comprising at least one active agent, and at least one other substance, such as a carrier, excipient, or diluent. Pharmaceutical compositions meet the U.S. FDA's GMP (good manufacturing practice) standards for human or non-human drugs.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

A “therapeutically effective amount” means an amount effective, when administered to a patient, to provide a therapeutic benefit such as an amelioration of at least a symptom of the disorder, decrease the frequency or severity of symptoms, or effect a change in a clinical marker for a disease or disorder, slowing the progression of a disease or disorder, halting the progression of a disease or disorder, or reversing the course of a disorder.

The term “virus-like particle” (VLP) refers to a structure that in at least one attribute resembles a virus, but which has not been demonstrated to be infectious. A VLP may be a nonreplicating, noninfectious viral shell that contains a viral capsid but lacks all or part of the viral genome, in particular, the replicative components of the viral genome. VLPs are generally composed of one or more viral proteins, such as, but not limited to those proteins referred to as capsid, coat, shell, surface, and structural proteins.

A “reverse transcriptase” is an enzyme used to generate complementary DNA (cDNA) from an RNA template, a process termed reverse transcription. A reverse transcriptase is used by betaretrovirus to replicate their genomes.

The terms “cargo”, “cargo molecular”, “packaged nucleic acid viral vector cargo” and “payload” are used interchangeably and refer to, e.g., a molecule, agent or compound that is carried or shuttled by another agent to a site or location, and confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition. The cargo may be a transgene encoding a therapeutic protein. The therapeutic protein may be an immune checkpoint modulator, an antibody or portion thereof, a fusion protein, an anticoagulant, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, an antiviral, and any combination thereof.

An “engineered delivery vehicle” and a “packaged nucleic acid vector delivery vehicle” are used interchangeably and comprises one or more components encoded in polynucleotides in the engineered delivery system described herein. As described elsewhere herein, such components include, but are not necessarily limited to, polynucleotides encoding one or more betaretroviral elements, betaretroviral envelope proteins, or recombinant betaretroviruses, or recombinant betaretroviral envelope proteins for forming a delivery vehicle, or an engineered delivery vehicle for packaging cargo within the delivery vehicle and deliver the cargo into to target cells and tissues.

A genetic “element” within a vector refers to specific nucleic acid (e.g., DNA, RNA), nucleotide, polynucleotide, open reading frame (ORF), or framework region (FR) that play distinct roles in gene delivery, expression, or regulation of gene(s). Representative genetic “element” includes promoters, enhancers, polyadenylation (poly-A) signals, splicing sites, replication origins and selectable markers.

A “transcription unit” refers to specific nucleic acid (e.g., DNA, RNA), nucleotide, polynucleotide, open reading frame (ORF), or framework region (FR) that play distinct roles in gene delivery, expression, or regulation of gene(s). Representative genetic “transcription unit” includes promoters, enhancers, polyadenylation (poly-A) signals, splicing sites, replication origins and selectable markers.

A “betaretroviral element” refers to a specific sequence (e.g., polynucleotides, polypeptide and polynucleotides encoding them) derived from betaretroviruses that enables the vector to deliver and integrate genetic material into the host genome. The sequence comprises nucleic acid, nucleotide, polynucleotide, open reading frame (ORF), or framework region (FR) derived from betaretroviruses. The sequence also comprises amino acid or polypeptide encoded by nucleic acid, nucleotide, polynucleotide, open reading frame, or framework region derived from betaretroviruses. Representative betaretroviral elements or recombinant betaretroviral envelope proteins include but are not limited to betaretroviral envelope (ENV) protein, surface subunit (SU) domain of envelope protein, scaffold domain of envelope protein, transmembrane (TM) domain of envelope protein, viral receptor-specificity determinants of SU domain, type-I fusion complex of TM domain, and receptor binding domains (RBDs), signal peptide, glycosylation sites, furin cleavage site, fusion peptide, heptad repeats, membrane spanning region, cytoplasmic domain.

The engineered polynucleotide may further include regulatory elements to control expression of the engineered delivery vehicle. The term “regulatory element” refers to promoters, enhancers, Kozak sequences, Internal Ribosome Entry Sites (IRES), other expression control elements and cellular localization signals. Such regulatory elements are described, for example, in Goeddel, Gene Expression Technology: Methods In Enzymology 185, Academic Press, San Diego, Calif. (1990).

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Immunology by Werner Luttmann, published by Elsevier, 2006. Definitions of common terms in molecular biology can also be found in Benjamin Lewin, Genes X, published by Jones & Bartlett Publishing, 2009 (ISBN-10:0763766321); Kendrew et al. (eds.), Molecular Biology and Biotechnology: A Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, Ist edition, 1998) which are all incorporated by reference herein in their entireties.

Viral vectors are the focus of intensive research and development in both the academic and biotech sectors, primarily because of the hope that gene therapy can be used to correct a wide spectrum of inherited and somatic genetic disorders. Targetable viral vectors can also be useful tools to research in organismal model systems, where precise delivery of genetic modifications is desirable in order to study or understand developmental processes. Although viral vectors are widely used for such applications, these suffer from a lack of precise targeting, and often have to be re-engineered for each novel application.

Many viruses use cell surface receptors to bind to host cells and initiate fusion between the virion membrane and the host cell membrane, resulting in delivery of the virion contents into the host cell. This natural process is the cornerstone of many approaches to gene therapy. Current viral vectors used for gene delivery (e.g., for gene therapy) rely on the natural receptor specificities of well-studied viruses; prominent examples include HIV-1 and MLV (retroviruses), VSV (a rhadbdovirus), and AAV (a parvovirus). Depending on the tissue distribution of the virus' receptor, infection can result in delivery of the payload into many irrelevant cell types and tissues, in addition to the cell type or tissue of interest. Conversely, a cell or tissue target of choice may not express a receptor conveniently used by any known viruses for entry, precluding use of a viral delivery system for treatment. To solve these issues, significant modifications of existing viral vector platforms must be used; however, these often reduce the efficiency of vector production and result in diminished delivery capacity (infectivity) of the engineered viral particles.

The ideal viral vector is one that can be rationally designed and modified to use pre-specified receptors, based on the target cell or tissue type and the nature of the disease or condition being treated. To construct a model of betaretrovirus entry proteins and understand how they evolved to use different receptors, betaretrovirus Env proteins and related endogenous retrovirus (ERV) sequences found abundantly in mammalian genomes that encode betaretrovirus-related proteins were used. The resulting model suggests that all betaretrovirus entry proteins evolved from a common core structure. This structure provided evolutionary flexibility, which allowed the betaretroviruses to spread to many different mammalian hosts and to adapt to different cellular receptors. Taking a cue from nature, we proposed that this evolutionary flexibility also means that betaretrovirus entry proteins can be customized in the laboratory to recognize pre-specified receptors. We refer to this as modular betaretrovirus attachment & cell entry (MBRACE).

An aspect of the present disclosure relates to betaretroviruses (genus Betaretrovirus), and betaretrovirus ENV proteins for use in viral vector platforms, an engineered delivery vehicle and gene therapy, their associated methods of use and manufacture. In particular, the present disclosure provides compositions and methods to tune betavirus ENV proteins such that they target a particular cell surface receptor; i.e., are cell and/or tissue-type specific, allowing for better site-specific transgene delivery and expression.

The advantage of MBRACE is the ability to customize a single viral protein for precise delivery across multiple applications. This will increase the efficiency of production, and reduce some of the burden of repeated, iterative evaluation processes incurred for each new application.

In an aspect, the compositions and methods described herein are used for rational design of viral entry proteins to use pre-specified receptors for cell entry, for example, receptor/biomarker-specific targeting of viral vectors for gene therapy. In certain embodiments, the compositions and methods are useful for ex vivo, in vitro, as well as in vivo gene delivery.

Thus, in an aspect, the description provides a recombinant betaretrovirus vector or betaretrovirus ENV protein as described herein.

In any aspect or embodiment described herein, the recombinant betaretrovirus vector as described or claimed herein comprising expressing a recombinant betaretrovirus ENV protein and a transgene, and the transgene encodes a therapeutic protein.

In any aspect or embodiment described herein, the disclosure provides a nucleic acid comprising a polynucleotide that encodes a transcription unit for a recombinant betaretroviral envelope protein comprising a receptor binding domain (RBD), a betaretrovirus surface subunit domain, and a betaretrovirus transmembrane (TM) domain. The RBD is a receptor binding ligand (RBL), wherein the RBL is a peptide, polypeptide or protein ligand binding specifically to a receptor on a cell, a tissue and/or a mammal.

In any aspect or embodiment described herein, the disclosure provides an engineered delivery vehicle that comprises a polynucleotide encoding one or more recombinant betaretroviral envelope proteins comprising a receptor binding domain, a betaretrovirus surface subunit (SU) domain, and a betaretrovirus transmembrane domain for forming a packaged nucleic acid vector delivery vehicle.

In any aspect or embodiment described herein, the disclosure provides an engineered delivery vehicle that comprises a virus-like particle having a recombinant betaretroviral envelope (rENV) protein on its surface and including a packaged nucleic acid viral vector cargo. The packaged nucleic acid viral vector cargo comprises a polynucleotide encoding the rENV, and a transcription unit encoding a therapeutic protein.

In any aspect or embodiment described herein, the recombinant betaretrovirus ENV protein as described or claimed herein, comprises a surface subunit (SU) domain including a recombinant receptor binding domain (RBD) and a scaffold domain, and a betaretrovirus transmembrane (TM) domain, and the SU domain is covalently or non-covalently bound to the TM domain.

In any aspect or embodiment described herein, the recombinant betaretrovirus ENV protein as described or claimed herein, comprises an RBD that binds specifically to a surface receptor on a target cell and/or target tissue. In certain embodiments, the RBD is a portion of a protein known to interact with an extracellular receptor domain on a target cell or target tissue. For example, in certain embodiments, the RBD is a domain of a viral protein that interacts with a receptor on the surface of a target cell or target tissue, e.g., RBD can be the receptor biding domain of HIV, which interacts with CD4 and co-receptors on T cells.

In any aspect or embodiment described herein, the recombinant betaretrovirus ENV protein as described or claimed herein, comprises an RBD that is a receptor binding ligand (RBL), e.g., a peptide, polypeptide or protein ligand, that binds specifically to a receptor of interest. For example, the RBD can be the extracellular domain or portion thereof of a cell surface protein or peripheral membrane protein that interacts with or specifically binds to a receptor on the target cell or target tissue of interest. In other embodiments, the RBD is a receptor binding ligand (RBL), e.g., a peptide, polypeptide or protein that is a ligand of a receptor on the target cell or target tissue of interest. For example, the RBL can be a peptide, polypeptide, or protein, including an antibody or antigen binding portion or fragment thereof of an antibody or immunoglobulin, a chemokine, cytokine or the like. As would be understood by the skilled artisan, any number of protein-based ligand molecules could be utilized in order to target the described viral vector to the intended cellular or tissue target, which are contemplated and encompassed by the present disclosure.

In an additional aspect, the disclosure provides betaretrovirus-based vaccines. For example, vectored delivery of biomolecules to a specific cell or tissue type, e.g., nucleic acid-based vaccine.

As described herein, MBRACE is a novel method for customized engineering of viral vectors to use a pre-specified receptor of interest. Briefly, a viral vector is modified for delivery to a desired cell- or tissue-type by choosing an appropriate receptor as a biomarker, and then engineering a betaretrovirus entry protein to use that particular biomarker as an entry receptor instead of its native receptor. This provides a means for precise targeting of the appropriate cell or tissue for delivery of the contents of the viral particle. The starting structure for development can be an existing betaretrovirus entry protein, or an ERV-encoded betaretrovirus entry protein. The diversity of betaretroviral proteins available can also be exploited by pre-selecting those with any desirable properties relevant to the particular application as a starting point for customization.

Current gene therapy platforms either result in non-specific delivery, or require incorporation of multiple, different proteins into each viral particle for the cell-specific binding and entry functions. The latter results in loss of efficiency due to the need to get different proteins to incorporate into the same particle in appropriate ratios. The ideal system is a single viral protein that combines both the target cell receptor-specific binding and membrane fusion functions into a single protein. MBRACE is an improvement over current approaches by creating a single, customizable, viral protein for target cell-specific binding and entry.

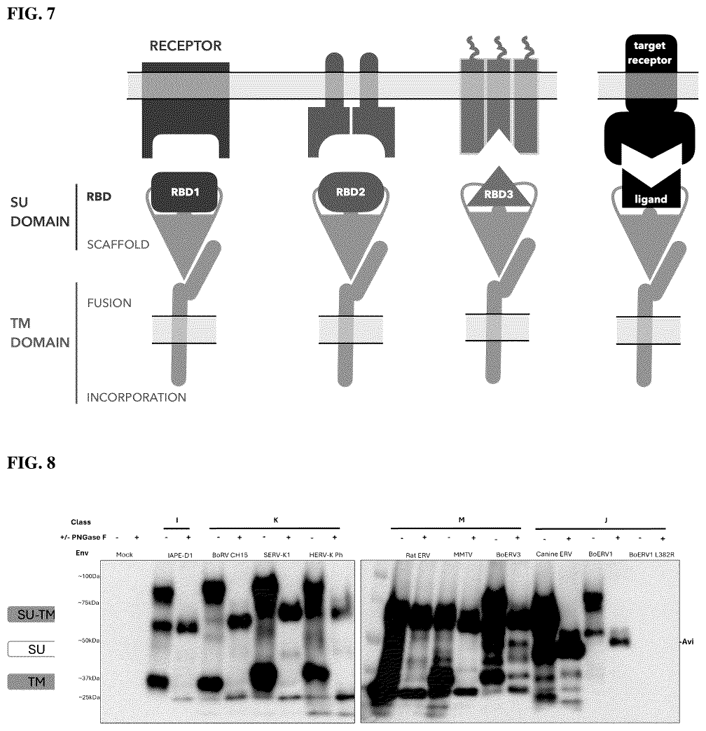

As described herein, developed herein is a surprising and unexpected framework region (FR) able to fold into a common “scaffold” that can link divergent receptor-binding domains with the highly conserved fusion apparatus provided by the TM subunit. The adaptability of the core betaretrovirus SU structure can be exploited by coupling a betaretrovirus “scaffold” with a ligand or ligand mimic for a heterologous receptor (FIG. 7). This approach can be used to “retarget” betaretrovirus ENVs to use pre-specified receptors for virus-mediated gene delivery.

While a conserved scaffold might have evolved to accommodate divergent variable region (VAR) domains (and receptor preferences), it is reasonable to assume that the scaffolds and VAR domains of the same viral lineage or structural class have acquired adaptations to work together optimally. This suggests that recombining heterologous ligands and scaffolds may result in suboptimal or nonfunctional ENVs. To address this issue, the large number of potential scaffolds is leveraged, in silico modeling is used to identify optimal scaffold-ligand combinations, and cell culture systems are used to iteratively test and refine combinations for optimal performance.

Sequences for nucleic acids containing betaretroviral element or recombinant betaretroviral envelope protein are shown in SEQ ID NO: 1-21, which can be utilized in the methods and compositions described herein.

African_Elephant_ERV-K19_Env Loxodonta africana
ERV-K19 NCBI RefSeq XM_023549157.1 LOC111750912,
transcript variant X2, mRNA
(SEQ ID NO: 1)
ACTCTACCTCTCATTCTTTTTTCGCCGAACTCTACCCCTCCTCTGGTAACCCTGTTCAT
TGCCGAGGCTGGCCCCCGGCAACTGGCGCCCGAACAGGGACCTGAAGGACGAAACTTCC
CCTTTGTGGATACCCGACAGACTCATCAGACATGCAAGACCCAAAAACCACCAAAATTC
CTCCAACACTTCCCCCCTCACTGCTCACAAACCTAACACCTCCCACGACTCTTCTGAGA
AATCTGTCTCTTGAAAATGCTCCATCTACACCCCCACCTTCGCCTGTCTCTAGTTCTCC
CTCCTCTTCATCTTCTACTCCACCCCTTCTCACTGACTTTATACAACCCTCTACAAACC
TCAACAACTCTTCATCTCGAGACAAAGTGCGGTGGAGAAGCCGGAGACATACTCCCTAT
TCAAGAACCCATCAAACCATTGCGGCTGAGCCCCCAACTTGGGGCCAACTCAAAAAACT
TACTCATCTGGCTCACATCTTAACCGTGGATCAACATATTCCCGTAAATGCGGGTACTC
TTTTTGTGGCAATGCTCGCTATCATTTCCTGTCAGGTATGTGTTGTTATGGCTGATATG
CATTGGGCTTATGTCCCAAAACCTCCTATATTTCACCCCGTCACCTGGGAAGATCCTTC
CCCCTCTGTATTTACTAATAATTCTGCATTACTGGGGGGCGAAACCATATTTTGGAACC
CTCAGGGCTTTAATTCTAATTATTCCTATTCTGGTATGTCAGATAATCCTCCTATTTGT
ATTCAATTAAGAAATTTGCGAAAAACTATCCCTGGCTGTATTCCTTTTGGCTTCAAATC
AATGATTACTGACTCACCTAATGGAAATCAAAACCAACAGCCTTACAGACTATTATGGG
CTCTACGCCTCATCCTTCCAGAAAAAACTGACTTTCTTAAAGGTATCCCAAAAGTACAC
ACTCCTCTTTTTCCTACCTGTGATAAGTCGCCTCCTGAGGATTCTAAGAGCAATCAAGA
TTGGGCTATGATTGACCCTTCAAAAGGATTTCCTATTTGGAGAAATTGTATGTATAATT
CACAAGTTGTATATGCTTTGCCTAAAATGTCTGCACGTCTTATTGATTGTAGTATTTTA
AATCCTGAAGATGATTATCGTCTATTTTTACAATCAGCTCGATATCGTTTTAATTGGCA
AGATACCTTAGTCCCTCTCCCACAAAAAGTTAATCGTTGGCATATAAATGGATGGGTTC
CCCCTATTCTTTCTACCTTCACGGGTAACATACATCCATCTTTGTGGAGACTGGCAACT
GCTGCCGGCAATGTCACTTTGTCCCGTCCTTTTAATAATGAGAGTTTTGATATTCAAGC
TTGTGTACCTGCTCCATATGTGTTATTAATTAGCCGCAACAATCACTCTAGTATTGTTA
TAGAAAACAAGAAAAAACATTATGTTAGTTCTTGTAAAAATTGTATACTGACCAGTTGT
ATAAATCCTGCAATTCCTGTTGAAAGTATGATGATTTTACAACAACCAAAATATATTAT
GATTCCTGTACATTTACAAGAAGACTGGTATGATGATTCAGGCCTAGAAGCCTTAAAAC
AAGCTTCTGCTATTTTATCTCGAGCCAAACGGTTTGTAGCAGCTTTAATACTGGGGATT
TCTGCTCTCATAGCTTTAGCAACTTCTCTTTCACTTTCTGCTATGGCGTTAACACAACA
GATTCATACTGCAAACTATGTAAATAATTTAAGTAAAAATATTACTTTAGCCTTAGCCA
CACAAGAAGCCATTGATAGAAAATTACAACAAGAAGTTAATGCCCTTGAAGAAGCAATT
CTATATATAGGACAAGAAATCACTAATCTCAAAGTGCGACTTACTTTGAGATGCCATTC
ATCTTCCGAATCTTAAATCAAGCTATCCAAAAGGTGGCTTCAGATCTTCATCTGGCGAA
ATTAAAAAATAAGAAAGGGGGAGATGCTGGGAGCCAGGCTGCAAGGGCGGCCCCTCTGC
ACCCAC
Black_Syrian_Hamster_(BSHRV)_ERV_env complete
genome Black Syrian Hamster strain BSHRV/2013/
HUN RV NCBI MK304634.1
(SEQ ID NO: 2)
ATGCTTGTGTGTTCCCACAGAATGAAAGCTCACCGATCTGGATTCCAGACAGACTTATC
AGACCCTACCAGCAGGAGACTCTCACGGAAAACAAAGAGGTGGAGAAACCGCGAGCTAC
AGACAATGGGGAAGCAGCTGACGAGTCTGACCATAACAAACAAAGAAGGTGAAGTCACC
TCAGAAGGTGAACTTAGCTGCAATCTGCCAACACAGACTCAACTCAAGAACCTACAAGG
AGAGGCTCTTACCGTACGGGCTCAAACAAAAACTCCTAAGACTCCTGTAGCAATTTTTC
TTGCTTTTCTTGCTATCTTATCAATGAATTCCGCGGTAAATGGAGAAAGTTACTGGGCA
TACATTCCCAAGCCTCCACTTTTACATCCGGTGGTTTGGGGAAATACAGAGCTCATTAA
AATTATGACTAATGTTACTGATGTGCTAGGGGGAACTCCTGATTTTCATATGGGAATTA
ATTCATCTGGGTATATAAATTATGAAGGTAGAGCAGATTCCTTACCAATCTGTATAGCA
CTTGATGGTATGCCTCCAGCTGGTTGTTTGCCTGTCAGCTATAGAACTTTCCTCACTGA
TACTCCTGATAATAAACCTGTTACAAGTAGGAGTCCAGAACAGCGGCATATGTGGCAAC
TACAAATTGCTACTTTGGGACACTCTAATTTTAATGAGACTATGGTGGTAAATCAGCCG
CTGCCTTCTAGATTGCCACATTGCATGGTAAAATACAAGAAAGATAGCTTATGGGAGGG
AGATGAAGATCAGATGCCTAATTGGCTTCATTGTGGATTTCCTCAAGCTGGAACAAGGT
TTTCTCCTTTGACTTCAGGAGAAATTATCTGGGATTTCTCTGTACCATCACCTGATCAT
GACTATTCTGGCTACCTTAAGGATCATTCTGCTCAATTTGGAGGATACACTCCTCCAAA
AAATGAGCCCATAAGGAGATGGTATTCAGCAGGGTGGGTGTCACCTGTATGGTATTTGC
AAAAACCGGATGGGACATATGGGACTCCTCAGCCAGATCTATTCAGGTTAGTTGCAGCA
GCCAATGTTATAATATTAAAGAAACCAAAGGCTGCCAAGTCAGAGCCTGTTCCTGCCAC
TGCTTGTCTCTCTTACCCATATGCCTTAATGTTTGGACGTAACAAATTGGTGCATATCA
AGAAGAAGGGGACTCATTTTTTTGTTAAATGTCGATCCTGCATATTAACTAATTGTGTA
CAATCAGGTTATGATAAATCCTTTTCAACTGTATTGATTGTTAAAAGGCCTGCTTATGT
GATGCTGCCAATAGATTTGGGTAATGATACTTGCTACGATAATTTGGCTATAAACTTGT
TTAACAAAATTAATGAATTGATTAGGCCAAAAGGATTTGTTGCATCATTAATTTTAGGA
ATCACAGCACTGATAGACATTTTGACTTCTTTTACAGTTTCTACAACAGCATTAGTAAA
GGAAATGCAGACAGCTTCCTTTGTAGATGACATGCATAGGAATGTCACCTTTGCCTTGT
CAGAACAGAGGTTAATAGATCTGAGATTAGAAACTAGATTAAATGCTTTAGAAGAGGCA
ATAATAGAAATGGGCCAAGACATACAAAATCTTAAGACACAATTAGCAACTAAATGCCA
TGCAGAGTTTAAGTATATCTGTGTTACACCTCTTCCATACAACTCTTCCTTTAATTGGA
ACAAGACTAAGGCACATTTATTAGGAGTATGGAAGGATACCAATTTAACCTATGATATC
AAGGCATTGCAGGAGAAAATTACAGTCATGAGTCAGAAAAAGATAGATACTTGGGACAT
TTCAGGCCTAGCATCAGAATTTTCAGAAAACCTAAGATCCCTGAATCCATTAAGCTGGA
CTCAGTATTTTGTTTTCCTAGGAATTGGAGCTGCTATTTTATTTACAGTGTGCTTGGTT
TTCCTATTCTTTTCAGAATACCACTTCACTCCATCGGCACCCTCCAGCGTGATTTCCAG
CTGTTCCTCTTAA
BoRV_CH15_env Bovine retrovirus beta-like
env NCBI accession KU720628
(SEQ ID NO: 3)
ATGGTCTTGTTCTTTTGCAGAATGGAAAACAACAATGGCTCCCGGCGTGTAGGATTCAA
GTACGGCGGACCACCGGCCAAGAAACAATTGCTACAGCCTCCGCGGTTTGACCCCACTG
TTACAGTTCCTGAAATAGAAATACCCTATCAGGCTTTTTGTCGTATGACGTTAGGAGAA
CGGCGCCGTAAACGGCCTGTGACGGTGCCTTTGCCTACATGGGGTCAGATAAAAAAGCT
TTCGCAAGAGGCCTTTCGCACTGTTATGGGAACGGGAAAAGTTCCTACACCTGAAAATC
TATTTCTTGCTATGTGTGCTATTTTGACTGTAACCTCCTCTCAGGCAGCTTCTCTCCCT
GTTCCTACTCCAGAAGCTGACGTAAGTTATACCTATTGGGCGTATATCCCAAATCCCCC
TCTGTTGGAAGCAACGGTTTGGGGAGTTTCAGATATTTTGATATATACTACTCCTCATG
TGTTGTCACCTCCTTGGAAGAATCTAACATATTTGAATAAAGATGATGGCCGCTTGTTT
AATTTTTCTCAGCGTTTAACCCAAGGAACTCCTATCTGTTTAACAAAGGAAGCTCGTGA
CCCCTGTTTGGTGATGGATTGGCAGAGTTGGCTTTTACCAGGGAGAAATGAAAGTTCAA
TTAGGGCACGTTTACAGCGATTGACGACTTGGTCAATTAATAATAGTTACAATAATATT
AGCTTGCAGGAAACTGCATCAGTGCCTTTTTGTACGCCCTTCACGTATATAAGTTTGAA
ATATAAACCATTTATTTGGTCTGAATGTACCAGTGAATGGGCCAAAAGAACTGGAGATT
ATATAAATTGGGGTCCCTTTGGTATGTTTTTTAATGATTGTTCGGAATGGAATAATAAA
ACTTGTCTTTTCTTTAACACTACCAGACTCCTGATGCCCATGAATCAAACACTTCAAGG
ATATAAATTGATGTGGTGGGGAGATGGTGGCGTCAGTAACCCGAGATGGACTCACGTGC
AGTATCCTGATAATTATCAATCTCATCTGTGGAAAGTGGCAGCGGCATTGCAACCAGTG
TACAAGGCTCAAGGGCAATTCCTCGGCACTTCTCGTTCCTTCTCAACTTATAGTTTCTT
TAATATCACCCAATATAATCTTACTGCTTGTATTCCATTACCTTATTTGTTTTTAGTAG
GGAATTTTATGTATAATGAAGGATTTATACAATGTAACAATTGTGCGTTATACTCATGT
CTTAATGCCTCCATTGATGCCCCTGCTATTATGATATTACAACAACGAACACATTTGTG
GTTGCCCGTGAAAATGGAACAGCCATGGGCATCTAGCCCCTTAGATAAAATAATAATAC
AAGTATTGAAGAAGGTTTTGCATCGTTCGAAAAGATTTATTGGAGCCGTAATTGCTACG
GTTCTGAGTTTGATTGCTATTGTTACCGTTGCTACCGTGTCTAGTGTGGCATTAAGCAC
AGCAATACAAAATAAACACTTTTTGGAAACATGGCATCAGGATTCTTTTCAATTTTGGA
CAGAACAAACTCAGATAGATGCACGATTTCAACAGCAGATAGATATATTAAAGCAGTCT
GTACAATGGTTAGGGAGAAAAATGATTCAATTAGAAAAGCAAATATCACTAAAATGCCA
CTGGAATAGTAGCACCTTTTGTATTACCCCTATATTGTATAACAAAACGGAATCAGACT
GGGTTGTTATACAACATGCCTTAGAAGGAAATATGACTGCGGTACAATTAATAGACGAT
TTGCAAAAAGAAGTAAATCAAGAATTTGGGAAACCTTATTCGAATGACTACTCAGGAGA
AATTGCTGACGATATATTTAAACATTTGGAGAAACTTAATCCTTTTACATGGGGACAAA
GTCTTATACATTCGGCTGGAAGTTTAGGAGTGTCTATTTTAATATTAATTATAAGTTTG
ATTTTGATATATCGGTGCTGCATAGTGCCTTTACAACTGTCTTTAACGCAAATGTGGAC
AAAAATGTTGATTTCTAAAAAGAAAAAAGGGGGAATTGAGGACATCGGCAAGAGTTAA
Bovine_ERV1_env NCBI XM_015463265.2 refseq
Bos taurus ERV-K113 Env polyprotein-like
(LOC107132283), mRNA
(SEQ ID NO: 4)
ATGCCGAAGCGCCACGCCGGCTGCTGGAAAGGTTGGTACGCACGCCGGAGACGATCTCT
AGCAAACCAGATGAGGAGACTGACGATTCAAGAGCATGATGAATTACCATCTCGGCAAC
AACTTCAGACATTGATGCGAGAGGCACAGAATAGTGTTGCTGTGCAGGGCGATCCGATA
TCGCCTTTAAGCTTCCTGATAACCTTATTATTTATCTTAAATCAGATTAATACTGCACA
TTCTGAAGAATATTCAAGTGCTTACTGGGCTTATTTTCCCAACCCTCCCCTTCTCCAAC
CGGTGGGCTGGGAGGGTCGGTCTATTCCCATATACACTAATGATACTGTGGCTTTGGGT
GGTTTTTCAGATAAACATATTATTCCTAATCAAGTTAATTTCTCTTATCATGGAGTGTT
TGATCGCTTACCTATTTGTCTGTCTAGATATTTTAATTCAACAGGATGTCTTTTTGTTG
GGGAGTCAGGATATGCTGATTATGACAATGGTTGTAGCCTGTATATTCCTGGAGTAATG
AAAGAGTCTGGAATTCCTCAACGTCGTAATGGAACTGGTCCTTTAGATATCCCTTTCTG
CGACTTTGGAACAAGGGGAAGAGTCACTGCTGCACCATGGAAACTGTGTCGAGATGGAA
TTGCTACGGTTTATAACATATTTGGGACAAATGAGTCATTGTGGGACTGGTCTCCAGAC
TCGGCCCGAAACCCTGGAGCTCAGGATAATAGGAAATTTGCATCCCGTATTTGGAACTT
GGGCGGCTCTAAAGTGTTCCAAACAGAAATCTGGAAACTAGCGGCCGCCCTTGGATGTG
AGGGTTCCTACCTTCAGGTGGGATCGAAAGTGAGACACGGTTATTTGTGGAATCTCACC
ATGAGATACCCTCGTGCATGTGTGCCTCACCCTTATGCTTTACTAGTAGGATCTGTAAA
TATACAACCTGGGTTACGAAATTATAATGTAACTTGTAACAATTGTACTTTGACTAACT
GTATTAGGGGAATTACTAATCAAACTAGGGTTCTAGTCTTAAGACAGCCTGCTTTTGTT
ATGGTTCCTGTAAAGATTAATGGGTCTTGGTATGATGAAAGGGGACTTGAACTTTGGAG
AGAAGTAGAGGGTGCTTTAATGCTCTATCGCAGGGGAATAGGGTTAATAATTCTGGGAT
TTGTAGCTTTGGTAACTCTTACTGCTTCCTGGATTACTGCAGCCCTGTCCTTGGCACAA
TCAGTGCAAACTGCAACTTTTGTTAATAATCTGGCACAAAATGCATCCGTTGCCCTGGG
GACTCAAGAAGATATTGACAAAAAGCTAGAGGATCGATTGAATGGCCTTTATGATGTTG
TAAAATTTTTAGGTGAAGAGGTTCAAAGTATAAAGTTGAGATTACGAGTACAATGTCAT
GCTGATTTTCAATGGATCTGTGTTACTCCCAAAAAATATAATGGTACTATAACTGCTTG
GGATAAGGTGAAAGCTCATTTAGAAGGTATTTGGCATAATGAAAATATTTCCTTAGATT
TGCTGCATCTACATCAGGAAATAATGAACATTGAAAATGCACCCAGACTTGATTTGGAT
GTTGCAAAAAGAGCTGAGTCTTTTGTTAAAGAACTTTTTCGACATGTACCTACTGTCAA
TAGTATTTGGTACTTGGGAGCAGCCATAGGAGGACTATTCTTAATAATTTTAACTTTTA
TTTCTTGCACCTTGTATAAT
Bovine_ERV2_env Bos taurus BERV-beta3_NCBI
EF030818.1
(SEQ ID NO: 5)
ATGACCCCTCAGGCTGAACAAACACTGAAGGTTACTGGAACTCCCATGACTCCAGATAA
GCTGTTTCTGGCAATGCTAGCTGTTCTCTCTTGCTCCTCTGGGGTAAGTGTGAAATATC
TTTACTGGGCATACCTACCTAATCCACCCTTGTTGTCCATACTGGATTGGGTCAACAAG
GCTCCTGTGGTATTCACCAATGACAGCCTGCACTTCCCAGCTCCATGGTCAACACAAAG
ACCAATTCATGAAGAAGATGAAGGAAGAAAAATAAATATTTCTATTGGATTCAAAACCT
TACCCATATGTTTCGGATTTCACCCTTTATGCATAACTATATTCCCCCAATGGTGGGCT
TACTCAGCACACAATGGTTCTGCCTATCGTATAGGAATGTTTGCAACCACATCCTTCCT
GCTAAATGGCTCTGAGAGGCATTATCCTGGACAAACAGTTAACTATATTAATTCACTCT
TCCAGCCTGAAGAACCAATATGTGATGTTGTATCATGGAGAAGGAAGCAATCAATTCAA
TCTCTATATTGGTCAGATTGTCAGGGACAGTTTGGGAAAATCTCTATCATTCAGCAGAA
TCACTTTATATTTAAATTCATTGACTGGGGGCCCCACAGATTGTTTGTGAACTGCTCTG
AGGAACAGGAAAAGAACCATAACTGTTCCTGGTATAATAAGTCCAATGTGTGGGGATTT
CATAAGACCAATACTAGCTTTTGGACTGATAAAGACATACTGTTAAATTGGTATAATGG
AGGATTGTCTCCACCTCGGCCCAGAATCATAGTCCCTACTGTGGGACCTGAGCAATGGC
GCGTGTGGAAGATTCCTGCTGCACTGGAGCACTTTGCCAGTGTGTATGGGACTGTGAAA
GCCTTAAAACCTCAAAACTATACCTTTTTATACAATAGTACAGGTTATATCCAATCTTG
TGTGCACCTCCCATACATGTTTATTGTGGGTAACTTTGATATGGATCTTTCTGCTAAAA
TTGTTAACAGTACAGCATGTGCCTTGTATACGTGTTTAAACCATACTATTTCATATCAT
AATGTCAACATTGCAGTAGTCAAGCAAAGATCTGAATTGTGGCTTCCTGTTAACCTAAC
AGAACCATGGACCGACTCTGTATTTCTCTCAGTTTTGCTCAAATATGGCTTAAGGAGGT
CAAAGTGCATAATAGGATGGATTATTGCTGGCATCCTTAGTCTAATATCTATTGTTACA
GTAGGAACCCTATCTGGTATGGCACTACACAATTCTATACAGAATCATGATTTTATAAC
TGCTTGGCACAAAGATTCCCATAATCTCTGGACTCAACAAGCTCAAATAGATCAGCAAT
TGCAAACACGAATTAATGAATTACAAACAGTGGTAATTAATATAGGAGATCAAGTACAG
CAACTTACTTTCCTAACACATATATGTTGTCACTGGAATTTTACTTCTTTCTGTCTGAC
TAACATGCCCTATAATGGCACTGAGTATCCTTGGGACAAAGTTAAAGTGCATTTTCAGG
ATCTCATGGATAACTCAAGTTTGGACATCAACCTTCTAAAACAACAAATTGCTAATTTT
CAAGTTAAAATACCTAAGGAATTATATAGCACCCAATTTTATGATATTTTAACCAAAAC
ATTATCCTCTCTGGATCCCAGAACTTGGTTTTCAGGAGGCAACTTTTTTATGTATGTAT
TAATTGCTCTGCTTTTTGTCATTGTTTGTAGGATACAGATGTCTCTACTCAAGACTCTG
GGCCTCCCACAGTGGAGTCCAACTAGCAGCTGCCATGCTTAAACTAAAAACAAAAGGAG
AATATGTTGGGAGGCACAGTGCAAAATAGCCTTGAAGGAGAAGGTCCTTGGGAAAATGG
CGAAGGGCCAAAAGTACCCAGGCATTGTGTACTGATAGCTGAAAAACATTTCCTGCCTT
TGGCTATGCTTGGTGCCAAGATTTAACAATTAAACATTAAAGACGTTGCTTGAGAAAAT
GGGTCATGGATAAACACATGGGTCGTTAAGAATGCGCTGGTCCTTGAAGTATCTTTTAC
TGATTATATCTTAGCCTTGTACGTGTTCTGCTGGCCATATGCTCTAAGCTCTTTGTTCC
TTGCTCCTATAAAAAGGCTTGACTGCAGAAATAAAGTTGTCAGTCCTTGCAGAGACTGT
CCAAGTGTTGTTCTTTCAGGTTCGCCGTTTCCAAGTCCCAGGGACATATCTCCCACA
Bovine_ERV3_(fematrin)_BERV-K1_env BERV-K1
(BoERV3 fematrin-1) provirus Baba, K et al
2011 NCBI: AB751366.1
(SEQ ID NO: 6)
ATGTCACAGCCCCCCAGGCATCTGGTCCCTCCACTGCCACGACCACAAAAAAGGAAGGG
ACCTCCTCTACCTCCACTCCCTCCGCCAACACGGGAAGCCCAGCTGATCTCTAGACGAA
TGACACCAGAGATCCTGGATGAACAGGAAGAGGAACCACCCACCAGACAGATGTCACAA
CTCTGTATAAGAGATATTGTTCTCCCTAATTCTTTACCCCACAGGAAATCACCGGGATG
TCCTACGCGGGCCACTCAACAAGCCTCTATACCTACCTGGGGACAGATTAAGACGCTTT
GTCACCAGGCACAAGGGATAGCTTCCCTGCAGGGCTCTCCAGTCTCTCCGGAGAGAGTG
TTTATTGCTATGCTTGCCTTACTATCTTGCCAGGTAAGCGCCTCCTCTCCCGCTCCAGA
AAAGTATTGGGCATACTTCCCAGATCCTCCAACCTTCCAAGTAGTTACTTGGAACAATG
CCCCTATACGGGTCCACACAAACCAGCCTCATTTATTGGGAGGACATTATACTTTCTAT
ACAGAGGAGGAATACCCTATTAATTTCAATTATACCTTTAGGGGATTAACAGATGATCT
CCCTGTTTGTTTTAACTTCCCTTTTGATCATACAGGAAGCTATATTACTCCTACCAAAG
AAGGATGTATTGGAGCCTCTGAAAAAGCAATTATAACAGATTCTCCATCCTTAAGATAT
CGGTCAGTTTGGGTATTACTGGCTCGTATGCCTGGGATCCTTGACCCCTATAAATCCCT
GCATTTTAAATCTCCACCAAAATATCCAAATTGTCATAATGCTGCCCCTTCAGATGCCA
TATGGAAAACTATAGATGGTCATTCAGGATATCCGATTTGGAAATCTTGTACTTATAAT
TCTAGAATTGACTACAGAATACCAGGAGGAAATTATACCATTCAGGACTGGAGCAACCC
GGATCCAGGTCAGGATCCAATGATTGATGATGCATTTAAAGGGAGATTTGACAATTGGA
AAGAATATTCAGTTCCTTGGCCATTACGCGCCACTAGATGGCATCGTAATCAATTTGTT
CCTCCTATGCTGTCTTATACGGCTAAGGGCAGAACTTATTGGCAACCAGAAATTTGGAA
GGCCCTTACCGCTACTGCCCCTATTACCTTAACCCGGCCAGAAAATATTTCTACTTATT
CTATTTTAGCTTGTCTACCCTCGCCTTATGTTTTTCTCTTCACTAATGATTCCAAGAGA
CTTAATATACACATGAATTATTCAGGTGGACCTAATATAGTAACTTGTGAACAATGTAT
GCTCTCATCTTGTTTGGCCCCTCAATATAATGTTTGCTCTTTTGTGGTGTTGCAACGCC
CACCCTATCTTATGGTGCCTGTAACTATAACTACACATTGGTATGATAACTATGGTCTT
GCTGTAATACAACAAGTGCGGGATTTAATGCGATCACGACGGTTAGCGGGCTTACTTTT
ACTGGGAATAGCAACTTTAATAACTGGAATTACTTCTGTTTATATGGCAGCAGTTTCAT
TGACTGAACAAGTACACACTGCTCAATATGTTGATGCTATGTCCAAAAATGTTTCTTTA
GCATTGGCAACAGAGGAAGCTATATTCAGGAAGCTGGAGATGAGGATAGATGCCCTAGA
GGAAGCAATATTGCATATTGGAAATGAATTGCAGGCTTTAAAAGTGAGATTGGCACTGT
CCTGTCATGCCGACTATCGGTGGATTTGTGTAACACCCCTGAAAGTAAATGAGACAGAT
TATGACTGGCAAAAAATTAAAAATCATATTTTAGGTGTCTGGAACAGCTCCGACATTAG
TTTAGATTTAAGGAAACTTCACAGTCAAATAACAACCATAGAACGCTCTCACTTAGATT
TTACTGCCTCAGGAGTAGCAAGTGACTTCTTCCATACAATCTCTAACTTTGTTTCAGGA
AAGTACTTTCTGTCTGCTATTTTTAACTATGCTGCTGTGGCCGCTATAATTCTGCTTAT
CATATTCATTCTTCCTTGTATTGTCAGAATTTTCCGACAGAGCATTCAGAAGCTCGAGA
CTGAATTGCATACGGGTTTCTTAAGAAATAAAAATGGGGGAGATGCCGGGACCCAGCAC
GGGAGTTCCCACCCATGA
Buffalo_ERV_env ref|XM_025267027.1|:1-2413
PREDICTED: Bubalus bubalis uncharacterized
LOC112579735 (LOC112579735), partial mRNA
(SEQ ID NO: 7)
TTAAAAATCCAAATTCAAAAATTAAAAGAAGGGGAGTTTAAGTATAGCTCTCCCCATCA
AATCTTGCAACATGCTCTTTTCGTAATAAATATTTTAAATGCCGATTCGGCCGGAATTA
CTGCAATGCTTCGCCATTGGTGCCCTGAGCAGCTGAATGCAAAACCATTAGTTAAATGG
AAGGACCTCCTTTCCGGACAATGGAAAGGTCCCGACCCATTGTTAACCAGTGGTCGAGG
ATATGCTTGCATTTTTCCACAGGACGCAGATTCTCCAATTTGGGTTCCAGACAGATTGA
TTCGCCATGTCTCAGCCCCTCGGATACCGGATTCCCCAGCTGCCGCGACCCCGAAAAAG
GAAGGGACCTCCTCTGCCTTCATTCCCCCCGCCAGCACGGGAAACGCCGCCGGCGTCGG
GACAACTGACATCGGAGATCCCGGATGAGCAAGGAACGGATCCACCCACCAAGCAGATG
TCTCAACTTCATATACAGGATATTGCTCCTCCCGATCCTTTGCCTCACAAAAGGGTGTC
AGGCCGCCTGACTCAGGCGACCCGGAATGCCTCCATACCTACTTGGGGACAAATAAAAA
CACTCTGCCACCAGGCACGGGGAATAGCTTCCTTACAGGGATCTCCAGCTTCTCCTGAG
AAACTGTTTATTGCCATGCTTGCTTTGCTTTCCTGTCAGGTAAGCGCCTCCCCTATTCC
AACCAAATATTGGGCGTATCTCCCAGATCCTCCAACTTTCCAAGTTGTTACTTGGGATA
ATGAGCCTATACGGGTCAACACAGACCAACCCCAGCTCTTGGGAGGATCCTATACTTCT
TACACTAGAGATAAATATCCTATTAACTTCAACTATACCTTCAGGGGATTAACAGGTGA
TTTCCCTGTTTGCTTTAACTTCCCCAATGACCTTAGAAGCTTTATTACCCCCACCAAAG
AAGGATGTGTTGGGGCCTCTAAAAAAATAATTATAACAGATTCCTGGTCTTTACGTCAT
CGATCAGCTTGGGTATTATTGGCTCGTATGCCCGGGATCCTTGACCCCTATGTAACCCT
GCATTCCAAATCTCCACCAAAATATCCGAATTGTTATAAGGTTGCTCCTTCAGAGAAAG
TATGGAACACCATAGACAGTCGTACAGGGTATCCGACTTGGACATCTTGTACTTATGAT
TCAAGGATTGATTATAGGATACCAGGAGGTGGAGATTATACTATTCAGGACTGGAGCAA
CCCGAATCCAGGTGAGGATCCATTGGCCAATGATACATTTGAGGGCAGATTCGAGAATT
GGGATAGGATTCCTCTTCCTTGGCCAACACATGCCACTAGAAGGCATCGTAATCAATTT
GTTCCTCCCATGCTGTCTTATACAACGAAGAAACAGACCTATTGGCAACCTGATATTTG
GAGAGCCCTTGCTGCTACTGCTCCTGTCTCCTTGTACCGCCCAGATAGCAATTCTACTT
ATTCTGTTTTAGCTTGTCTACCCTCCCCTTATGTTTTTCTTTTTACTAATGACTCCAAG
AAGCTTAATGTACGCATGAATTATTCAGGTGGACCTAATATAGTAACTTGTGAACAATG
TATGCTTTCATCTTGTTTGACCCCTCAATATAATGTTTGCTCTTTTGTGGTGTTACAAC
GCCCACCCTATCTCATGATACCTGTGACAGTGACTAGCCACTGGTATGACAATTACGGC
CTCGCCGTGTTACAACAACTACGAGATTTAATGCGACAACGGCGATTTGTGGGCTTACT
TATTTTAGGAATATCGGCTTTGATAGCAGCAATTACTTCTGTTACTGCGGCAGCAATAT
CATTGAGTCAACAAGTGCATACTGCCCAATATGTTGATACCATGTCTAAAAATGTCTCT
TTAACTCTAGCAACACAGGAAGTTATAGATAGAAAATTGGAGATGAGAGTAGATGCCTT
AGAAGAGGCAATAATGCATATTGGGACTGAGTTACAGGCGTTAAAAGTGAAAATGGCTC
TGTCTTGCCACGCTGATTACCGGTGGATATGCGTGACATCTTTAAAGGTAAATGAGACA
GATTATGAATGGGAAAAAATAAAAAATCATATCTCAGGTGTTTGGAACAGCTCTGACAT
TGGCCTGGACTTAGGGAAACTTCATAATCAAATACAGACCCTGGAACACTCTCGACTGG
ATTTTACTGCCGCTGGGGCAGCTAATGACTTTTTTCACACTTTCTCTGACTTCATTTCA
GGAAAAAACATTCTGTCTAATATCCTTGGATACATCGCTGCTGGTGCTTTAGTTTTGCT
TCTCATATTCATTCTTCCTTGTATTGTCAGGATTCTTCGGCAGAACATTCAGAAGCTCG
CGACTGAGCTGCATCTGGCTGTTTTAAGAAATAAAAAAGGGGGAGATGCTGGG
Canine_ERV-K6_env Canis lupus familiaris
(Boxer, Tasha) ERV-K6 env, mRNA
LOC106559572 NCBI XM_014118262.1
(SEQ ID NO: 8)
ATGAGGACACCCCGCGCTGGCTCCCGGAGTGATGGTGTCGACTCCTCACCCCTACGCCG
GAGACTAGCCAACCTAACCATAACTCGCCAACAAAAAAGAAGAAGGAGACGTCTCCCAT
CCTACTGTTGGGCCCGGATAAGGGAACTGATCAATCAAGCGATAAGTCTGGCTGAACAG
ACACAGCCTAATGAGTCCTTACTTACTATCCTTCTTACTGTATTAGCTATTACCACCCC
TCCCGTTGGTGAAGCGGCTGTATATTGGGCCTATCTACCCGACCCACCTACTATAAGGC
CGGTTACTTGGGATGATCCTACTCCCCAAATACACACCAATATGACAGAATATTTTGGA
GGGAGTAGTTCTGATGACATGGAGGTTGCAAACCATTCTATCAATTGGACGGGGCTGGC
AGCTCAGGTACCCATGTGCTTCCAATTACCACTATGTGACCAAAAACCAGTTAATGGTT
GCTTGGTAGTTCAGCCAGTACCTCACATAGTAACATATCTCTCAGAAATGGAAAAGAAT
GAAAACCCTCCATCCCGACTTAACATAATGTTAGAAAGGTGGATTGTCTATGAGGTCAC
TGGTGACACCCCTCCCAGTGACCCTCAACAAGGTAGGCGCCCTCCCGGGTACCCCAATT
GTCCTCAAAACTTAACCTATGCTATTTCAGGTCCTATTTGGACAGAGTGCCTTCAGAAG
ACACCTTTGGTCATCTCTCCTCACGGCATCCCTGAATTTTCTATCACAGACTGGTCGAG
AATGACAAGAAAGGAGCCTCTGCGCTATATTGCATATCCACCTGGTCGACCAATGCATA
ATGTGACTATACCCGGAGGAGTTTTTTCCACCGTCGGAGAAATGTACCACGCAGAACTC
TGGAAATTGGTCACCGCTATGGGACCAGCCAAGAGGCAGATGGCTAAGAGCGGCTACAC
GAACACAAAGGACTTCAATCTTACTATACAGGCCTGTGTCCCTAAGCCGTTCCTATTGG
TGGTAGGATATGGAACGACAAATTTTTCCTTTATGCAAAATAATGGAACTGATATGTTA
TTTGAACTAGGATGTAAAAATTGTGTATTAACCAATTGTTTGCCTGTGCACTTGCCCTC
ACCTCAAACAGGTCCTATCACTATCTTTTTGACTATGCAACCGCCTTATTTACTATTAC
CTGTTGAGATTGAAGGCCCTTGGTATGCTAATTATGGTTATCAGTTTGCTGTGGAATTG
CAGCTACAATTAAAAAGATTGAAACGCTTCCTTGGGCTGTTGATAGCCGGTATTGCTGC
ATTGGTTGCTCTGATAGCTACTGCAGCTGCAGCCTCTGTGGCCTTATCCCAGGGCATAC
AGACAGCACAATACGTTAACAACTTAGCAAAAAACGTCTCTTACGCGCTGTCTACTCAG
GAGCGTATAGATCAAAAAATTCTCAGTIGTCTTGATGGGCTTGAAGAAGAGAAATCAGG
AGTAAAGGGGATCTTGGAGAGTATTTTATCCAGCTCTTCCATGTCACAGCAAGACAACA
AGTTGAGAGATTCACACGGACTCTGA
Donkey_ERV-K19_env Equus asinus Donkey ERVK-19
env NCBI XM_014832344.2
(SEQ ID NO: 9)
CCAGTTTTTCACCGCCGCCGCCTCTCCGGCGGGAACCTGGACTCTGCCCAGGCTGGACC
CTGGCAAGTGGCGCCCGAACAGGGACCCAACGGGACGCTTCCTCTCCTTTGTGGATTCC
TGATCGTTTGATCCGGCATGTACAGCAGGCGGTGCCGGACCCTTCCTCCGTTGAGAAGA
ACGGACAACAGCGCCCTGACAGAGACTGATGAGGATCATCCTCCTGTGATCCAGATGCG
GTGGCTTTCGCTCCGTGACGCACCCTATCAGCGTCTTCCTCCTCGATCGCCGCCGCCTC
CATATCCGCGCTCTAAAGCCACTCGAGCTGCGTCAATTCCTACATGGGGTCAACTAAAA
CACCTTACCCAAGAGGCAGAACACTTAGTTCGGGTGCAGGGGGGTACAATTTCCCCCTC
TACTCTGTTTCTAGCTATGTTAGCCTTGATTGCCTGTCAGGCAGGAAAGACCTCTGCCT
CCCTTACCCAATATTGGGCTTATGTTCCTAAACCCCCTCTATTCCATCCAGTAACCTGG
AAGGAAGGACAGATTCCTGTCACGAATGACCATCCTGAGTTATTAGGAGGTCTGTCTAT
ATACCATGATAAAATTGACATTAACAAAGATTTTTCTTTTCAAGGTACCTCTAGCAGTC
CCCCTATATGCTTTAATGTACACCCTGTAGTTCGGATGCCGGCTCCACTGCCAGCAACT
AACATAGCAGGCTGTATTGGAACCTCTATTAAGGGGTTACTTACAGACTCCCCTAACGG
AATGTGGAGAGATTTATGGTCCCTGCAACTATTACTGCCTGGGGACATGGGCCCCAGGG
TTCTGAACTCTGCCATCCACCCTCCAGGATATCCTACTTGTCCTGGAAGGACAATATCC
CCAGAGAAATTGCCTACTAATAGTGACTGGACGTCGGTTGGCAAACGAGCTGGCTTCCC
AAAGTGGAGGGAGTGTATGTATTCATCGAAAATTGTGATAAAAATACCTTCTTTTCCTG
GTGAGATATTTGATTGGAGCTGTCAGGACCCTTCAAAAGATTACAGAAATTATTTACGT
GAAACTGGTAAATACCGGTGGTCAGATCGTTACATAGCTCCTGAACTGGCAGTGGATCA
TTGGCATATGAATGGCTGGGTTCCCCCCATATTGTCGGCTGACTCTGGTGAGGTCCAGC
CTCACTTATGGCAGGCTGTAGCCGCTCTACACAACATTATCCTAAATAGGCCAGTGGGG
TCCCAGTCCTATAAGGTTAAGGCATGTATTCCTGCCCCTTATGTTTTCCTGGCTGCGCC
CACTCACAGTCGTGCAATGACTATTCTACCCACCACGGACGGTTCCATATATAACATTA
CCTGTACCAATTGTATTCTGACTAATTGTTTAAATCCTGATATAGACGTGGAAACTGTT
ATGATATTACAACAACCCCCCTATCTTATGCTACCCGTTCATTTGTCTGAACCTTGGTA
TGATGATGTTGGATTGCTTACTTTAAAACAGGCCACTGATCTCTTACAGCGCCCCCGCC
GATTTATCACTGCTTTGATATTGGGTATCTCAGCCTTGATTACATTAGCAACCTCTCTC
ACTGTATCTGCTGTAGCGCTGAGTAAACAAATACATACTGCCTCTTATGTAGACGATCT
TTCTAAGAATACCACTCTTGCCCTAACTACCCAAGAAATAATAGACGAAAAACTGGAGG
CAAAGGTAAACGCTCTGGAAGAGGCAATATTACAGATAGGACAAGAACTTACAAATTTG
AAGGTTAGACTGGCCTTGCGATGTCATGTGTCCTATAGATGGATCTGTGTGACTCCTCT
TAAGGTAAATCAATCCCTACACTCCTGGGAGAAAATTAAAAATCATATTCAGGGAATAT
GGAACCATTCAGACTTTAGTTTAGACATTAGTAATTTACATAGACAAATTAATAATATT
AATCAGGCTGAAGAATCCTTTTCTGCTTCCAAAGTAGCCACCAACTTTTTCACCTCTCT
GACCTCTTTCGTAGGGGAGAAAAATTGGAGCTCCCTTATTGTTAATATTGCTGTCACCA
TTGGTGCTCTAATGATTTGTCTTTGCCTTTTACCAATCTTTCTCAAATTAATATTCAAC
TCTATAAGTCGGGTTTCGGCAGAATTACATCTGTTGGCCTTAAAAAGTAAAAAAGGGGG
AGATGCTGGGAGCCATGGCTACATCATTTGATCGGCTGTTTGACAGGGCCCAGCCGATT
AACGCTGAGGGGTGCAGGGTCGCCCCTTGGTGAAGAATAACCGGCCAGCCCCTCACATA
GTTCTGAAACCTGTGCTGCTTCTAATTGCCCTTCATTCCTTTTCCTTTCTTAACCTCCA
GTTTTCACTCCTTTGGGTGGCTGCTTGGGAGGCACTACCTCCCAGGAAAATTAGATATA
GTAAGAGAATGTGACCAC
HERV-K108_env_NCBI Accession: AF164614
(SEQ ID NO: 10)
ATGAACCCATCAGAGATGCAAAGAAAAGCACCTCCGCGGAGACGGAGACATCGCAATCG
AGCACCGTTGACTCACAAGATGAACAAAATGGTGACGTCAGAAGAACAGATGAAGTTGC
CATCCACCAAGAAGGCAGAGCCGCCAACTTGGGCACAACTAAAGAAGCTGACGCAGTTA
GCTACAAAATATCTAGAGAACACAAAGGTGACACAAACCCCAGAGAGTATGCTGCTTGC
AGCCTTGATGATTGTATCAATGGTGGTAAGTCTCCCTATGCCTGCAGGAGCAGCTGCAG
CTAACTATACCTACTGGGCCTATGTGCCTTTCCCGCCCTTAATTCGGGCAGTCACATGG
ATGGATAATCCTACAGAAGTATATGTTAATGATAGTGTATGGGTACCTGGCCCCATAGA
TGATCGCTGCCCTGCCAAACCTGAGGAAGAAGGGATGATGATAAATATTTCCATTGGGT
ATCATTATCCTCCTATTTGCCTAGGGAGAGCACCAGGATGTTTAATGCCTGCAGTCCAA
AATTGGTTGGTAGAAGTACCTACTGTCAGTCCCATCTGTAGATTCACTTATCACATGGT
AAGCGGGATGTCACTCAGGCCACGGGTAAATTATTTACAAGACTTTTCTTATCAAAGAT
CATTAAAATTTAGACCTAAAGGGAAACCTTGCCCCAAGGAAATTCCCAAAGAATCAAAA
AATACAGAAGTTTTAGTTTGGGAAGAATGTGTGGCCAATAGTGCGGTGATATTACAAAA
CAATGAATTCGGAACTATTATAGATTGGGCACCTCGAGGTCAATTCTACCACAATTGCT
CAGGACAAACTCAGTCGTGTCCAAGTGCACAAGTGAGTCCAGCTGTTGATAGCGACTTA
ACAGAAAGTTTAGACAAACATAAGCATAAAAAATTGCAGTCTTTCTACCCTTGGGAATG
GGGAGAAAAAGGAATCTCTACCCCAAGACCAAAAATAGTAAGTCCTGTTTCTGGTCCTG
AACATCCAGAATTATGGAGGCTTACTGTGGCCTCACACCACATTAGAATTTGGTCTGGA
AATCAAACTTTAGAAACAAGAGATCGTAAGCCATTTTATACTATTGACCTGAATTCCAG
TCTAACAGTTCCTTTACAAAGTTGCGTAAAGCCCCCTTATATGCTAGTTGTAGGAAATA
TAGTTATTAAACCAGACTCCCAGACTATAACCTGTGAAAATTGTAGATTGCTTACTTGC
ATTGATTCAACTTTTAATTGGCAACACCGTATTCTGCTGGTGAGAGCAAGAGAGGGCGT
GTGGATCCCTGTGTCCATGGACCGACCGTGGGAGGCCTCGCCATCCGTCCATATTTTGA
CTGAAGTATTAAAAGGTGTTTTAAATAGATCCAAAAGATTCATTTTTACTTTAATTGCA
GTGATTATGGGATTAATTGCAGTCACAGCTACGGCTGCTGTAGCAGGAGTTGCATTGCA
CTCTTCTGTTCAGTCAGTAAACTTTGTTAATGATTGGCAAAAAAATTCTACAAGATTGT
GGAATTCACAATCTAGTATTGATCAAAAATTGGCAAATCAAATTAATGATCTTAGACAA
ACTGTCATTTGGATGGGAGACAGACTCATGAGCTTAGAACATCGTTTCCAGTTACAATG
TGACTGGAATACGTCAGATTTTTGTATTACACCCCAAATTTATAATGAGTCTGAGCATC
ACTGGGACATGGTTAGACGCCATCTACAGGGAAGAGAAGATAATCTCACTTTAGACATT
TCCAAATTAAAAGAACAAATTTTCGAAGCATCAAAAGCCCATTTAAATTTGGTGCCAGG
AACTGAGGCAATTGCAGGAGTTGCTGATGGCCTCGCAAATCTTAACCCTGTCACTTGGG
TTAAGACCATTGGAAGTACTACGATTATAAATCTCATATTAATCCTTGTGTGCCTGTTT
TGTCTGTTGTTAGTCTGCAGGTGTACCCAACAGCTCCGAAGAGACAGCGACCATCGAGA
ACGGGCCATGATGACGATGGCGGTTTTGTCGAAAAGAAAAGGGGGAAATGTGGGGAAAA
GCAAGAGAGATCAGATTGTTACTGTGTCTGTGTAG
HERV-K113_env HERV-K113 complete genome
NCBI AY037928
(SEQ ID NO: 11)
ATGAACCCATCGGAGATGCAAAGAAAAGCACCTCCGCGGAGACGGAGACACCGCAATCG
AGCACCGTTGACTCACAAGATGAACAAAATGGTGACGTCAGAAGAACAGATGAAGTTGC
CATCCACCAAGAAGGCAGAGCCGCCGACTTGGGCACAACTAAAGAAGCTGACGCAGTTA
GCTACAAAATATCTAGAGAACACAAAGGTGACACAAACCCCAGAGAGTATGCTGCTTGC
AGCCTTGATGATTGTATCAATGGTGGTAAGTCTCCCTATGCCTGCAGGAGCAGCTGCAG
CTAACTATACCTACTGGGCCTATGTGCCTTTCCCGCCCTTAATTCGGGCAGTCACATGG
ATGGATAATCCTATAGAAATATATGTTAATGATAGTGTATGGGTACCTGGCCCCACAGA
TGATTGCTGCCCTGCCAAACCTGAGGAAGAAGGGATGATGATAAATATTTCCATTGGGT
ATCGTTATCCTCCTATTTGCCTAGGGAGAGCACCAGGATGTTTAATGCCTGCAGTCCAA
AATTGGTTGGTAGAAGTACCTACTGTCAGTCCCATCAGTAGATTCACTTATCACATGGT
AAGCGGGATGTCACTCAGGCCACGGGTAAATTATTTACAAGACTTTTCTTATCAAAGAT
CATTAAAATTTAGACCTAAAGGGAAACCTTGCCCCAAGGAAATTCCCAAAGAATCAAAA
AATACAGAAGTTTTAGTTTGGGAAGAATGTGTGGCCAATAGTGCGGTGATATTACAAAA
CAATGAATTCGGAACTCTTATAGATTGGGCACCTCGAGGTCAATTCTACCACAATTGCT
CAGGACAAACTCAGTCGTGTCCAAGTGCACAAGTGAGTCCAGCTGTTGATAGCGACTTA
ACAGAAAGTTTAGACAAACATAAGCATAAAAAATTGCAGTCTTTCTACCCTTGGGAATG
GGGAGAAAAAGGAATCTCTACCGCAAGACCAAAAATAATAAGTCCTGTTTCTGGTCCTG
AACATCCAGAATTATGGAGGCTTACTGTGGCCTCACACCACATTAGAATTTGGTCTGGA
AATCAAACTTTAGAAACAAGAGATCGTAAGCCATTTTATACTATCGACCTAAATTCCAG
TCTAACAGTTCCTTTACAAAGTTGCGTAAAGCCCCCTTATATGCTAGTTGTAGGAAATA
TAGTTATTAAACCAGACTCCCAGACTATAACCTGTGAAAATTGTAGATTGCTTACTTGC
ATTGATTCAACTTTTAATTGGCAACACCGTATTCTGCTGGTGAGAGCAAGAGAGGGCGT
GTGGATCCCTGTGTCCATGGACCGACCGTGGGAGGCCTCACCATCCGTCCATATTTTGA
CTGAAGTATTAAAAGGTGTTTTAAATAGATCCAAAAGATTCATTTTTACTTTAATTGCA
GTGATTATGGGATTAATTGCAGTCACAGCTACGGCTGCTGTAGCAGGAGTTGCATTGCA
CTCTTCTGTTCAGTCAGTAAACTTTGTTAATGATTGGCAAAATAATTCTACAAGATTGT
GGAATTCACAATCTAGTATTGATCAAAAATTGGCAAATCAAATTAATGATCTTAGACAA
ACTGTCATTTGGATGGGAGACAGGCTCATGAGCTTAGAACATCGTTTCCAGTTACAATG
TGACTGGAATACGTCAGATTTTTGTATTACACCCCAAATTTATAATGAGTCTGAGCATC
ACTGGGACATGGTTAGATGCCATCTACAGGGAAGAGAAGATAATCTCACTTTAGACATT
TCCAAATTAAAAGAACAAATTTTTGAAGCATCAAAAGCCCATTTAAATTTGGTGCCAGG
AACTGAGGCAATTGCAGGAGTTGCTGATGGCCTCGCAAATCTTAACACTGTCACTTGGG
TTAAGACCATTGGAAGTACTACAATTATAAATCTCATATTAATCCTTGTGTGCCTGTTT
TGTCTGTTGTTAGTCTACAGGTGTACCCAACAGCTCCGACGAGACAGCGACCATCGAGA
ACGGGCCATGATGACGATGGTGGTTTTGTCGAAAAGAAAAGGGGGAAATGTGGGGAAAA
GCAAGAGAGATCAGATTGTTACTGTGTCTGTGTAGAAAGAAGTAGACATAGGAGACTCC
ATTTTGTTATGTATTAAGAAAAATTCTTCTGCCTTGAGATTCTGTTAATCTATGACCTT
ACCCCCAACCCCGTGCTCTCTGAAACATGTGCTGTGTCAACTCAGAGTTGAATGGATTA
AGGGCGGTGCAGGATGTGCTTTGTTAAACAGATGCTTGAAGGCAGCATGCTCCTTAAGA
GTCATCACCACTCCCTAATCTCAAGTACCCAGGGACACAAAAACTGCAGAAGGCCGCAG
GGACCTCTGCCTAGGAAAGCCAGGTATTGTCCAAGGTTTCTCCCCATGTGATAGTCTGA
AATATGGCCTCGTGGGAAGGGAAAGACCTGACCGTCCCCCAGCCCGACACCCGTAAAGG
GTCTGTGCTGAGGAGGATTAGTATAAGAGGAAGGAATGCCTCTTGCAGTTGAGACAAGA
GGAAGGCATCTGTCTCCTCCCTGTCCCTGGGCAATGGAATGTCTCGGTATAAAACCCGA
TTGTATGCTCCATCTACTGAGATAGGGAAAAACCGCCTCAGGGCTGGAGGTGGGACCTG
CGGGCAGCAATACTGCTTTGTAAAGCATTGAGATGTTTATGTGTATGCATATCTAAAAG
CACAGCACTTAATCCTTTACATTGTCTATGATGCCAAGACCTTTGTTCACGTGTTTGTC
TGCTGACCCTCTCCCCACAATTGTCTTGTGACCCTGACACATCCCCCTCTTTGAGAAAC
ACCCACAGATGATCAATAAATACTAAGGGAACTCAGAGGCTGGCGGGATCCTCCATATG
CTGAACGCTGGTTCCCCGGTTCCCCTTATTTCTTTCTCTATACTTTGTCTCTGTGTCTT
TTTCTTTTCCAAATCTCTCGTCCCACCTTACGAGAAACACCCACAGGTGTGTAGGGGCA
ACCCACCCCTACA
HERV-K_Phoenix_env HERV-K Phoenix env
Dewannieux et al 2006
(SEQ ID NO: 12)
ATGAACCCATCCGAAATGCAGAGAAAAGCACCACCAAGAAGAAGAAGACACAGAAATAG
GGCACCACTGACACATAAAATGAACAAGATGGTGACCTCTGAGGAACAGATGAAGCTGC
CAAGTACCAAGAAAGCCGAGCCCCCTACATGGGCTCAGCTGAAGAAACTGACACAGCTG
GCCACTAAGTACCTGGAAAACACCAAAGTGACTCAGACCCCCGAGAGCATGCTGCTGGC
CGCTCTGATGATCGTGTCCATGGTGGTCTCTCTGCCAATGCCAGCTGGAGCAGCCGCTG
CAAATTACACCTATTGGGCTTACGTGCCATTCCCACCCCTGATCCGGGCAGTCACATGG
ATGGACAACCCCATTGAAGTGTATGTCAATGATAGTGTGTGGGTCCCTGGACCAATCGA
CGATAGATGCCCTGCCAAGCCAGAGGAAGAGGGAATGATGATCAACATTAGCATCGGCT
ACCGCTATCCTCCAATTTGCCTGGGGCGAGCACCTGGATGTCTGATGCCAGCCGTGCAG
AATTGGCTGGTGGAGGTCCCAACTGTGAGTCCCATCTCACGGTTTACCTATCACATGGT
GAGCGGAATGTCCCTGCGACCCCGGGTGAACTACCTGCAGGATTTCAGTTATCAGCGAT
CACTGAAGTTTCGGCCTAAGGGGAAACCCTGCCCTAAAGAAATCCCAAAGGAGTCAAAA
AACACAGAAGTGCTGGTCTGGGAAGAGTGTGTGGCTAATAGCGCAGTCATTCTGCAGAA
CAATGAGTTCGGCACTATCATTGATTGGGCCCCTAGGGGGCAGTTTTACCACAATTGCT
CCGGACAGACCCAGTCTTGTCCAAGTGCACAGGTGAGCCCAGCAGTCGACTCCGATCTG
ACAGAATCTCTGGACAAGCACAAACATAAGAAACTGCAGAGTTTCTATCCCTGGGAATG
GGGAGAGAAGGGCATCTCAACTCCACGCCCCAAAATCATTTCACCTGTGAGCGGACCCG
AACATCCTGAGCTGTGGCGACTGACCGTGGCAAGCCACCATATTAGAATCTGGTCCGGC
AACCAGACTCTGGAGACCAGAGACAGGAAGCCCTTCTACACAGTGGATCTGAATAGCTC
CCTGACTGTGCCTCTGCAGTCTTGCGTCAAGCCCCCTTATATGCTGGTGGTCGGCAACA
TTGTGATCAAACCTGACTCCCAGACAATCACTTGCGAAAACTGTCGACTGCTGACTTGT
ATCGATTCTACCTTTAATTGGCAGCACAGAATTCTGCTGGTGCGCGCACGAGAGGGAGT
GTGGATCCCAGTCTCCATGGACAGGCCCTGGGAAGCTTCCCCTTCTATTCATATCCTGA
CTGAGGTGCTGAAGGGCGTCCTGAACAGGTCTAAACGCTTCATCTTTACCCTGATTGCC
GTGATCATGGGCCTGATTGCAGTCACCGCTACAGCAGCTGTCGCAGGAGTGGCACTGCA
CTCTAGTGTCCAGAGCGTGAACTTTGTCAATGACTGGCAGAAGAACAGTACCAGGCTGT
GGAATTCACAGTCAAGCATTGACCAGAAACTGGCCAACCAGATCAATGATCTGCGCCAG
ACAGTGATTTGGATGGGCGATAGACTGATGAGCCTGGAGCATAGGTTCCAGCTGCAGTG
CGACTGGAACACAAGCGATTTTTGTATTACTCCCCAGATCTACAATGAATCCGAGCACC
ATTGGGACATGGTGCGGAGACACCTGCAGGGGCGGGAAGACAACCTGACCCTGGATATT
TCCAAGCTGAAAGAACAGATCTTCGAGGCTTCTAAGGCACATCTGAATCTGGTGCCTGG
AACAGAGGCAATCGCAGGAGTGGCAGATGGACTGGCTAACCTGAATCCAGTGACCTGGG
TCAAGACAATTGGATCCACCACAATCATTAACCTGATTCTGATCCTGGTGTGCCTGTTT
TGTCTGCTGCTGGTCTGCAGATGTACACAGCAGCTGAGGCGCGACTCTGATCACCGGGA
GAGAGCTATGATGACTATGGCAGTGCTGAGTAAGAGGAAAGGCGGGAATGTGGGCAAGT
CAAAACGCGACCAGATCGTGACCGTCAGCGTG
Horse_ERV_env Equus caballus ERV mRNA GeneID:
100146208 NCBI Refseq XM_001914834.2
(SEQ ID NO: 13)
ATGCGGCGGCTTTCGCTCCGTGACGCACCCTATCAGCGTCCACCTCCTCGATCACCGCC
GCCTCCACATCCGCGCTCTAGAGCCACTCGAGCTGTGCCTATTCCTACATGGGGTCAAC
TAAAACACCTTACCCAAGAGGCAGAACACTTAGTTCAGGTGCAGGGGGGTACAATCTCC
CCCTCTACTCTGTTTCTAGCTATGTTAGCCTTGATTGCCTGTCAGGCAGGAAAAACCTC
TGCCTCCCTTAACCAATATTGGGCTTATGTTCCTAAACCCCCTCTATTCCATCCAGTAA
CCTGGAAGGAAGGACAGATTCCTGTCACGAATAACCATCCTGAGTTATTAGGAGGTCTG
TCTATATACCATGATAAATTTGACATTAACAAAGATTTTTCTTTTCAAGGTACCTCTAG
CAGTCCCCCTATATGCTTTAATGTGCGCCCTGTAGGGCAGATGCCGGCTCCACTGCCAG
TAACTAACATACCAGGCTGTATTGGAACCTCTATTAAGGGGCTACTTACAGACTCCCCT
AACGGAATGTGGAGAAATTTATGGTCCCTGCAACTATTACTGCCTGGGGACATGGGCCC
CAGGGCTCTGAACTCTACCATCCACCCTCCAGGATATCCTACTTGTCCTGGAAGGACTA
CATCCCCAGAGAGATTGCCTACTAATAGTGACTGGATGTCGGTTGACAAACGAACGGGC
TTCCCAAAGTGGAGGGAGTGTATGTATTCATCGAAAACTGTGATAAAAATACCTTCTTT
TCCCGGTGAGATATTTGATTGGAGCTGTCAGGACCCTTCGAAAGATTACAGAAATTATT
TACGTGAAACTGGTAAATACCGGTGGTCAGATCGTTACATAGCTCCTGAACTGGCGGTG
GATCGTTGGCATATGAATGGCTGGGTTCCCCCCATATTGTCGGCAGACTCTGGTGAGGT
CCAGCCTCGCTTATGGCAGGCCGTAGCCGCTCTACACAACATTATCCTAAATAGGCCAG
TGGGGTCCCAGTCCTATAAGGTTAAGGCATGTATTCCTGCCCCTTATGTTTTCCTAGCT
GCGCCCACTCACAGTCGCGCAATGATTATTCTACCCACCACGGACGGTTCCATATATAA
CATTACCTGTACCAATTGTATTCTGACTAATTGTTTAAATCCTGATATAAACGTGGAAA
CTGTTATGATATTACAACAACCCCCCTATCTTATGTTACCCGTTCATTTGTCTGGACCT
TGGTATGATGATGTTGGATTGCTTACTTTGAAACAGGCTACTGATCTTTTACAGCGCCC
CCGCCGGTTTATCACGGCTTTAATATTGGGTATCTCAGCCTTGATTGCATTAGCAACCT
CTCTCACTGTATCTGCTGTAGCGCTGACTAAACAAATACATACTGCCTCTTATGTAGAC
GATCTTTCTAAGAATACCACTCTTGCCCTAACTACCCAAGAAATAATAGACGAAAAACT
GGAGGCAAAGGTAAATGCTCTGGAAGAGGCAATATTACAGATAGGACAAGAACTTACAA
ATTTGAAGGTTAGATTGGCCTTGCGTTGTCATGTGTCCTATAGATGGATCTGTGTGACT
CCTCTTAAGGTAAATCAATCTCTACACTCTTGGGAAAAAATTAAAAATCATATCCAGGG
AATATGGAACCATTCAGACTTTAGTCTAGACATTAGTGACTTACATAGACAAATTAATA
ATATTAATCAGGCTGAAGAATCCTTTTCTGCTTCTGAAGTGGCCACCAACTTTTTCACC
TCTCTGACCTCTTTTGTAGGGGAGAAAAATTGGAGCTCCTTTATTGTTAATATTGCTGT
CACCATTGGTGCTCTAATGATTTGTCTTTGCCTTTTTCCAGTCTTTCTCAAGTTAATAT
TCAACTCTATAAGTCGGGTTTCAGCAGAATTACATCTGTTGGCCTTAAAAAGTAAAAAA
GGGGGAGATGCTGGGAGCCGGAGAGTGAAATAA
IAPE-D1_env IAPE env complete provirus -
Ribet et al 2008 supplemental data 1
(SEQ ID NO: 14)
ATGACTCCCAGATGGATCCCTTGGAAACTACCTCTAATCCTCATGCTACCTCTGTGGAT
TTGGGTTCCTAGTATATTTGGAGAGTATAGATGGGCAATTCTGTCTGCTTTCCCTAAGC
CTATGCCTGTTCGTCATAATACAGCTGTTTTTCCCAAGTTTTTTACGACCAATAAAACT
TTAGGCCTGCCATACCTACCATTTGACCCTATCTGGGCACCATTGGGAGAAAAACGCTC
TTTAAGGGAACGAGGTTCTCTCTGTTTCCAAATTTATGAATTAGGAGGTTGTATCAGAC
TCACCAGCCAAGCTTTGGGAATGTTTTTTAAGTATCGAGGAGGCGTGGTGAAAATAACC
CAAGACACTTCCAACAGAGATATAACCCTTACAAATCGGACCTTCTGGCATGAGGCAAC
TTGGGTTAATGGTACATTTCTACCACCTAACTTTTCAGATAAAGAACGTCCCAATCAAC
CAAAAATGGCTCCTCATTGTAGCTTGGAAGATGAAGGGCTGATCCTGCCTTGGTCTGAT
TGTCAATCCTCTGTCACTCGTTGGGCAGATCAGAGTAAAACTTTTTCCTTTTCTCCCAA
CATGATAGATGACCCAGAGCAGAAATATGTTATGAAAAAGGGACTTTTCATACAGGACT
TTAGAATGCATCCTTTTCATAAATGGGTACTTTGTGGGATTAATGGTAGCTGTACAGAT
CTTAATCCCTTGGTTTTCCTCCAAGGAGGAGCTGCTGGAAAAGCGATTTTTAATGGTAT
TTCAAAATTTGCTCAGTTTCATCAAGTTCTGTTACCTGATAGAACCATTTATCAAAATT
CTACTACTGAAATCACTGGATTTAATAAGACATTGATAAAGCAGACTAATTATTTACCT
ACTCCAGTTTGTGTGTACACCCCTTTCTTGTTTATTCTTTCTAATGGTTCCTTTGAATC
CTGCACTAATGAAACCTGCTGGATGTCACAATGCTGGAGCCTTAAGTGGGCCAGCCGTG
CAATGCTTGCCAAGATTCCACGATGGGTTCCTGTCCCCGTGGAGACCCCTTCCACCATA
ACCTTACACAGGCAAAAGCGAGACTTTGGTATCACTGCAGCCGTTGTGGTGGCAATGGC
TGCCAGTGCGGCGGCAGCTACAGCTGCTGGAATTGCTATGGCAACATCAGTCCAGTCAA
GCACAACTGTCGAGCAGTTGTCCTCCTCCGTGGCGGAGGCGATCGATCAACATTCAGTG
CTCAGTGCCCAATTAAAAGGTGGGCTAATGATAGTGAACCAGCGCATTGACCTGGTGGA
GGAAAGACTGGAAATTCTGCTCCAATTGGCTCAGCTTGGCTGTGATAAGAAATCAGTAG
CCTTATGTATTACTAGTGTCCAATATGAAAATTGGACACATGCAGCTAATCTATCTAAA
GAATTGTCTTTGTTTCTTACAGGAAACTGGTCTGAAGGATTTGACGAAAAACTTGAGGC
GCTACGTACAGCAGTGATGACGATTAATTCTACGCGCGTGGACCCATCATTGATCGATG
GAATCAAAGGACTTTCTTCCTGGATGTCCTCTGCATTCTCACATTTTAAAGAGTGGGTG
GGAGTGGGCCTGTTTGGTGCAACCCTGTGTTGTGGACTAGTGTTTCTTCTATGGCTGGT
ATGCAAACTCAGATCTCAACAGAAACGTGACAAGGTGGTAATTGCTCAAGCACTTGCTG
CCATAGAGCAAGGCACCTCCCCTGAAATTTGGCTATCTATTCTCAAGAAT
JSRV_env_JSRV from pCMV2JS21
(SEQ ID NO: 15)
ATGCCGAAGCGCCGCGCTGGATTCCGGAAAGGCTGGTACGCGCGGCAGAGGAACTCCCT
GACGCATCAAATGCAACGCATGACGCTGAGCGAGCCCACGAGTGAGCTGCCCACCCAGA
GGCAAATTGAAGCGCTAATGCGCTACGCCTGGAATGAGGCACATGTACAACCTCCGGTG
ACACCTACTAACATCTTGATCATGTTATTATTATTGTTACAGCGGGTACAAAATGGGGC
AGCTGCGGCTTTTTGGGCGTACATTCCTGATCCGCCAATGATTCAATCCTTAGGATGGG
ATAGAGAAATAGTACCCGTATATGTTAATGATACGAGCCTTTTAGGAGGAAAATCAGAT
ATTCACATTTCCCCTCAGCAAGCAAATATCTCTTTTTATGGCCTTACCACTCAATATCC
CATGTGCTTTTCTTATCAATCGCAGCATCCTCATTGTATACAGGTATCAGCTGACATAT
CATATCCTCGAGTGACTATCTCAGGCATTGATGAAAAAACTGGGAAAAAATCATACGGG
AACGGATCTGGACCCCTCGACATTCCGTTTTGTGACAAGCATTTAAGCATTGGCATAGG
CATAGACACTCCTTGGACTTTATGTCGAGCCCGGGTCGCATCAGTATATAACATCAATA
ATGCCAATGCCACCTTTTTATGGGATTGGGCACCTGGAGGAACACCTGATTTTCCTGAA
TATCGAGGACAGCATCCGCCTATTTTCTCTGTAAATACCGCTCCAATATACCAAACGGA
ACTATGGAAACTTTTGGCTGCTTTTGGTCATGGCAATAGTTTATATTTACAGCCCAATA
TCAGTGGAAGCAAATATGGTGATGTAGGAGTTACAGGATTTTTATATCCTCGAGCTTGC
GTGCCGTATCCATTCATGTTGATACAAGGCCATATGGAAATAACACTGTCATTAAATAT
TTATCATTTGAATTGTTCTAACTGCATACTGACTAATTGTATTAGGGGAGTAGCCAAAG
GAGAACAGGTTATAATAGTAAAACAGCCTGCCTTTGTAATGCTGCCCGTTGAAATAGCT
GAAGCCTGGTATGATGAAACTGCTTTAGAATTATTACAACGCATTAATACGGCTCTCAG
CCGCCCTAAGAGAGGCCTGAGCCTGATTATTTTGGGTATAGTATCTTTAATCACCCTCA
TAGCTACAGCTGTTACGGCTTCCGTATCTTTAGCACAGTCTATTCAAGCTGCGCACACG
GTAGACTCCTTATCATATAATGTTACTAAAGTGATGGGGACCCAAGAAGATATTGATAA
AAAAATAGAAGATAGGCTATCAGCTCTATATGATGTAGTCAGAGTCTTAGGAGAGCAAG
TTCAGAGCATTAATTTTCGCATGAAAATCCAATGTCATGCTAACTATAAATGGATTTGT
GTTACAAAAAAGCCATACAATACTTCTGATTTTCCATGGGACAAAGTGAAGAAACATTT
GCAAGGAATTTGGTTCAATACTAATCTATCGTTAGACCTTTTACAACTGCATAATGAGA
TTCTTGATATTGAAAATTCGCCGAAGGCTACACTAAATATAGCCGATACTGTTGATAAT
TTCTTGCAAAATTTATTCTCTAATTTTCCTAGTCTCCATTCGCTGTGGAAAACCCTGAT
TGGTGTAGGAATACTTGTGTTTATTATAATTGTCGTAATCCTTATATTTCCTTGCCTTG
TTCGTGGCATGGTTCGCGATTTTCTAAAGATGAGAGTTGAAATGCTGCATATGAAATAT
AGAAATATGTTACAGCACCAACATCTTATGGAGCTTTTAAAAAATAAAGAGAGGGGAGA
TGCGGGGGACGACCCG
MMTV_env q61_Gift of Susan R. Ross;
genebank accession entry AF228552
(SEQ ID NO: 16)
ATGCCGAAACACCAATCTGGGTCCCCGATCGGTTCATCCGACCTTTTACTGAGCGGAAA
GAAGCAACGCCCACACCTGGCACTGCGGAGAAAACGCCGCCGCGAGATGAGAAAGATCA
ACAGAAAAGTCCGGAGGATGAATCTAGCCCCCATCAAAGAGAAGACGGCTTGGCAACAT
CTGCAGGCGTTAATCTTCGAAGCGGAGGAGGTTCTTAAAACCTCACAAACTCCCCAAAC
CTCTTTGACTTTATTTCTTGCTTTGTTGTCTGTCCTCGGCCCCCCGCCTGTGACCGGGG
AAAGTTATTGGGCTTACCTACCTAAACCACCTATTCTCCATCCCGTGGGATGGGGAAAT
ACAGACCCCATTAGAGTTCTGACCAATCAAACCATATATTTGGGTGGGTCGCCTGACTT
TCACGGGTTTAGAAACATGTCTGGCAATGTACATTTTGAGGGGAAGTCTGATACGCTCC
CCATTTGCTTTTCCTTCTCCTTTTCTACCCCCACAGGCTGCTTTCAAGTAGATAAGCAA
GTATTTCTTTCTGATACACCCACGGTTGATAATAATAAACCTGGGGGAAAGGGTGATAA
AAGGCGTATGTGGGAACTCTGGTTGACTACTTTGGGGAACTCAGGGGCCAATACAAAAC
TGGTCCCTATAAAGAAGAAGTTGCCCCCCAAATATCCTCACTGCCAGATCGCCTTTAAG
AAGGACGCCTTCTGGGAGGGAGACGAGTCTGCTCCTCCACGGTGGTTGCCTTGCGCCTT
CCCTGACCAGGGGGTGAGTTTTTCTCCAAAAGGGGCCCTTGGGTTACTTTGGGATTTCT
CCCTTCCCTCGCCTAGTGTAGATCAGTCAGATCAGATTAAAAGCAAAAAGGATCTATTT
GGAAATTATACTCCCCCTGTCAATAAAGAGGTTCATCGATGGTATGAAGCAGGATGGGT
AGAACCTACATGGTTCTGGGAAAATTCTCCTAAGGATCCCAATGATAGAGATTTTACTG
CTCTAGTTCCCCATACAGAATTGTTTCGCTTAGTTGCAGCCTCAAGATATCTTATTCTC
AAAAGGCCAGGATTTCAAGAACATGACATGATTCCTACATCTGCCTGTGTTACTTACCC
TCATGCCATATTATTAGGATTACCTCAGCTAATAGATATAGAGAAAAGAGGATCTACTT
TTCATATTTCCTGTTCTTCTTGTAGATTGACTAATTGTTTAGATTCTTCTGCCTACGAC
TATGCAGCGATCATAGTCAAGAGGCCGCCATACGTGCTGCTACCTGTAGATATTGGTGA
TGAACCATGGTTTGATGATTCTGCCATTCAAACCTTTAGGTATGCCACAGATTTAATTC
GAGCCAAGCGATTCGTCGCTGCCATTATTCTGGGCATATCTGCTTTAATTGCTATTATC
ACTTCCTTTGCTGTAGCTACTACTGCTTTAGTTAAGGAGATGCAAACTGCTACGTTTGT
TAATAATCTTCATAGGAATGTTACATTAGCTTTATCTGAACAAAGAATAATAGATTTAA
AATTAGAAGCTAGACTTAATGCTTTAGAAGAAGTAGTTTTAGAGTTGGGACAAGATGTG
GCAAACTTAAAGACCAGAATGTCCACCAGGTGTCATGCAAATTATGATTTTATCTGCGT
TACACCTTTACCATATAATGCTTCTGAGAGCTGGGAAAGAACCAAAGCTCATTTATTGG
GCATTTGGAATGACAATGAGATTTCATATAACATACAAGAATTAACCAACCTGATTAGT
GATATGAGCAAACAACATATTGACACAGTGGACCTCAGTGGCTTGGCTCAGTCCTTTGC
CAATGGAGTAAAGGCTTTAAATCCATTAGATTGGACACAATATTTCATTTTTATAGGTG
TTGGAGCCCTGCTTTTAGTCATAGTGCTTATGATTTTCCCCATTGTTTTCCAGTGCCTT
GCGAAGAGCCTTGACCAAGTGCAGTCAGATCTTAACGTGCTTCTTTTAAAAAAGAAAAA
AGGGGGAAATGCCGCGCCTGCAGCAGAAATGGTTGAACTCCCGAGAGTGTCCTACACC
Naked_Mole_Rat_ERV-K25_env NCBI
XM_013066611.2_PREDICTED:
Heterocephalus glaber endogenous retrovirus
group K member 25 Env polyprotein-like
(LOC101715120), mRNA
(SEQ ID NO: 17)
ATGAAGACAATGCGCGATGGTTACCAGAACGGCTCGTGCACCAGGCAGATGTCTCTTCA
GCAGATTCTCCTCCTCCTCATGGCGATGGTCACGATGACTAAGTGTGAGATGTTTTGGG
CCTATGTCCCTGACCCCCCTCTTTTGCATCATCCTGTCACCTGGGAGGATTCACCAGTT
TCAGTATATGTGAATAATACTGCAATAGCTGGCCCCCCCTCACTCGGTCATATAAAAGA
AGAGATAAAAGAAGGTTTTAATTATTCAGGGAAAGGATGGGGAATCCCTATATGTTTTT
CACAAAATGGTACCGTAAAGAGTTGTTTAAATTTTACATGGGATACTTTTAATGAAGGG
TCTCTGTATGGCAACAAGAGACACAATATTCGAATGCCATGGCCAAGTTGTGGAGGCTC
TCTAGAGATAGATGATCCTCCCCAAGACTACCCCCAATGTATGAATAAGGCACTCACCA
ATAACAGAATCATACCATGGGCCAGATGTAGAGTGGACCGGCCGGCCATCTATAACTTC
ACCGATCAACAAGAAGTGCTACAGGATTGGAGTGCCCAATTGGACAATTGTCAAGGTGG
GGCGAACACAGAACAGCAGTTGACTGTGGGACTTTGGCGAGTGCCTTCCGGCCCTTTTC
AGCCCATGATATGGAAACTTATGGTTGCTATGGGAGGGATAAAAAGGATTGGTGTGGGA
CCAGACTCTGGGTTGGAGGGAAATATCACAGCTTGTGTGCCCCAGCCCTACTTACTGTT
ATTGGGAGGTTTTGAGATTGATTATGAAAATGGAACTCGTCAATTTAATGTAACATGTG
ATAATTGCACATTGTCAAATTGTGTGAAATTCGGGTATCAAAATGTTTTAATAATTCAT
CAGCCCTCTTTTGTAATGTTGCCAGTTAATCTTTCAGAACCATGGTATGCAGAAAGAGG
ACTTCAAATATTGGAGAGTCTAAATGGTTTATTGAAAAGAGAAAAAAGGGTGGCCGGTA
TGATTGTAGCTGGAATTGTTACCTTTATAGCATTTTTGATTAGCTTATCTACTGCTGTG
GTTGCTTTATCCCAGTCTGTGCAGACAGCAGCTTTTGTAGATAATCTTTCGATGAATGT
ATCACAAGCCTTAGAGTTGCAAGAAGATATTGATATAAAAATGGAAGAGAAGTTGAATG
CTCTTTTTGACACGGTGATATATTTGGGACGCTCCCTTGAGGCTCTCAAACATAGAAAC
CATTTACAGTGCCATGGAGAATATCGTTGGATCTGTGTCACATCACTAGCTTATAATAT
GACAGATTATAACTGGCAGATGGTCCAGGACCATCTAAAAGGGGCATGGTCACATGGCA
ATGAAACCTCAGACCTATTAAAGCTCCATCAGCAGATATTGAGCATTCAACAAGCGGGA
AGAATAAGTCTGTCACCTGCACAGGTGGCACAGTCTCTTTATGATCATTTAAAAACATT
GGTGCCTTCCTCATTCTTTGCTAAACAGATAGTGAGAGGCCTTGGACTACTGGCTGTCG
GACTTCTGCTTATGTTTCTTTTTTTTGTCATATTCAGCCAGTATGTTCGATCCATGTTC
TTAACACTGGCTGTTCAATTACATTATCAAAAATTGATCACCTCTAAGAGGCCGGTTAG
TCAATGA
Opossum_ERV-K6_env_NCBI XM_056824278.1_PREDICTED:
Monodelphis domestica endogenous retrovirus group
K member 6 Env polyprotein-like (LOC130458467),
mRNA
(SEQ ID NO: 18)
ATGGAGAGATCTGGACTCCCAGTAAGCATCTCAAACCGTGGAAGGGACCGCTTCCAGAT
GCAAGGGGAGCAGAGGAACAGGCTCACCAGTCTAGCACCGCACCGGCTGCCCGAGACCA
GGACGAGAAGCAGACGAGCATCGCTGCTGTCTCCAGTCAGCTACCTGATGGTCAAGCGC
AGGGCACCGGGATCGAAGGCAACACTGGTGAATGCACGCACTCTGAACACTGACATTAC
ATTTACTCTGATGGCATTCACTATTTTGACTTTGCTGAACATCTGTCATGCTGAAGTCT
ACTGGTCCATCGTGCCCAAACCTCCTATTTTGAGAATGTTAACTTGGATGGACAAGCCC
CCTCTGGTATATGTTAACAACACGGACTGGCTACCACACCCTATTCTAGCCAAACGTGT
TCCTCTAGAGTCTGAAGGGGCTCTGTACAATTATTCAGGATCTACTGTGTATCCTCCAT
TTTGTATGTCTTTTAGAAATTCGTTTATTGGTAATTCTTTCTGTGTACCACGCAGACAT
CAAGCGTGGATTGTCAAAAACGCCACTGACAACAGTTATGTAGGGGTTGGCATGGGTGT
GATAGGAATGGGAGATGAGTTAGCACGTAAGACGTTCCAACCTAAACACCCTGCTAACA
AGTACCTATGTCCAAATCAGATAGGTACGGTACAAAAGGAGTTGCCATGGACAGATTGT
TCTGTCCGAGTTCCCAGAAAGGTTAAGATGCCGGCAACCACAGACTTTAACATTTACAA
TTGGGCACCCAGGTTACGTTGTGGTCGCTCTGAACAACTGACACGGTTAGACAAATACA
CTGACTATGAGAAATGGCACATGCCATGTCAGAATTTTCAGAAAATTGAGGACTTACTA
GAAGTAGACATGGCTGGTTCTAGACTTGCTATTGAAGCAGGTCCTATACAGAACACACA
GTGGAAGATAGTGGCTGCCACGCAGCCGATATACAAGTTTAAGGTCAAGGTGAAGAACT
CAGGACTTGATCTTGAGTATGACAGTCACATCAATGTCACAGCATGTGTTTTTGCTCCT
TATGTAATGCTAGTAGGTAACAACCTTCGAATTTTTAAAAATGACAAAGGTCTATATGA
GATCAACTGCACAAAATGCCGACTACATCAATGTCTTAGTCCTAAACACCAAGATTCAT
ATGTTGCTATCATGTATCATCCACCTTTAATGTGGGTACCTGTCAACTTGACAGAGACG
TGGGCATCTACGCCTACTGTGCACATTGTTCAAAAGGCATTTAACATGGTTATACATAG
GTCCAAGAGAATGGTCTTTGCACTTGTAGGAGCCGTAGTCTCAGTAATTGCCATGATCA
CATCACTAACCACGGCAACATTGGCATTAACTTCCTCTATCCAGAACCATGAGTTCATA
CAGGAAGTGGTGTCTAACTCTTCCAAATTATGGCACACTCAGCAGAGTATTGACTTAGC
TTACAGAGATGAGCTGGACAGTTTAAAAGATGCTGTGTTTTGGTTAGGTAAAGAGGTTG
AAGCTTTACACACAAGGATTACTTATCCCTGTCATTACAATCAAACGGGTTATTGTATT
ACTCCGTTGCCATATGATAATGTGTCATATGAATGGCAACTGGTCGTTCAGCACATCAA
GGGTGCTTGGGGAAGTCATAACAGCACCTTGGACATTATTAAGTTGCAACAGCAGATCA
ACAAATTAGACCAAATTGTATATGAGAATGAAGCTGCAAATATAGCTGCAAATTGGGAA
GCAACTTTATCTTCCCTAGATCCCCAAAATTGGTTTGGTAGAGCTAATTTAACCAGCAT
TGGTACTATCATTTTATTCATATTATTGGCTATTGGTTTGATTATACTTTGCAGCAGAA
GCTGTAAAAACAGAGCCTACATTCGAGGCATACTGCCAGAATTGGCACGGTCTTATCAC
ACTAGGCAATTGGTGCCTGAAAATGTTGAATTGAACACAACCGTTTTATGA
Rat_ERV-K21_env_n PREDICTED: Rattus norvegicus
uncharacterized LOC102557368 (LOC102557368),
mRNA
(SEQ ID NO: 19)
ATGCCGAATCACCAATTTGGGTCCCAGACAGACTCATCAGGCCAGCCAAAGGAAACGGA
CATCCGTCCCCCACGCCAAAAGAGGCCGAACCGCCGGATAATGCGGAACCTGGCCAAAA
AGATCAAGGCGACTCGCCTAGAGCCAAAAATAAAACCACGCCAGACGCCGACGTGGACG
CAACTTAAAACCCTGACAGCTCAGGCAAAGGAAGTATTGAAGGCTGCACATGCCCCGGA
AACGGCAACCTTGCTGTTTGTGGCTATGCTTGCAGTCATGAGTGCGCCCACCGCTTTAG
GGGAAAGTTGTTGGGCTTACTTACCAAAGCCACCCATATTACATCCTGTAGGGTGGGAG
GTGTCGGATCACATCAAGGTTTTAACCAATCAAACATTAGTGATAGGGGGATCGCCTGA
CTTTCACCTTCTAAAGAACAGCTCGGGTTATGTAGATTTTGAGGGAAAGTCTGATTCTC
TGCCCATTTGCTTCTCTTTCTCCCCTTCAGTGCCAGTGGGCTGTTTTCAAGTGGGGAAA
AGGGTGTTTAACACAGATACCCCCACCTTGGATAATACCAAACTAGATGGAAAGGGCAA
TGGGCGGCGTATGTGGGAATTGTGGATGACCACCGTGAGCGATAAAGAAGAAAAAAGAC
AACATTATACCACAGCAAAGAATCTTCCGCCGCGCTACCCCCATTACAGAACAACATTT
AAAAAGGACGCCCTTTGGGAGGGAGATGAGAATGCTTTCCCGCGCTGGCTTCCATGTGC
CTTCCCTGACAATGGGGTAAGATTCTCTCCCTTAGGCGCTGCTGGACTTCTCTGGGACT
TCTCCATGTCCTCACCTGATAAAGATCAGAACAATTATCTCTCCACCAACTCAAAACTT
TATGGGAACTACGTGCCTCCAGTTGGAAAACCGATCAAAAGATGGTATGATGCGGGGTG
GGTAGAACCAACATGGTTCTGGGAGCCACTCCACACGGACCTCCCTGATAGACAGAACA
AACCACTGATAGAACATCCAGAACTGTTCCGACTGGTTGCTTCTAGTGAGAAACTTTTG
CTGAAGAAACCAGGAGCTNGCGATTATGATGCTGTTTCAGTTTCTGCTTGTGTTACCTA
TCCCTATACAATATTAGTAGGATTGCCACAATTGATTAACATAGATAAGGTTGAATCTA
TTTTTAGTATTAAGTGTACATCATACAAGCTGACTAATTACATTGATTCCACTGTATCG
GGATCTGTTATATTGATTGTCAGGAGACCTCCTTATGTTTTGTTACCAGTAGACTTGGG
AGATGAGCCATGGTTTGATGATTCTGCCATCCAGACTATTAGGTATGCTACGGGTTTGA
TTAGGGCAAAAAGATTTGTAGCAGCCTTAATTCTAGGTATAACCGCTTTGATAGCTATT
GTTACTTCTTTTGCTGTTTCTACTACTGCTTTGGTTAAGGAAATGCAGACTGCCACTTT
TGTTAATGATCTCCATAAGAATGTTACCCTTACATTATCAGAACAGAAGATTATAGATT
TGAAATTAGAAACTAGATTAAATGCCCTAGAAGAGGTAGTATTAGAATTAGGACGAGAT
GTTACAAACATTAAGACTAGGATGGCCACACGCTGTCACGCTAATTATGATTTTATTTG
TGTGACTCCTTTACCTTATAATGCTACAGAAGAATGGGAAAGGACCAAAACACATTTGC
TAGGCATATGGGATGATGATAACATTTCTTATAATATTCAGGAATTGACGTCCTTAATC
ACAGAAATGAGTAAGCAACATGTAGATGCAGTGGACCTCTCTGGATTGACAAGATCCTT
TGCGGATGGAGTTAAATCATTAAATCCAATCGATTGGACACAATATTTTATTTTTATTG
GAGTTGGGATTTTACTACTTGTTGTTGTACTAATGATCTTCCCAATTGTATTCCGATGC
TTTACAGATTCCATTGCCCAGGTTCAAACAGATCTCAATTTGGTGCTTTTAAAAAATAA
AAAAGGAGGAACTGCGGTGCCTACAGCAGAGATAACCGAGCTCCGGGACAAACATGACG
CCTGA
SERV-K1_env Macaca mulatta Env NCBI KU363811.1
(SEQ ID NO: 20)
ATGAACCCATCGGAGATGCAAAGAAAAGCGCCTCCACGGAGACAGAAACACCGCAATCG
AGCACCATTGACTCGCATGATGAACCAAGTGATGATATCAGAAGAACAGATGAAGTCAC
CACGCACCAAGAAGGCGGAGCTGCCGACCTGGGCACAGTTAAAGAAGCTGACACCGTTA
GCTGGAAAAAGCCTAGCTAGCACAAAGGTGACACAAACCCCAGAAAAAATGCTGCTTAC
AGCTTTAATGATTGTATCAACGGTGGTAAGTCTCCCCATGCCTGCAGGAGCAGCTGCAG
CTAATTATACCTACTGGGCCTATGTGCCTTTCCCGCCCTTAATTCGGGCAGTTACATGG
ATGGATAATCCTATTGAAGTATATGTTAATAATAGTGTGTGGGTACCTGGTCCCACAGA
TGATCGTTGCCCTGCCAAACCGGAGGAAGAAGGAATGATGATAAATATTTCCATTGGGT
ATCGTTATCCTCCTATTTGCCTAGGGAGAGCACCAGGATGTTTAATGCCTGCTATTCAA
AATTGGTTGGTAGAAGTACCTACTGTCAGTCCCACCAGTAGATTTACTTATCACATGGT
AAGCGGAATGTCACTCAAACCACAGGTAAACTATTTACAAGACTTTTCTTATCAAAGAT
CATTAAAATTTAGGCCAAAAGGGAAACCTTGCCCCAAAGAGATTTCCAGAGAATCAAAA
GATTTAGTTTGGGAAGAATGTGTGGCCGATAGTGCAGTGATATTACAAAACAATACATT
CGGAACAGTTATAGATTGGGCACCTAGAGGTCAATTCTACCACAATTGCACAGGACAAA
CTCAATTCTGTCCCAGTGCACTAGTGAGTCCAACTGTTGACAGTGATTTAACGGAAAAT
TTAGATAAACATAAGCACAAAAAATTACAGTCTTTCTACCCTTGGATATGGGGAGAAAA
GGGAATATCTACTCCAAGACCAAAAATGATAAGTCCTGTTTTTGGTCCTGAACATCCAG
AATTATGGAGACTTACTGTGGCTTCATACCGCCTTAGAATTTGGTCTGGAAATCAAACT
ATAGAAACAAGAGATTATAAGCCATTTTACTCTATCAACCTAAATTCCAGTCTAACAGT
TCCTTTACAAAGTTGTGTAAAGCCCCCTTATATGTTAGTCATAGGAAATATAGTTATTA
AACCAGACTCCCAAACTATAACTTGTGAAAATTGCAGATTGTTTACTTGCATTGATTCA
ACTTTTGATTGGCAGCACCGTATTCTGCTGGTGAGAGCAAGAGAAGGCGTGTGGATCCC
TGTGTCCATGGACCGACCATGGGAGGCCTCACCATCCATCCATATTTTGACTGAAGTAT
TAAAAGGCGTTTTAAGTAGATCCAAAAGATTCATTTTTACTTTAATTGCAGTGATTATG
GGATTAATTGCAGTCACAGCTACAGCCACTGTGGCAGGAGTTGCATTGCACTCTTCTGT
TCAGACAGTAAGCTTTGTTGACAATTGGCAAAAGAATTCCACAAGGTTGTGGAATTCAC
AATCTGGTATCGATCAAAAATTGGCAAATCAAATTAATGATCTTAGACAAACCGTCATT
TGGATGGGAGATAGACTCATGAGCTTGGAACATCGTTTCCAGTTACAGTGTGACTGGAA
TACGTCAGATTTTTGTATTATACCCCAAGTTTATAATGAGTCTAAGCATCACTGGGACA
TGGTTAGACGCCATCTACAGGGAAGAGAAGATAATCTCACTTTAGACATTTCTAAATTA
AAAGAACAAATTTTTGAAGCCTCTCAAAGCCACTTAAATATTGTGCCTGGAGCTGAGGC
ATTAGATCAAGTGGCAAAAAATCTTTATGAATTAAACCCCACGACTTGGATTAAGTCTA
TTGGAAACTCTACTGCAATAAATTTTGGAATTATGTGTCTCTGTTTAATCGGCTTGTTT
TTAGTGTGCTGGACCAGTCGAAGAATCCTGCGTCAAAATCGAGAGAACGAACAAGCCTT
CATCGCCATGGCACATTTATATAGAGGAAAAGGGAGGGAGAACGTTGCGGGAAGTCAGG
GACCTTGA
Star_Nosed_Mole_Beta-like_ERV_env PREDICTED:
uncharacterized transcript mRNA LOC105853409
[Condylura cristata] NCBI XM_012722668.1 refseq
(SEQ ID NO: 21)
GATCATCTTCAAACCAAGCAGCGCTTCAATAAACTGTGATAGAAGAAGAATCCTGAGTT
CGTGTTTCATTCCGCGCTGGACGCGGGTCGCAACAGCTGGTGCCGAAACCCGGGAACGC
ATCAGTAGGAACTCCAGTGGGGGGCCGTATTAAGAAGATTCAGAACTTCACACAGGAAG
CGGTCGACGTCGGTAAGTATCTTCAGGATCGACCGGACCCGATTTGGCTCCCTACACGG
CTGACGAGAGAAGTGGCGAGAATCCCAGACGGGGACGATGCTGAAGGTCGCGAGAACAT
TCCTGCTCCTGCGGATGCTGATAATAGTCCCCTCAGCTAGCCTCGATAACTATTTCTGG
GCCATCACTCGTACGTGGCCCACTCCTTTTCCTGTGCATAACGAGTCCCTGGTTTTGCC
TCAATTTTATGCTACTTCTTGCACCTATGGCACCCCTTGCAACCCTAAGCCGACCAGTA
TCCAAGAAGAAAAATTTAATGCTTCTAGATTCAGCCTGGCCGGGACCTTGTGCTTCTCG
GTTGCTCTCAATGCCACTGACTGCATCCTGCTTTCCGCCACCAATCTCTCTGTATTCGC
TGACCCACTCTTGCATCTAGACCCTGGACTTTCCGCTTCAGTGCTTGTTTCAGCCCTGA
CACAGGCAGCCGCGGGCAAAACTCAAGGATCCGGTTCTGCAAATGGGACATCGGATGCT
GTCAATGTTACCACCTTTTTGGTCAACGTCTCTGCGATCACTCCCAACCTCATTTCCAC
CAGCGAAAACTGGACCTTCAACAGGTCCTACCCTTTAACAGCTTGCAGAAAGACGGTGT
TGACCTTTCCTCCCGAGTTTGCTGATTGCAGGGATACACGCAGGCCCGCCCAAAAGGAT
GGTTGGGCCCTCTGGCCCAGCCGATTCGGCCGCTGGAAGGAAAAACGATTGATCGCCGA
CCATCCGCTGTCGGCTTGGGTTCTTCAAAATGTCAAGGGTGCGACTTGTGACCTCTCCC
CCTTCATTCATCTGCAGCCCTCGACTATCCATTTGTCTAATGTTACGGGGTGTCTTAAC
ACGACTTCAAAAAAACTCACCTTGCACACACATCGGTTGAAGGATAATTTCACTTCTCC
TGGAGGGGTTGTTTGTGTCGACCCCCCTTTTCTTTTTCTGCTTTTTAATGCGTCTTCAG
AATCTTCTATTGACTGCACAACGACAAATTGCTTGTTATCCCAGTGTTGGCGAAAGACA
CATACATATGCTCTTAGTCTGCGCCTACCAACATTTGTTCCTTTGCCAGTGCAAGCTAA
CCCACAGGATTTCCCTGTCACCGCCTTGATCCGATCAAGGTGCGATTTTGGACTTACCG
CGGCTCTGGTGGCGGCCATAGCTGCGTCCATTGGTGCGGCTGCCGCCGCTGCGGTTGCG
ATGCAGACCTCTGTTCAAACGGTAGCCGTGGTTAACGAGGTCATCCAAAAGACTTCCTC
TTCGCTACAAACTCAAGAGCGCCTTAATCGTCATCTCACCTCTGGCATCTTACTTTTAA
ATAAGCGCATTGATTTAAAATCAATGGAAAGTAATGTTCTGACTTTATTTGATATTGTT
AGCCTTAGCTGTGTCGCTAAGACCCCACATATGTGTGTAACTCCCTATTATGCTTCATT
GAATGAGTCGCGCCGCTTGAGTTCCCTCCTCGCTGGTAATTGGACGAGGGAATTTGAAT
GCTTGCAAAATAACTTTACTGCACAAATTGTTGCTCTTAACATGACCAAGGCTGAAGTG
GTGACTGTGCGCCAGTTTACGGATTGGCTTTGGTCCTCTTTTTCCTTTTTCAAGGAGTG
GGTTGGTGTTGGCATGTTTGCTGCAGTTCTTGTGGCAGGCCTACTGCTACTAGTATTTC
TTTGGTTTCGAACCGTGCGTGCGCACCGGCGGGACAAGGCGGCCATCACTCGAGCATTG
ATCGCAATGGATGCTGGCCACTCTCCCCAGGCTTGGATTAGCCTCCTGCGTGAAGCCTG
ACTCCTTGCCCTTGCACCCGAGGGGCGTTCCGGCCCATTGCACTTGGAGGGATGGAGCC
AGTGCATGGCCGGTAGCTTGCGGGTCGCAACAATTCTGGTTATTTATAATGAGGTTTTT
TTTTCTAACTAGGGATGATAACTGTTTTGGTTTGGTTGCTTGTGCTTTAGGGAAGGGGG
CATTGGCCTTTATTGCTTTTGTTTTCTTTGCTTGTTTTAAAACTTGAAAGGATTATGGG
AATGTACATTTGGTCTTTGTTTATGTTCCTACTGTTTGTCCAAGCCACTTGTCTTTAGC
TTTGGTTATGGAGCAACTGCAGGTTTAAATATA

Sequences for amino acids containing betaretroviral element or recombinant betaretroviral envelope protein, which can be utilized in the compositions and methods described herein, are shown in SEQ ID NO: 22-42.

African_Elephant_ERV-K19_Env_Loxodonta africana ERV-K19 NCBI RefSeq
XM_023549157.1 LOC111750912, transcript variant X2, mRNA
(SEQ ID NO: 22)
MQDPKTTKIPPTLPPSLLTNLTPPTTLLRNLSLENAPSTPPPSPVSSSPSSSSSTPPLLTDFIQ
PSTNLNNSSSRDKVRWRSRRHTPYSRTHQTIAAEPPTWGQLKKLTHLAHILTVDQHIPV
NAGTLFVAMLAIISCQVCVVMADMHWAYVPKPPIFHPVTWEDPSPSVFINNSALLGGE
TIFWNPQGENSNYSYSGMSDNPPICIQLRNLRKTIPGCIPFGFKSMITDSPNGNQNQQPYR
LLWALRLILPEKTDFLKGIPKVHTPLFPTCDKSPPEDSKSNQDWAMIDPSKGFPIWRNCM
YNSQVVYALPKMSARLIDCSILNPEDDYRLFLQSARYRFNWQDTLVPLPQKVNRWHIN
GWVPPILSTFTGNIHPSLWRLATAAGNVTLSRPENNESFDIQACVPAPYVLLISRNNHSSI
VIENKKKHYVSSCKNCILTSCINPAIPVESMMILQQPKYIMIPVHLQEDWYDDSGLEALK
QASAILSRAKRFVAALILGISALIALATSLSLSAMALTQQIHTANYVNNLSKNITLALATQ
EAIDRKLQQEVNALEEAILYIGQEITNLKVRLTLRCHSSSES
Black_Syrian_Hamster_ERV_Env (BSHRV) Black Syrian Hamster ERV Env NCBI
QDK64675.1
(SEQ ID NO: 23)
MLVCSHRMKAHRSGFQTDLSDPTSRRLSRKTKRWRNRELQTMGKQLTSLTITNKEGEV
TSEGELSCNLPTQTQLKNLQGEALTVRAQTKTPKTPVAIFLAFLAILSMNSAVNGESYW
AYIPKPPLLHPVVWGNTELIKIMTNVTDVLGGTPDFHMGINSSGYINYEGRADSLPICIAL
DGMPPAGCLPVSYRTFLTDTPDNKPVTSRSPEQRHMWQLQIATLGHSNFNETMVVNQP
LPSRLPHCMVKYKKDSLWEGDEDQMPNWLHCGFPQAGTRESPLTSGEIIWDFSVPSPDH
DYSGYLKDHSAQFGGYTPPKNEPIRRWYSAGWVSPVWYLQKPDGTYGTPQPDLFRLVA
AANVIILKKPKAAKSEPVPATACLSYPYALMFGRNKLVHIKKKGTHFFVKCRSCILTNCV
QSGYDKSFSTVLIVKRPAYVMLPIDLGNDTCYDNLAINLFNKINELIRPKGFVASLILGIT
ALIDILTSFTVSTTALVKEMQTASFVDDMHRNVTFALSEQRLIDLRLETRLNALEEAIIEM
GQDIQNLKTQLATKCHAEFKYICVTPLPYNSSFNWNKTKAHLLGVWKDTNLTYDIKAL
QEKITVMSQKKIDTWDISGLASEFSENLRSLNPLSWTQYFVFLGIGAAILFTVCLVFLFFS
EYHFTPSAPSSVISSCSS
BoRV_CH15_Env Bos taurus Bovine retrovirus beta-like env NCBI accession
AMS37619
(SEQ ID NO: 24)
MVLFFCRMENNNGSRRVGFKYGGPPAKKQLLQPPRFDPTVTVPEIEIPYQAFCRMTLGE
RRRKRPVTVPLPTWGQIKKLSQEAFRTVMGTGKVPTPENLFLAMCAILTVTSSQAASLP
VPTPEADVSYTYWAYIPNPPLLEATVWGVSDILIYTTPHVLSPPWKNLTYLNKDDGRLF
NFSQRLTQGTPICLTKEARDPCLVMDWQSWLLPGRNESSIRARLQRLTTWSINNSYNNIS
LQETASVPFCTPFTYISLKYKPFIWSECTSEWAKRTGDYINWGPFGMFFNDCSEWNNKT
CLFFNTTRLLMPMNQTLQGYKLMWWGDGGVSNPRWTHVQYPDNYQSHLWKVAAAL
QPVYKAQGQFLGTSRSFSTYSFFNITQYNLTACIPLPYLFLVGNFMYNEGFIQCNNCALY
SCLNASIDAPAIMILQQRTHLWLPVKMEQPWASSPLDKIIIQVLKKVLHRSKRFIGAVIAT
VLSLIAIVTVATVSSVALSTAIQNKHFLETWHQDSFQFWTEQTQIDARFQQQIDILKQSVQ
WLGRKMIQLEKQISLKCHWNSSTFCITPILYNKTESDWVVIQHALEGNMTAVOLIDDLQ
KEVNQEFGKPYSNDYSGEIADDIFKHLEKLNPFTWGQSLIHSAGSLGVSILILIISLILIYRC
CIVPLQLSLTQMWTKMLISKKKKGGIEDIGKS
Bovine_ERV1_Env Bos taurus ERV1 Env ERV-K113-like NCBI XM_015463265.2
refseq (LOC107132283)
(SEQ ID NO: 25)
MFVFSHRMPKRHAGCWKGWYARRRRSLANQMRRLTIQEHDELPSRQQLQTLMREAQ
NSVAVQGDPISPLSFLITLLFILNQINTAHSEEYSSAYWAYFPNPPLLQPVGWEGRSIPIYT
NDTVALGGFSDKHIIPNQVNFSYHGVFDRLPICLSRYFNSTGCLFVGESGYADYDNGCSL
YIPGVMKESGIPQRRNGTGPLDIPFCDFGTRGRVTAAPWKLCRDGIATVYNIFGTNESLW
DWSPDSARNPGAQDNRKFASRIWNLGGSKVFQTEIWKLAAALGCEGSYLQVGSKVRH
GYLWNLTMRYPRACVPHPYALLVGSVNIQPGLRNYNVTCNNCTLTNCIRGITNQTRVL
VLRQPAFVMVPVKINGSWYDERGLELWREVEGALMLYRRGIGLIILGFVALVTLTASWI
TAALSLAQSVQTATFVNNLAQNASVALGTQEDIDKKLEDRLNGLYDVVKFLGEEVQSIK
LRLRVQCHADFQWICVTPKKYNGTITAWDKVKAHLEGIWHNENISLDLLHLHQEIMNIE
NAPRLDLDVAKRAESFVKELFRHVPTVNSIWYLGAAIGGLFLIILTFISCTLYN
Bovine_ERV2_Env Bos taurus ERV2 (BERV-beta3) beta-like NCBI accession
ABM73647.1
(SEQ ID NO: 26)
MTPQAEQTLKVTGTPMTPDKLFLAMLAVLSCSSGVSVKYLYWAYLPNPPLLSILDWVN
KAPVVFINDSLHFPAPWSTQRPIHEEDEGRKINISIGFKTLPICFGFHPLCITIFPQWWAYS
AHNGSAYRIGMFATTSFLLNGSERHYPGQTVNYINSLFQPEEPICDVVSWRRKQSIQSLY
WSDCQGQFGKISIIQQNHFIFKFIDWGPHRLFVNCSEEQEKNHNCSWYNKSNVWGFHKT
NTSFWTDKDILLNWYNGGLSPPRPRIIVPTVGPEQWRVWKIPAALEHFASVYGTVKALK
PQNYTFLYNSTGYIQSCVHLPYMFIVGNFDMDLSAKIVNSTACALYTCLNHTISYHNVNI
AVVKQRSELWLPVNLTEPWTDSVFLSVLLKYGLRRSKCIIGWIIAGILSLISIVTVGTLSG
MALHNSIQNHDFITAWHKDSHNLWTQQAQIDQQLQTRINELQTVVINIGDQVQQLTFLT
HICCHWNFTSFCLTNMPYNGTEYPWDKVKVHFQDLMDNSSLDINLLKQQIANFQVKIP
KELYSTQFYDILTKTLSSLDPRTWFSGGNFFMYVLIALLFVIVCRIQMSLLKTLGLPQWSP
TSSCHA
Bovine_ERV3_(BERV-K1_fematrin)_Env Bos taurus ERV3 Env (BERV-K1
fematrin) NCBI NP_001232880.2 from RefSeq NM_001245951.2
(SEQ ID NO: 27)
MSQPPRHLVPPLPRPQKRKGPPLPPLPPPTREAQLISRRMTPEILDEQEEEPPTRQMSQLCI
RDIVLPNSLPHRKSPGCPTRATQQASIPTWGQIKTLCHQAQGIASLOGSPVSPERVFIAML
ALLSCQVSASSPAPEKYWAYFPDPPTFQVVTWNNAPIRVHTNQPHLLGGHYTFYTEEEY
PINFNYTFRGLTDDLPVCFNFPFDHTGSYITPTKEGCIGASEKAIITDSPSLRYRSVWVLLA
RMPGILDPYKSLHFKSPPKYPNCHNAAPSDAIWKTIDGHSGYPIWKSCTYNSRIDYRIPG
GNYTIQDWSNPDPGQDPMIDDAFKGRFDNWKEYSVPWPLRATRWHRNQFVPPMLSYT
AKGRTYWQPEIWKALTATAPITLTRPENISTYSILACLPSPYVFLFINDSKRLNIHMNYSG
GPNIVTCEQCMLSSCLAPQYNVCSFVVLQRPPYLMVPVTITTHWYDNYGLAVIQQVRDL
MRSRRLAGLLLLGIATLITGITSVYMAAVSLTEQVHTAQYVDAMSKNVSLALATEEAIF
RKLEMRIDALEEAILHIGNELQALKVRLALSCHADYRWICVTPLKVNETDYDWQKIKNH
ILGVWNSSDISLDLRKLHSQITTIERSHLDFTASGVASDFFHTISNFVSGKYFLSAIFNYAA
VAAIILLIIFILPCIVRIFRQSIQKLETELHTGFLRNKNGGDAGTQHGSSHP
Buffalo_ERV_Env Bubalus bubalis water buffalo ERV NCBI XP_025122812.1
(SEQ ID NO: 28)
MSQPLGYRIPQLPRPRKRKGPPLPSFPPPARETPPASGQLTSEIPDEQGTDPPTKQMSQLHI
QDIAPPDPLPHKRVSGRLTQATRNASIPTWGQIKTLCHQARGIASLQGSPASPEKLFIAML
ALLSCQVSASPIPTKYWAYLPDPPTFQVVTWDNEPIRVNTDQPQLLGGSYTSYTRDKYPI
NFNYTFRGLTGDFPVCFNFPNDLRSFITPTKEGCVGASKKIIITDSWSLRHRSAWVLLAR
MPGILDPYVTLHSKSPPKYPNCYKVAPSEKVWNTIDSRTGYPTWTSCTYDSRIDYRIPGG
GDYTIQDWSNPNPGEDPLANDTFEGRFENWDRIPLPWPTHATRRHRNQFVPPMLSYTTK
KQTYWQPDIWRALAATAPVSLYRPDSNSTYSVLACLPSPYVFLFTNDSKKLNVRMNYS
GGPNIVTCEQCMLSSCLTPQYNVCSFVVLQRPPYLMIPVTVTSHWYDNYGLAVLQQLR
DLMRQRRFVGLLILGISALIAAITSVTAAAISLSQQVHTAQYVDTMSKNVSLTLATQEVI
DRKLEMRVDALEEAIMHIGTELQALKVKMALSCHADYRWICVTSLKVNETDYEWEKIK
NHISGVWNSSDIGLDLGKLHNQIQTLEHSRLDFTAAGAANDFFHTFSDFISGKNILSNILG
YIAAGALVLLLIFILPCIVRILRQNIQKLATELHLAVLRNKKGGDAG
Canine_ERV-K6_Env Canis lupus familiaris (Boxer, Tasha) ERV-K6 env, mRNA
LOC106559572 NCBI XP_013973737.1
(SEQ ID NO: 29)
MRTPRAGSRSDGVDSSPLRRRLANLTITRQQKRRRRRLPSYCWARIRELINQAISLAEQT
QPNESLLTILLTVLAITTPPVGEAAVYWAYLPDPPTIRPVTWDDPTPQIHTNMTEYFGGSS
SDDMEVANHSINWTGLAAQVPMCFQLPLCDQKPVNGCLVVQPVPHIVTYLSEMEKNEN
PPSRLNIMLERWIVYEVTGDTPPSDPQQGRRPPGYPNCPQNLTYAISGPIWTECLQKTPL
VISPHGIPEFSITDWSRMTRKEPLRYIAYPPGRPMHNVTIPGGVFSTVGEMYHAELWKLV
TAMGPAKRQMAKSGYTNTKDFNLTIQACVPKPFLLVVGYGTTNFSFMQNNGTDMLFEL
GCKNCVLTNCLPVHLPSPQTGPITIFLTMQPPYLLLPVEIEGPWYANYGYQFAVELQLQL
KRLKRFLGLLIAGIAALVALIATAAAASVALSQGIQTAQYVNNLAKNVSYALSTQERIDQ
KILSCLDGLEEEKSGVKGILESILSSSSMSQQDNKLRDSHGL
Donkey_ERV-K19_Env Equus asinus ERV-K19 beta-like NCBI XP_014687830.1
(SEQ ID NO: 30)
MYSRRCRTLPPLRRTDNSALTETDEDHPPVIQMRWLSLRDAPYQRLPPRSPPPPYPRSKA
TRAASIPTWGQLKHLTQEAEHLVRVQGGTISPSTLFLAMLALIACQAGKTSASLTQYWA
YVPKPPLFHPVTWKEGQIPVTNDHPELLGGLSIYHDKIDINKDFSFQGTSSSPPICFNVHP
VVRMPAPLPATNIAGCIGTSIKGLLTDSPNGMWRDLWSLQLLLPGDMGPRVLNSAIHPP
GYPTCPGRTISPEKLPTNSDWTSVGKRAGFPKWRECMYSSKIVIKIPSFPGEIFDWSCQDP
SKDYRNYLRETGKYRWSDRYIAPELAVDHWHMNGWVPPILSADSGEVQPHLWQAVAA
LHNIILNRPVGSQSYKVKACIPAPYVFLAAPTHSRAMTILPTTDGSIYNITCTNCILTNCLN
PDIDVETVMILQQPPYLMLPVHLSEPWYDDVGLLTLKQATDLLQRPRRFITALILGISALI
TLATSLTVSAVALSKQIHTASYVDDLSKNTTLALTTQEIIDEKLEAKVNALEEAILQIGQE
LTNLKVRLALRCHVSYRWICVTPLKVNQSLHSWEKIKNHIQGIWNHSDFSLDISNLHRQI
NNINQAEESFSASKVATNFFTSLTSFVGEKNWSSLIVNIAVTIGALMICLCLLPIFLKLIENS
ISRVSAELHLLALKSKKGGDAGSHGYII
HERV-K108_Env NCBI Accession: AAD51798
(SEQ ID NO: 31)
MNPSEMQRKAPPRRRRHRNRAPLTHKMNKMVTSEEQMKLPSTKKAEPPTWAQLKKLT
QLATKYLENTKVTQTPESMLLAALMIVSMVVSLPMPAGAAAANYTYWAYVPFPPLIRA
VTWMDNPTEVYVNDSVWVPGPIDDRCPAKPEEEGMMINISIGYHYPPICLGRAPGCLMP
AVQNWLVEVPTVSPICRFTYHMVSGMSLRPRVNYLQDFSYQRSLKFRPKGKPCPKEIPK
ESKNTEVLVWEECVANSAVILQNNEFGTIIDWAPRGQFYHNCSGQTQSCPSAQVSPAVD
SDLTESLDKHKHKKLQSFYPWEWGEKGISTPRPKIVSPVSGPEHPELWRLTVASHHIRIW
SGNQTLETRDRKPFYTIDLNSSLTVPLQSCVKPPYMLVVGNIVIKPDSQTITCENCRLLTC
IDSTFNWQHRILLVRAREGVWIPVSMDRPWEASPSVHILTEVLKGVLNRSKRFIFTLIAVI
MGLIAVTATAAVAGVALHSSVQSVNFVNDWQKNSTRLWNSQSSIDQKLANQINDLRQT
VIWMGDRLMSLEHRFQLQCDWNTSDFCITPQIYNESEHHWDMVRRHLQGREDNLTLDI
SKLKEQIFEASKAHLNLVPGTEAIAGVADGLANLNPVTWVKTIGSTTIINLILILVCLFCLL
LVCRCTQQLRRDSDHRERAMMTMAVLSKRKGGNVGKSKRDQIVTVSV
HERV-K113_Env HERV-K113 Env NCBI AAL18258
(SEQ ID NO: 32)
MNPSEMQRKAPPRRRRHRNRAPLTHKMNKMVTSEEQMKLPSTKKAEPPTWAQLKKLT
QLATKYLENTKVTQTPESMLLAALMIVSMVVSLPMPAGAAAANYTYWAYVPFPPLIRA
VTWMDNPIEIYVNDSVWVPGPTDDCCPAKPEEEGMMINISIGYRYPPICLGRAPGCLMP
AVQNWLVEVPTVSPISRFTYHMVSGMSLRPRVNYLQDFSYQRSLKFRPKGKPCPKEIPK
ESKNTEVLVWEECVANSAVILQNNEFGTLIDWAPRGQFYHNCSGQTQSCPSAQVSPAV
DSDLTESLDKHKHKKLQSFYPWEWGEKGISTARPKIISPVSGPEHPELWRLTVASHHIRI
WSGNQTLETRDRKPFYTIDLNSSLTVPLQSCVKPPYMLVVGNIVIKPDSQTITCENCRLLT
CIDSTFNWQHRILLVRAREGVWIPVSMDRPWEASPSVHILTEVLKGVLNRSKRFIFTLIA
VIMGLIAVTATAAVAGVALHSSVQSVNFVNDWQNNSTRLWNSQSSIDQKLANQINDLR
QTVIWMGDRLMSLEHRFQLQCDWNTSDFCITPQIYNESEHHWDMVRCHLQGREDNLTL
DISKLKEQIFEASKAHLNLVPGTEAIAGVADGLANLNTVTWVKTIGSTTIINLILILVCLFC
LLLVYRCTQQLRRDSDHRERAMMTMVVLSKRKGGNVGKSKRDQIVTVSV
HERV-K_Phoenix_Env HERV-K Phoenix Env extracted from pCRV1-HERV-K
(SEQ ID NO: 33)
MNPSEMQRKAPPRRRRHRNRAPLTHKMNKMVTSEEQMKLPSTKKAEPPTWAQLKKLT
QLATKYLENTKVTQTPESMLLAALMIVSMVVSLPMPAGAAAANYTYWAYVPFPPLIRA
VTWMDNPIEVYVNDSVWVPGPIDDRCPAKPEEEGMMINISIGYRYPPICLGRAPGCLMP
AVQNWLVEVPTVSPISRFTYHMVSGMSLRPRVNYLQDFSYQRSLKFRPKGKPCPKEIPK
ESKNTEVLVWEECVANSAVILQNNEFGTIIDWAPRGQFYHNCSGQTQSCPSAQVSPAVD
SDLTESLDKHKHKKLQSFYPWEWGEKGISTPRPKIISPVSGPEHPELWRLTVASHHIRIWS
GNQTLETRDRKPFYTVDLNSSLTVPLQSCVKPPYMLVVGNIVIKPDSQTITCENCRLLTCI
DSTFNWQHRILLVRAREGVWIPVSMDRPWEASPSIHILTEVLKGVLNRSKRFIFTLIAVIM
GLIAVTATAAVAGVALHSSVQSVNFVNDWQKNSTRLWNSQSSIDQKLANQINDLRQTV
IWMGDRLMSLEHRFQLQCDWNTSDFCITPQIYNESEHHWDMVRRHLQGREDNLTLDIS
KLKEQIFEASKAHLNLVPGTEAIAGVADGLANLNPVTWVKTIGSTTIINLILILVCLFCLLL
VCRCTQQLRRDSDHRERAMMTMAVLSKRKGGNVGKSKRDQIVTVSV
Horse_ERV_Env_Equus caballus ERV mRNA NCBI Refseq XM_001914834.2
(SEQ ID NO: 34)
MRRLSLRDAPYQRPPPRSPPPPHPRSRATRAVPIPTWGQLKHLTQEAEHLVQVQGGTISP
STLFLAMLALIACQAGKTSASLNQYWAYVPKPPLFHPVTWKEGQIPVTNNHPELLGGLS
IYHDKFDINKDFSFQGTSSSPPICFNVRPVGQMPAPLPVTNIPGCIGTSIKGLLTDSPNGM
WRNLWSLQLLLPGDMGPRALNSTIHPPGYPTCPGRTTSPERLPTNSDWMSVDKRTGFPK
WRECMYSSKTVIKIPSFPGEIFDWSCQDPSKDYRNYLRETGKYRWSDRYIAPELAVDRW
HMNGWVPPILSADSGEVQPRLWQAVAALHNIILNRPVGSQSYKVKACIPAPYVFLAAPT
HSRAMIILPTTDGSIYNITCTNCILTNCLNPDINVETVMILQQPPYLMLPVHLSGPWYDDV
GLLTLKQATDLLQRPRRFITALILGISALIALATSLTVSAVALTKQIHTASYVDDLSKNTT
LALTTQEIIDEKLEAKVNALEEAILQIGQELTNLKVRLALRCHVSYRWICVTPLKVNQSL
HSWEKIKNHIQGIWNHSDFSLDISDLHRQINNINQAEESFSASEVATNFFTSLTSFVGEKN
WSSFIVNIAVTIGALMICLCLFPVFLKLIFNSISRVSAELHLLALKSKKGGDAGSRRVK*
IAPE-D1_Env IAPE env complete provirus-Ribet et al 2008 supplemental data 1
(SEQ ID NO: 35)
MTPRWIPWKLPLILMLPLWIWVPSIFGEYRWAILSAFPKPMPVRHNTAVFPKFFTTNKTL
GLPYLPFDPIWAPLGEKRSLRERGSLCFQIYELGGCIRLTSQALGMFFKYRGGVVKITQD
TSNRDITLTNRTFWHEATWVNGTFLPPNFSDKERPNQPKMAPHCSLEDEGLILPWSDCQ
SSVTRWADQSKTFSFSPNMIDDPEQKYVMKKGLFIQDFRMHPFHKWVLCGINGSCTDL
NPLVFLQGGAAGKAIFNGISKFAQFHQVLLPDRTIYQNSTTEITGFNKTLIKQTNYLPTPV
CVYTPFLFILSNGSFESCTNETCWMSQCWSLKWASRAMLAKIPRWVPVPVETPSTITLHR
QKRDFGITAAVVVAMAASAAAATAAGIAMATSVQSSTTVEQLSSSVAEAIDQHSVLSA
QLKGGLMIVNQRIDLVEERLEILLQLAQLGCDKKSVALCITSVQYENWTHAANLSKELS
LFLTGNWSEGFDEKLEALRTAVMTINSTRVDPSLIDGIKGLSSWMSSAFSHFKEWVGVG
LFGATLCCGLVFLLWLVCKLRSQQKRDKVVIAQALAAIEQGTSPEIWLSILKN
JSRV_Env JSRV Env extracted from full length pCMV2JS21
(SEQ ID NO: 36)
MPKRRAGFRKGWYARQRNSLTHQMQRMTLSEPTSELPTQRQIEALMRYAWNEAHVQP
PVTPTNILIMLLLLLQRVQNGAAAAFWAYIPDPPMIQSLGWDREIVPVYVNDTSLLGGKS
DIHISPQQANISFYGLTTQYPMCFSYQSQHPHCIQVSADISYPRVTISGIDEKTGKKSYGN
GSGPLDIPFCDKHLSIGIGIDTPWTLCRARVASVYNINNANATFLWDWAPGGTPDFPEYR
GQHPPIFSVNTAPIYQTELWKLLAAFGHGNSLYLQPNISGSKYGDVGVTGFLYPRACVP
YPFMLIQGHMEITLSLNIYHLNCSNCILTNCIRGVAKGEQVIIVKQPAFVMLPVEIAEAWY
DETALELLQRINTALSRPKRGLSLIILGIVSLITLIATAVTASVSLAQSIQAAHTVDSLSYNV
TKVMGTQEDIDKKIEDRLSALYDVVRVLGEQVQSINFRMKIQCHANYKWICVTKKPYN
TSDFPWDKVKKHLQGIWFNTNLSLDLLQLHNEILDIENSPKATLNIADTVDNFLQNLFSN
FPSLHSLWKTLIGVGILVFIIIVVILIFPCLVRGMVRDFLKMRVEMLHMKYRNMLQHQHL
MELLKNKERGDAGDDP
MMTV_Env MMTV Env from q61 plasmind; genebank accession entry AF228552
(SEQ ID NO: 37)
MPKHQSGSPIGSSDLLLSGKKQRPHLALRRKRRREMRKINRKVRRMNLAPIKEKTAWQ
HLQALIFEAEEVLKTSQTPQTSLTLFLALLSVLGPPPVTGESYWAYLPKPPILHPVGWGN
TDPIRVLTNQTIYLGGSPDFHGFRNMSGNVHFEGKSDTLPICFSFSFSTPTGCFQVDKQVF
LSDTPTVDNNKPGGKGDKRRMWELWLTTLGNSGANTKLVPIKKKLPPKYPHCQIAFKK
DAFWEGDESAPPRWLPCAFPDQGVSFSPKGALGLLWDFSLPSPSVDQSDQIKSKKDLFG
NYTPPVNKEVHRWYEAGWVEPTWFWENSPKDPNDRDFTALVPHTELFRLVAASRYLIL
KRPGFQEHDMIPTSACVTYPHAILLGLPQLIDIEKRGSTFHISCSSCRLTNCLDSSAYDYA
AIIVKRPPYVLLPVDIGDEPWFDDSAIQTFRYATDLIRAKRFVAAIILGISALIAIITSFAVAT
TALVKEMQTATFVNNLHRNVTLALSEQRIIDLKLEARLNALEEVVLELGQDVANLKTR
MSTRCHANYDFICVTPLPYNASESWERTKAHLLGIWNDNEISYNIQELTNLISDMSKQHI
DTVDLSGLAQSFANGVKALNPLDWTQYFIFIGVGALLLVIVLMIFPIVFQCLAKSLDQVQ
SDLNVLLLKKKKGGNAAPAAEMVELPRVSYT
Naked_Mole_Rat_ERV-K25_Env Heterocephalus glaber ERV-K25 beta-like Env
NCBI XP_012922065.1 from Refseq XM_013066611.2 LOC101715120 mRNA
(SEQ ID NO: 38)
MKTMRDGYQNGSCTRQMSLQQILLLLMAMVTMTKCEMFWAYVPDPPLLHHPVTWED
SPVSVYVNNTAIAGPPSLGHIKEEIKEGFNYSGKGWGIPICFSQNGTVKSCLNFTWDTFN
EGSLYGNKRHNIRMPWPSCGGSLEIDDPPQDYPQCMNKALTNNRIIPWARCRVDRPAIY
NFTDQQEVLQDWSAQLDNCQGGANTEQQLTVGLWRVPSGPFQPMIWKLMVAMGGIK
RIGVGPDSGLEGNITACVPQPYLLLLGGFEIDYENGTRQFNVTCDNCTLSNCVKFGYQN
VLIIHQPSFVMLPVNLSEPWYAERGLQILESLNGLLKREKRVAGMIVAGIVTFIAFLISLST
AVVALSQSVQTAAFVDNLSMNVSQALELQEDIDIKMEEKLNALFDTVIYLGRSLEALKH
RNHLQCHGEYRWICVTSLAYNMTDYNWQMVQDHLKGAWSHGNETSDLLKLHQQILSI
QQAGRISLSPAQVAQSLYDHLKTLVPSSFFAKQIVRGLGLLAVGLLLMFLFFVIFSQYVR
SMELTLAVQLHYQKLITSKRPVSQ
Opossum_ERV-K6_Env_Monodelphis domestica ERV-K6 NCBI accession
XP_056680256.1 from RefSeq XM_056824278.1 LOC130458467 mRNA
(SEQ ID NO: 39)
MERSGLPVSISNRGRDRFQMQGEQRNRLTSLAPHRLPETRTRSRRASLLSPVSYLMVKR
RAPGSKATLVNARTLNTDITFTLMAFTILTLLNICHAEVYWSIVPKPPILRMLTWMDKPP
LVYVNNTDWLPHPILAKRVPLESEGALYNYSGSTVYPPFCMSFRNSFIGNSFCVPRRHQA
WIVKNATDNSYVGVGMGVIGMGDELARKTFQPKHPANKYLCPNQIGTVQKELPWTDC
SVRVPRKVKMPATTDFNIYNWAPRLRCGRSEQLTRLDKYTDYEKWHMPCQNFQKIEDL
LEVDMAGSRLAIEAGPIQNTQWKIVAATQPIYKFKVKVKNSGLDLEYDSHINVTACVFA
PYVMLVGNNLRIFKNDKGLYEINCTKCRLHQCLSPKHQDSYVAIMYHPPLMWVPVNLT
ETWASTPTVHIVQKAFNMVIHRSKRMVFALVGAVVSVIAMITSLTTATLALTSSIQNHEF
IQEVVSNSSKLWHTQQSIDLAYRDELDSLKDAVFWLGKEVEALHTRITYPCHYNQTGYC
ITPLPYDNVSYEWQLVVQHIKGAWGSHNSTLDIIKLQQQINKLDQIVYENEAANIAANW
EATLSSLDPQNWFGRANLTSIGTIILFILLAIGLIILCSRSCKNRAYIRGILPELARSYHTRQL
VPENVELNTTVL
Rat_ERV-K21_Env_PREDICTED: Rattus norvegicus uncharacterized
LOC102557368, mRNA NCBI XM_039097077
(SEQ ID NO: 40)
MPNHQFGSQTDSSGQPKETDIRPPRQKRPNRRIMRNLAKKIKATRLEPKIKPRQTPTWTQ
LKTLTAQAKEVLKAAHAPETATLLFVAMLAVMSAPTALGESCWAYLPKPPILHPVGWE
VSDHIKVLTNQTLVIGGSPDFHLLKNSSGYVDFEGKSDSLPICFSFSPSVPVGCFQVGKRV
FNTDTPTLDNTKLDGKGNGRRMWELWMTTVSDKEEKRQHYTTAKNLPPRYPHYRTTF
KKDALWEGDENAFPRWLPCAFPDNGVRFSPLGAAGLLWDFSMSSPDKDQNNYLSTNS
KLYGNYVPPVGKPIKRWYDAGWVEPTWFWEPLHTDLPDRQNKPLIEHPELFRLVASSE
KLLLKKPGAADYDAVSVSACVTYPYTILVGLPQLINIDKVESIFSIKCTSYKLTNYIDSTV
SGSVILIVRRPPYVLLPVDLGDEPWFDDSAIQTIRYATGLIRAKRFVAALILGITALIAIVTS
FAVSTTALVKEMQTATFVNDLHKNVTLTLSEQKIIDLKLETRLNALEEVVLELGRDVTNI
KTRMATRCHANYDFICVTPLPYNATEEWERTKTHLLGIWDDDNISYNIQELTSLITEMSK
QHVDAVDLSGLTRSFADGVKSLNPIDWTQYFIFIGVGILLLVVVLMIFPIVFRCFTDSIAQ
VQTDLNLVLLKNKKGGTAVPTAEITELRDKHDA*
SERV-K1_Env Macaca mulatta Env NCBI AMA68119.1
(SEQ ID NO: 41)
MNPSEMQRKAPPRRQKHRNRAPLTRMMNQVMISEEQMKSPRTKKAELPTWAQLKKLT
PLAGKSLASTKVTQTPEKMLLTALMIVSTVVSLPMPAGAAAANYTYWAYVPFPPLIRAV
TWMDNPIEVYVNNSVWVPGPTDDRCPAKPEEEGMMINISIGYRYPPICLGRAPGCLMPA
IQNWLVEVPTVSPTSRFTYHMVSGMSLKPQVNYLQDFSYQRSLKFRPKGKPCPKEISRES
KDLVWEECVADSAVILQNNTFGTVIDWAPRGQFYHNCTGQTQFCPSALVSPTVDSDLTE
NLDKHKHKKLQSFYPWIWGEKGISTPRPKMISPVFGPEHPELWRLTVASYRLRIWSGNQ
TIETRDYKPFYSINLNSSLTVPLQSCVKPPYMLVIGNIVIKPDSQTITCENCRLFTCIDSTFD
WQHRILLVRAREGVWIPVSMDRPWEASPSIHILTEVLKGVLSRSKRFIFTLIAVIMGLIAV
TATATVAGVALHSSVQTVSFVDNWQKNSTRLWNSQSGIDQKLANQINDLRQTVIWMG
DRLMSLEHRFQLQCDWNTSDFCIIPQVYNESKHHWDMVRRHLQGREDNLTLDISKLKE
QIFEASQSHLNIVPGAEALDQVAKNLYELNPTTWIKSIGNSTAINFGIMCLCLIGLFLVCW
TSRRILRQNRENEQAFIAMAHLYRGKGRENVAGSQGP
Star_Nosed_Mole_Beta-like_ERV_Env PREDICTED: uncharacterized protein
LOC105853409 [Condylura cristata] NCBI XP_012578122.1
(SEQ ID NO: 42)
MLKVARTFLLLRMLIIVPSASLDNYFWAITRTWPTPFPVHNESLVLPQFYATSCTYGTPC
NPKPTSIQEEKFNASRFSLAGTLCFSVALNATDCILLSATNLSVFADPLLHLDPGLSASVL
VSALTQAAAGKTQGSGSANGTSDAVNVTTFLVNVSAITPNLISTSENWTFNRSYPLTAC
RKTVLTFPPEFADCRDTRRPAQKDGWALWPSRFGRWKEKRLIADHPLSAWVLQNVKG
ATCDLSPFIHLQPSTIHLSNVTGCLNTTSKKLTLHTHRLKDNFTSPGGVVCVDPPFLFLLF
NASSESSIDCTTTNCLLSQCWRKTHTYALSLRLPTFVPLPVQANPQDFPVTALIRSRCDFG
LTAALVAAIAASIGAAAAAAVAMQTSVQTVAVVNEVIQKTSSSLQTQERLNRHLTSGIL
LLNKRIDLKSMESNVLTLFDIVSLSCVAKTPHMCVTPYYASLNESRRLSSLLAGNWTREF
ECLQNNFTAQIVALNMTKAEVVTVRQFTDWLWSSFSFFKEWVGVGMFAAVLVAGLLL
LVFLWFRTVRAHRRDKAAITRALIAMDAGHSPQAWISLLREA

### MBRACE Pipeline

MBRACE is based on re-targeting an MBRACE scaffold ENV to bind to user-specific receptor(s). Customization begins by choosing a suitable MBRACE ENV scaffold and modifying its aminoacid sequence to contain an inserted, in-frame peptide ligand that binds the desired cell-surface receptor. In-frame ligand sequences can include (but are not limited to) natural ligands of the receptor, including cellular ligands or heterologous receptor binding domains from other viruses; peptides derived by in vitro evolution, screening or selection for binding to the receptor (e.g. by phage-display); and specialized binding proteins, such as DARPins or ScFVs, that are specific for the receptor. Once a candidate ligand is identified, an MBRACE ENV scaffold sequence is modified to include the ligand sequence inserted in-frame at predetermined positions corresponding to surface-exposed, variable loops of the receptor-binding subunit of the ENV scaffold (the surface, or SU, subunit). Structural prediction (e.g. AlphaFold) is used to pre-screen the chimeric sequences for proper folding of the ENV scaffold and exposure of the inserted ligand on its apical surface. At this step, ENVs drawn from multiple MBRACE structural classes can also be tested in silico, to first identify candidate scaffolds that are structurally most compatible with the inserted ligand. Once one or more candidates are identified, the contiguous scaffold+ligand amino-acid sequence is synthesized and cloned into a standard eukaryotic expression vector. The candidate(s) are then compared to the starting scaffold ENV(s) by transfection, SDS-PAGE and immunoblot, to ensure that expression levels and post-translational modifications are comparable to the parental ENV. Candidates are functionally profiled for fusogenicity (by cell-cell fusion assay) and infectivity (using single-cycle, pseudotyped virions). The former is also used to determine whether fusion is pH-dependent, while the latter also tests for efficient transduction of a reporter gene, such as GFP or luciferase. Receptor-positive and receptor-null cells are used to confirm receptor-specific fusion and entry.

Notes: * in-frame ligand can be any peptide/polypeptide sequence that binds a receptor of interest, including (but not limited to) natural or predicted peptide ligands, peptides derived by selection or screening (e.g. by phage-display), heterologous receptor binding domains, DARPins, and receptor-specific antibody-derived sequences, such as single-chain Fv (scFv).

### Engineered Delivery Vehicle

In one aspect, embodiments disclosed herein relate to polynucleotides encoding betaretroviral elements, betaretroviral envelope proteins, or recombinant betaretroviruses, or recombinant betaretroviral envelope proteins for forming an engineered delivery vehicle. In one aspect, embodiments disclosed herein relate to betaretroviral vectors comprising betaretroviral elements, betaretroviral envelope proteins, or recombinant betaretroviruses, or recombinant betaretroviral envelope proteins for forming an engineered delivery vehicle. In another aspect, embodiments disclosed herein are directed to use of such polynucleotides in methods of loading and/or packaging desired cargo molecules. In another aspect, embodiment disclosed herein are directed to such cargo carrying delivery vehicles and methods of using said engineered delivery vehicle to deliver cargo molecules to target cells and tissues.

In some embodiments, the engineered delivery vehicle disclosed herein comprises polynucleotides that encode one or more betaretroviral elements, betaretroviral ENV proteins, or recombinant betaretroviruses for forming an engineered delivery vehicle for packaging cargo or cargo molecules within the delivery vehicle. The polynucleotide may further include regulatory elements such as promoters, enhancers, repressors, inducers, internal ribosome entry site (IRES) to control expression of the vehicle forming system. The polynucleotides are designed for delivery to a cell, a cellular system, and/or tissue to allow expression of the delivery system components and formation of said delivery vehicles including packaging of desired cargo molecules into said delivery vehicles.

In some embodiments, the one or more betaretroviral elements or recombinant betaretroviral envelope proteins for forming a delivery vehicle comprise a betaretroviral envelope protein. The envelope protein further includes one or more receptor binding domain, which is capable of specifically binding to a target cell.

The engineered delivery vehicle may further comprise cargo domain elements, such as transgene encoding a therapeutic protein, and the therapeutic protein comprises an immune checkpoint modulator, an antibody or portion thereof, a fusion protein, an anticoagulant, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, an antiviral, and any combination thereof.

In some embodiments, the engineered delivery vehicle may include regulatory element(s) that control expression of the vehicle-forming system.

In some embodiments, the engineered delivery vehicle within the scope of the present invention may be provided in any form, including but not limited to solid, semi-solid, emulsion, or colloidal particles. As such, any of the delivery systems described herein, including but not limited to, e.g., lipid-based systems, liposomes, micelles, microvesicles, exosomes, or gene gun may be provided as particle delivery systems within the scope of the present invention.

In some embodiments, recombinant vectors can comprise polynucleotides of the invention in a form suitable for expression of the nucleic acid in a host cell, which means that the recombinant vectors include one or more regulatory elements, which may be selected on the basis of the host cells to be used for expression, that is operatively linked to the nucleic acid sequence to be expressed.

In some embodiments, the disclosed engineered delivery vehicle includes polynucleotides encoding one or more betaretroviral elements, betaretroviral envelope proteins, or recombinant betaretroviruses, or recombinant betaretroviral envelope proteins for forming a delivery vehicle for packaging cargo within the delivery vehicle. In some embodiments, the cargo is a transgene encoding a therapeutic protein. Cargoes that can be delivered in accordance with the systems and methods described herein include, but are not necessarily limited to, biologically active agents, therapeutic agents, imaging agents, and monitoring agents. Representative cargo or cargo molecules may include, but are not limited to, an immune checkpoint modulator, an antibody or portion thereof, a fusion protein, an anticoagulant, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, an antiviral, and any combination thereof. The delivery particles described herein may be used and further comprise a number of different cargo molecules for delivery.

In some embodiments, the cargo or cargo molecules may comprise a therapeutic agent. The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In some embodiments, the cargo is a genetic modulating agent.

In some embodiments, the engineered delivery vehicle further comprises a reverse transcriptase.

In some embodiments, the engineered delivery vehicle further comprises an RNA; a DNA; a single-stranded RNA; or a single-stranded DNA.

In some embodiments, the polynucleotide having nucleotide sequences is at least 80% identical to any one of SEQ ID NO: 1-21.

In some embodiments, the polynucleotide having nucleotide sequences comprises a sequence of any one of SEQ ID NO: 1-21.

In some embodiments, the polynucleotide encoding the rENV protein comprises a nucleotide sequence with at least 80% sequence identity to any one of SEQ ID NO: 1-21 or a portion thereof.

In some embodiments, the polynucleotide encoding the rENV protein comprises a nucleotide sequence encoding an SU domain, or a TM domain or both having at least 80% sequence identity to an SU domain or TM domain of SEQ ID NO: 1-21.

In some embodiments, the polynucleotide encoding the rENV has a nucleotide sequence comprising a sequence of any one of SEQ ID NO: 1-21 or a portion thereof.

In some embodiments, the polynucleotide encoding the one or more rENV proteins comprises a nucleotide sequence encoding an SU domain, or a TM domain or both having at least 80% sequence identity to an SU domain or TM domain of SEQ ID NO: 1-21.

In some embodiments, the rENV protein comprises an SU domain, TM domain, variable domain or scaffold domain of a protein having at least 80% sequence identity to any one of SEQ ID NO: 22-42 or a portion thereof.

In some embodiments, the rENV protein comprise an SU domain, TM domain, variable domain or scaffold domain of a protein having a sequence of any one of SEQ ID NO: 22-42 or a portion thereof.

In some embodiments, the engineered delivery vehicle comprises a polynucleotide encoding the one or more rENV proteins and comprises a nucleotide sequence with at least 80% sequence identity to any one of SEQ ID NO: 1-21 or a portion thereof.

In some embodiments, the engineered delivery vehicle comprises a polynucleotide encoding the one or more rENV proteins and comprises a nucleotide sequence encoding an SU domain, or a TM domain or both having at least 80% sequence identity to an SU domain or TM domain of SEQ ID NO: 1-21.

In some embodiments, the engineered delivery vehicle comprises a polynucleotide encoding the one or more rENV proteins and comprises a nucleotide sequence comprising a sequence of any one of SEQ ID NO: 1-21 or a portion thereof.

In some embodiments, the engineered delivery vehicle further comprises an RNA; a DNA; a single-stranded RNA; a single-stranded DNA.

In some embodiments, the engineered delivery vehicle comprises a rENV protein comprising an SU domain, TM domain, variable domain or scaffold domain of a protein having at least 80% sequence identity to any one of SEQ ID NO: 22-42 or a portion thereof.

In some embodiments, the engineered delivery vehicle comprises a rENV protein comprising an SU domain, TM domain, variable domain or scaffold domain of a protein having a sequence of any one of SEQ ID NO: 22-42 or a portion thereof.

The method of claim 33, wherein rENV protein is from a mouse mammary tumor virus (MMTV), a jaagsiekte sheep retrovirus (JSRV), or an intracisternal a-type particle elements with an envelope (IAPE).

In some embodiments, the rENV protein is encoded by the polynucleotide having nucleotide sequences at least 80% identical to any one of SEQ ID NO: 1-21.

In some embodiments, the rENV protein is encoded by the polynucleotide having nucleotide sequences comprises a sequence of any one of SEQ ID NO: 1-21.

In some embodiments, the rENV protein comprises an SU domain, TM domain, variable domain or scaffold domain of a protein having at least 80% sequence identity to any one of SEQ ID NO: 22-42 or a portion thereof.

In some embodiments, the rENV protein comprises an SU domain, TM domain, variable domain or scaffold domain of a protein having a sequence of any one of SEQ ID NO: 22-42 or a portion thereof.

### Use as Research Tools

Provided are methods of delivering cargo to a target cell and/or target tissue by contacting the target cell and/or the target tissue with an engineered delivery vehicle that comprises polynucleotides encoding betaretroviral elements, betaretroviral envelope protein, a recombinant betaretrovirus, or recombinant betaretroviral envelope protein for packaging cargo within the engineered delivery vehicle. Therefore, the disclosed methods can be utilized as research tools in molecular biology or a related field.

In some embodiments, a method of contacting a cell may comprise, for example, contacting a cell in a culture with a polynucleotide encoding a surface subunit domain of envelope protein, a polynucleotide encoding a recombinant receptor binding domain of envelope protein, a scaffold domain of envelope protein, and/or betaretrovirus transmembrane domain of envelope. In some embodiments, the RBD is a receptor binding ligand, which binds specifically to a receptor on a target cell and/or target tissue.

In some embodiments, contacting a cell comprises adding a polynucleotide encoding at least one betaretrovirus envelope protein surface subunit sequence, at least one betaretrovirus envelope protein receptor binding domain sequence, at least one betaretrovirus envelope protein scaffold domain sequence, at least one betaretrovirus envelope protein transmembrane, a reverse transcriptase, or a composition (e.g., a pharmaceutical composition) to a supernatant of a cell culture (e.g., a cell culture on a tissue culture plate or dish).

In some embodiments, a cell described herein is a cell isolated or derived from a subject. In some embodiments, a cell is a mammalian cell (e.g., a cell isolated or derived from a mammal). In some embodiments, a cell is a human cell. In some embodiments, a cell is isolated or derived from a particular tissue of a subject. In some embodiments, a cell is in vitro. In some embodiments, a cell is ex vivo. In some embodiments, a cell is in vivo, e.g., a cell is within a subject (e.g., within a tissue or organ of a subject). In some embodiments, a cell is a primary cell. In some embodiments, a cell is from a cell line (e.g., an immortalized cell line). In some embodiments, a cell is a cancer cell or an immortalized cell.

### Methods of Therapeutic Treatment

An additional aspect of the present disclosure is a method of treating, preventing, or ameliorating a genetic disease, disorder, or syndrome in a subject. The method comprises administering an effective amount of polynucleotide encoding at least one betaretroviral elements, betaretroviral envelope proteins, or recombinant betaretroviruses, or recombinant betaretroviral envelope proteins for forming an engineered delivery vehicle for packaging cargo within the delivery vehicle to the subject (e.g., patient) in need thereof. The method effectuates the amelioration of at least one symptom of the disease, disorder, or syndrome in the subject. In some embodiments, envelope protein includes a surface subunit domain including a recombinant receptor binding domain and a scaffold domain. The envelope protein also includes a betaretrovirus transmembrane domain. The subunit domain is covalently or non-covalently bound to the transmembrane domain. In some embodiments, the RBD is a receptor binding ligand, which binds specifically to a receptor on a target cell and/or target tissue.

In some embodiments described herein, the method involves administering an effective amount of polynucleotides encoding betaretroviral elements, betaretroviral envelope protein, or a recombinant betaretrovirus, or a recombinant betaretroviral envelope protein to a subject.

In some embodiments described herein, the subject is a human, non-human primate, dog, cat, livestock (e.g., cattle, sheep, pig, goat, horse, donkey, mule, buffalo, oxen, llama, camel, alpaca, cow, ox, reindeer, sheep, water buffalo, yak, et.), poultry (e.g., chicken, turkey, etc.).

In some embodiments described herein, the method involves determining whether cells or tissues of the subject express the receptor of interest. The step of determining whether cells or tissues of the subject express the receptor of interest can be performed by any assay or method for detecting a DNA or RNA sequence. The assays or methods include, but are not limited to, genotyping, genome sequencing, next generation sequencing, gene expression analysis, immunohistochemistry, Southern blotting, Western blotting, and polymerase chain reaction (PCR)-based amplification, reverse transcription polymerase chain reaction, real-time reverse transcription polymerase chain reaction (RT-PCR), polymerase chain reaction-restriction fragment length polymorphism (PCR-RFLP), polymerase chain reaction-single strand conformation polymorphism (PCR-SSCP), mutant allele-specific PGR amplification (MASA) assays, oligonucleotide ligation assays, hybridization assays, TaqMan assays, and microarray analyses.

In some embodiments, the envelope protein comprises a cargo-binding domain. The cargo or cargo molecule is a transgene encoding a therapeutic protein, which includes but is not limited to an immune checkpoint modulator, an antibody or portion thereof, a fusion protein, an anticoagulant, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, an antiviral, and any combination thereof.

In some embodiments, the cargo is delivered in the form of virus-like particle.

In some embodiments, disclosed herein is a method of treating a subject with hypercholesterolemia or disorders associated with mutations in low-density lipoprotein (LDL) receptors.

In some embodiments, disclosed herein is a method of treating a subject has a genetic disease, disorder, or syndrome. The disease, disorder, or syndrome includes but is not limited to Cystic Fibrosis, Sickle Cell Anemia, Down Syndrome (Trisomy 21), Huntington's Disease, Duchenne Muscular Dystrophy, Hemophilia, Tay-Sachs Disease, Marfan Syndrome, Fragile X Syndrome, Phenylketonuria, Turner Syndrome, Klinefelter Syndrome, Albinism, Neurofibromatosis, Angelman Syndrome, Prader-Willi Syndrome, Williams Syndrome, Rett Syndrome, Wilson's Disease, Spinal Muscular Atrophy, Xeroderma Pigmentosum, Beckwith-Wiedemann Syndrome, Noonan Syndrome, Leigh Syndrome, Ehlers-Danlos Syndrome.

In some embodiments, disclosed herein is a method of treating cancer, especially those associated with genetic mutations, in a subject. The cancer can be acidophil carcinoma, acinar cell carcinoma, acral lentiginous melanomas, acute granulocytic leukemia, acute lymphoblastic leukemia, acute lymphocytic leukemia, acute myelogenous leukemia, acute myeloid leukemia, adenocarcinoma, adenoid cystic carcinoma, adenomatoid tumor, adenosquamous carcinoma, adrenal carcinoma, adrenal cortex carcinoma, adrenal cortical carcinoma, alveolar (bronchiolar) carcinoma, alveolar rhabdomyosarcoma, amelanotic melanoma, ameloblastic fibrosarcoma, ameloblastic odontosarcoma, ameloblastoma, ampullary carcinoma, androblastoma, angioma, angiosarcoma, apocrine adenocarcinoma, astroblastoma, astrocytoma, basal cell carcinoma, basophil carcinoma, basophilic leukemia, B-cell lymphoma, benign chondroma, bladder cancer, bladder carcinoma, botryoid sarcoma, embryonal rhabdomyosarcoma, brain or spinal cord cancer, branchiolo-alveolar adenocarcinoma, breast cancer, breast carcinoma, brenner tumor, bronchial adenoma, bronchogenic carcinoma, carcinoid tumors, carcinosarcoma, central nervous system cancer, cerebellar sarcoma, ceruminous adenocarcinoma, cervical carcinoma, cervical hyperplasia, cholangiocarcinoma, osteogenic sarcoma, osteosarcoma, chondroblastoma, chondromatous hamartoma, chondromyxofibroma, chondrosarcoma, chordoma, choriocarcinoma, chromophobe carcinoma, chronic granulocytic leukemia, chronic lymphocytic leukemia, chronic myeloblastic leukemia, chronic myelogenous leukemia, clear cell adenocarcinoma, colon cancer, colon carcinoma, colorectal cancer, congenital tumors, cystadenocarcinoma, dermatofibroma, dysgerminoma, embryonal carcinoma, endocrine cancer, endometrial carcinoma, endometroid carcinoma, cosinophilic leukemia, ependymoma, epithelioid cell melanoma, erythroleukemia, esophageal carcinoma, essential thrombocytosis, ewing's sarcoma, extra-mammary paraganglioma, eye cancer, fibrillary astrocytoma, fibroadenoma, fibroma, fibrosarcoma, fibrous histiocytoma, follicular adenocarcinoma, ganglioneuroblastoma, gastrinoma, genitourinary cancer, genitourinary carcinoma, germinoma, pincaloma, giant and spindle cell carcinoma, giant cell tumor of bone, glioblastoma, glioma, glomangiosarcoma, glucagonoma, granular cell carcinoma, granular cell tumor, granuloma, granulosa cell tumor, granulosa-thecal cell tumor, hairy cell leukemia, hamartoma, head-neck cancer, hemangioendothelioma, hemangioma, gall bladder carcinoma, hemangiopericytoma, hemangiosarcoma, hematopoietic cancer, hepatoblastoma, hepatocellular adenoma, hepatocellular carcinoma, infiltrating duct carcinoma, inflammatory carcinoma, insulinoma, interstitial cell carcinoma, intraepithelial carcinoma, juxtacortical osteosarcoma, Kaposi's sarcoma, keloids, kidney cancer, leiomyoma, leiomyosarcoma, melanoma, leukemia, leydig cell tumor, lipid cell tumor, lipoma, hepatoma, liposarcoma, liver cancer, lobular carcinoma, lung carcinoma, lung cancer, lymphangiosarcoma, lymphoepithelial carcinoma, lymphoid leukemia, lymphoma, lymphosarcoma cell leukemia, malignant carcinoid carcinoma, malignant fibrous histiocytoma, malignant giant cell tumor chordoma, malignant histiocytosis, malignant hypercalcemia, malignant lymphoma, malignant melanoma, malignant pancreatic insulinoma, malignant teratoma, malignant tissue, mantle cell lymphoma, mast cell leukemia, mast cell sarcoma, medullary carcinoma, medulloblastoma, megakaryoblastic leukemia, meningioma, meningiosarcoma, mesenchymal chondrosarcoma, mesenchymoma, mesonephroma, mesothelioma, metastatic colorectal cancer, mucinous adenocarcinoma, mucinous cystadenocarcinoma, mucoepidermoid carcinoma, mullerian mixed tumor, multiple myeloma, mycosis fimgoides, mycosis fungoides, myeloid leukemia, myeloid sarcoma, myeloma, myxoma, myxosarcoma, nephroblastoma, neuroblastoma, neurofibroma, neurofibrosarcoma, nodular melanomas, nonencapsulating sclerosing carcinoma, non-Hodgkin's lymphoma, non-small cell lung cancer (NSCLC), odontogenic tumor, olfactory neurogenic tumor, oligodendroblastoma, oligodendroglioma, osteitis deformans, osteochronfroma, osteocartilaginous exostoses, non-hodgkin's lymphomas, ovarian cancer, ovarian carcinoma, ovarian stromal tumor, oxyphilic adenocarcinoma, ductal adenocarcinoma, pancreatic cancer, pancreatic carcinoma, papillary adenocarcinoma, papillary and follicular adenocarcinoma, papillary carcinoma, papillary cystadenocarcinoma, papillary serous cystadenocarcinoma, papillary transitional cell carcinoma, paragranuloma, pheochromocytoma, phyllodes tumor, pilomatrix carcinoma, placental cancer, plasma cell leukemia, polycythemia vera, pre-tumor cervical dysplasia, primary brain carcinoma, primary macroglobulinemia, prostate cancer, prostatic carcinoma, protoplasmic astrocytoma, neuroblastoma, rectal cancer, renal cell carcinoma, retinoblastoma, rhabdomyoma, rhabdomyosarcoma, sarcoma, schwannoma, sebaceous adenocarcinoma, sertoli cell carcinoma, Sertoli-Leydig cell tumors, signet ring cell carcinoma, skin appendage carcinoma, skin cancer, small cell carcinoma, small cell lung cancer (SCLC), small lymphocytic (SL) NHL, soft tissue cancer, soft-tissue sarcoma, solid carcinoma, spinal cord neurofibroma, squamous cell carcinoma, stomach carcinoma, stomach cancer, stromal sarcoma, superficial spreading melanoma, synovial sarcoma, teratocarcinoma, teratoma, testicular carcinoma, the cancer is colon cancer, thecoma, thymoma, thyroid carcinoma, trabecular adenocarcinoma, transitional cell carcinoma, tubular adenoma, villous adenoma, Wilm's tumor, xanthoma or a combination thereof.

In some embodiments, the polynucleotides encoding betaretroviral elements, betaretroviral envelope protein, or a recombinant betaretrovirus, or a recombinant betaretroviral envelope protein disclosed herein are used as a gene therapy to transport functional copies of genes, RNA molecules, or gene-editing components to subjects, to modify, replace or introduce genetic materials into the subject's cell to treat or prevent genetic disease, disorder, or syndrome.

In some embodiments, the polynucleotides encoding betaretroviral elements, betaretroviral envelope protein, or a recombinant betaretrovirus, or a recombinant betaretroviral envelope protein disclosed herein are used as a delivery vehicle for packaging cargo within the delivery vehicle to the subject (e.g., patient) in need thereof.

In some embodiments, the polynucleotides encoding betaretroviral elements, betaretroviral envelope protein, or a recombinant betaretrovirus, or a recombinant betaretroviral envelope protein disclosed herein can be administered via any suitable route, which may depend on, e.g., the medical condition being treated and its location and the pharmacokinetics of the inhibitor. Potential routes of administration include without limitation oral, parenteral (including intradermal, subcutaneous, intramuscular, intravascular, intravenous, intra-arterial, intraperitoneal, intracavitary, intramedullary, intrathecal and topical), and topical (including dermal/epicutaneous, transdermal, mucosal, transmucosal, intranasal [e.g., by nasal spray or drop], ocular/intraocular [e.g., by eye drop], pulmonary [e.g., by oral or nasal inhalation], buccal, sublingual, rectal [e.g., by suppository], and vaginal [e.g., by suppository]).

In some embodiments, polynucleotides encoding betaretroviral elements, betaretroviral envelope protein, or a recombinant betaretrovirus, or a recombinant betaretroviral envelope protein are administered at an effective amount to a subject once. In some embodiments, polynucleotides encoding betaretroviral elements, betaretroviral envelope protein, or a recombinant betaretrovirus, or a recombinant betaretroviral envelope protein are administered at an effective amount to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a genetic disease, disorder, or condition in the subject.

Administration of the delivery vehicle to the subject may involve suitable carriers, excipients, and other agents within formulations that enhance transfer, delivery, tolerance, and similar factors. Numerous appropriate formulations are detailed in a standard reference for pharmaceutical chemists: Remington's Pharmaceutical Sciences (15th ed., Mack Publishing Company, Easton, Pa. (1975)), specifically in Chapter 87 by Seymour Blaug. Examples of such formulations include powders, pastes, ointments, jellies, waxes, oils, lipid-based vesicles (cationic or anionic) such as Lipofectin™, DNA conjugates, anhydrous absorption pastes, oil-in-water and water-in-oil emulsions, carbowax (polyethylene glycols of various molecular weights), semi-solid gels, and mixtures containing carbowax. Any of these formulations may be suitable for use in treatments and therapies under this invention, provided the active ingredient remains active within the formulation and is physiologically compatible and tolerable for the chosen administration route.

Carriers include excipients and diluents and must be of sufficiently high purity and sufficiently low toxicity to render them suitable for administration to the patient being treated.

The carrier can be inert, or it can possess pharmaceutical benefits of its own. The amount of carrier employed in conjunction with the compound is sufficient to provide a practical quantity of material for administration per unit dose of the compound. Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

In some embodiments, the engineered delivery vehicle disclosed herein may be delivered to a subject together with a liposome. Suitable lipids for liposomal formulation include, without limitation, monoglycerides, diglycerides, sulfatides, lysolecithin, phospholipids, and the like.

In some embodiments, a delayed or sustained release of polynucleotides encoding betaretroviral elements, betaretroviral envelope protein, or a recombinant betaretrovirus, or a recombinant betaretroviral envelope protein for forming an engineered delivery vehicle for packaging cargo within the delivery vehicle can also be formulated as, e.g., a depot that can be implanted in or injected into a subject, e.g., intramuscularly, intracutaneously or subcutaneously. A depot formulation can be designed to deliver the nucleic acid over an extended period of time, e.g., over a period of at least about 1 week, 2 weeks, 3 weeks, 1 month or 3 months. For example, the nucleic acid can be formulated with a polymeric material (e.g., polyethylene glycol [PEG], polylactic acid [PLA] or polyglycolic acid [PGA], or a copolymer thereof [e.g., PLGA or PLA-PEG]), with a hydrophobic material (e.g., as an emulsion in an oil) and/or an ion-exchange resin, as a more lipophilic derivative (e.g., as an ester of or a salt with a fatty acid such as a C8-C20 fatty acid [e.g., decanoic acid]), or as a sparingly soluble derivative (e.g., a sparingly soluble salt).

In some embodiments, polynucleotides encoding betaretroviral elements, betaretroviral envelope protein, or a recombinant betaretrovirus, or a recombinant betaretroviral envelope protein for forming a delivery vehicle for packaging cargo within the delivery vehicle can also be contained or dispersed in a matrix material. The matrix material can comprise a polymer (e.g., ethylene-vinyl acetate) and controls the release of the compound by controlling dissolution and/or diffusion of the compound from, e.g., a reservoir, and can enhance the stability of the compound while contained in the reservoir. Such a release system can be designed as a sustained-release system, can be configured as, e.g., a transdermal or transmucosal patch, a microneedle transdermal patch, and can contain an excipient that can accelerate the compound's release, such as a water-swellable material (e.g., a hydrogel) that aids in expelling the compound out of the reservoir.

### Examples

The following examples are intended only to illustrate the disclosure. Other procedures, methodologies, techniques, reagents and conditions may alternatively be used as appropriate.

Presented are exemplary ENVs. There are likely thousands of available betaretroviral ENV proteins available in mammalian genomes for integration in the compositions and methods described herein. As the library of characterized ENVs grows, starting scaffolds for optimization are chosen based on structural similarity to the ligand of interest or in silico predicted “best fit” between a ligand and scaffold. Also, as the cognate receptors for these ENVs are identified, they may also prove to be useful without further modification.

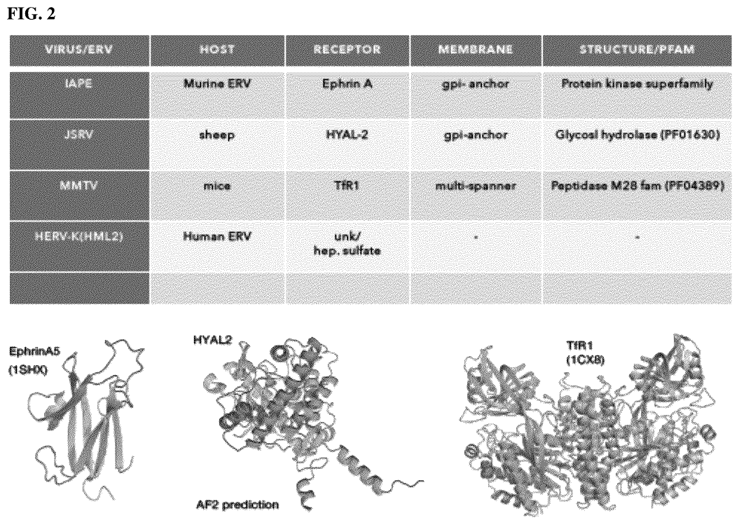

We took note of the fact that three divergent betaretrovirus ENVs, from mouse mammary tumor virus (MMTV), jaagsiekte sheep retrovirus (JSRV), and “intracisternal a-type particle elements with an envelope” (or IAPE), use unrelated cell-surface receptors representing different protein families (FIG. 2). Thus, murine transferrin receptor-1 (mTfR1) is the entry receptor for MMTV, hyalouronidase-2 (HYAL-2) is the receptor for JSRV, and Ephrin A1-A5 are receptors for mouse IAPEs 23. While a receptor for “type-D” betaretroviruses is known, these are natural recombinants that have acquired a novel gammaretrovirus env, and type-D entry is identical to that of gammaretroviruses 4.5. To date, a receptor for human endogenous retroviruses in the HML2 group (HERV-K (HML2)) has not been identified, but HML2 ENV does not use any of the three known receptors. Taken together, these observations suggest that betaretrovirus ENV proteins are inherently adaptable, having evolved over deep time to use divergent cell-surface proteins as entry receptors.

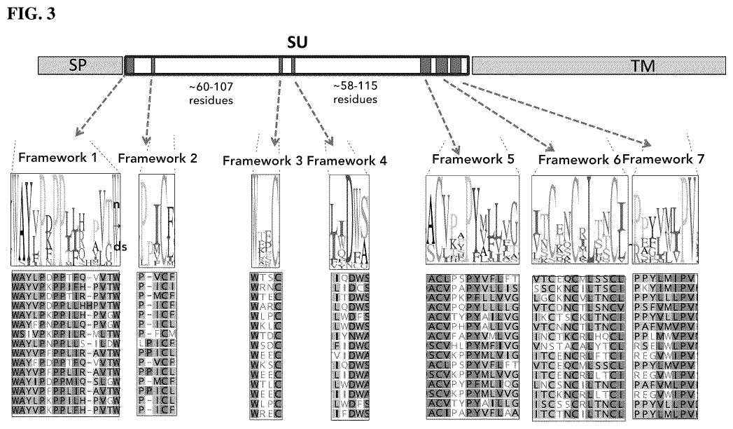

At present, there are no high-resolution structures of a betaretrovirus ENV. There is one report describing loss-of-function mutations in the MMTV ENV 6, pointing to a region of SU that may be involved in receptor binding. A second study based on JSRV and ENTV ENV chimeras identified residues that may explain differential affinity for HYAL-27. These studies indicate approximate regions of SU that may affect receptor-specificity but lack the resolution to define receptor binding domains (RBDs) and their functional intermolecular interactions within the ENV complex. Investigating the relationship between Env structure and receptor-recognition for betaretroviruses is limited by this lack of structural insight. To overcome this limitation, we exploited the vast genomic “archive” of betaretrovirus sequences present as endogenous retrovirus (ERV) loci in mammalian genomes to create an evolution-guided, working model of a canonical betaretrovirus SU domain. Briefly, we used iterative PHI-BLAST and a short, conserved seed pattern (XWAYXPXPPX, where X=any amino-acid) identified from an alignment of the MMTV, JSRV and HML2 SUs to identify ERV loci with intact betaretrovirus-related Env ORFs. When a candidate ORF was identified, surrounding sequences (˜10 kB 5′ and 3′ to the hit) were extracted to identify additional proviral elements and to confirm taxonomic relationships to the Betaretroviruses. Together, these ERV ORFs represent hundreds of millions of years of betaretrovirus evolutionary history. We next used amino acid sequence alignments and secondary structure prediction to identify conserved and variable regions in the linear sequences. We focused specifically on the putative SU regions, where the major receptor-binding elements are likely to be located. By sequence inspection, we noted conservation of multiple, short sequence stretches in SU, interspersed between stretches of considerable sequence and length variation (FIG. 3).

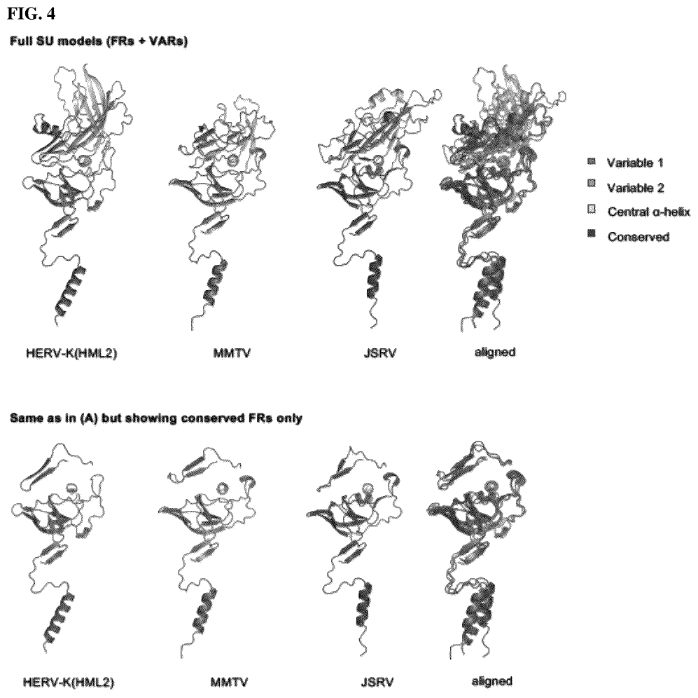

Next, we used the artificial intelligence program AlphaFold to generate three-dimensional structural models of each of the input SU protein sequences. Alphafold predicts that all the betaretrovirus SU sequences examined fold into structures consisting of two domains, one comprising the FR sequences and the other comprising the VAR sequences. The FR domain takes on a similar “chalice-shaped” structure for all the SUs analyzed, while the VAR domain structures differ significantly (FIG. 4). In all cases, a conserved alpha-helix is predicted to be within the FR-VAR interface, perpendicular to the long axis of SU. Similar predictions were recently reported for SU of MMTV, JSRV and HML2⁸.

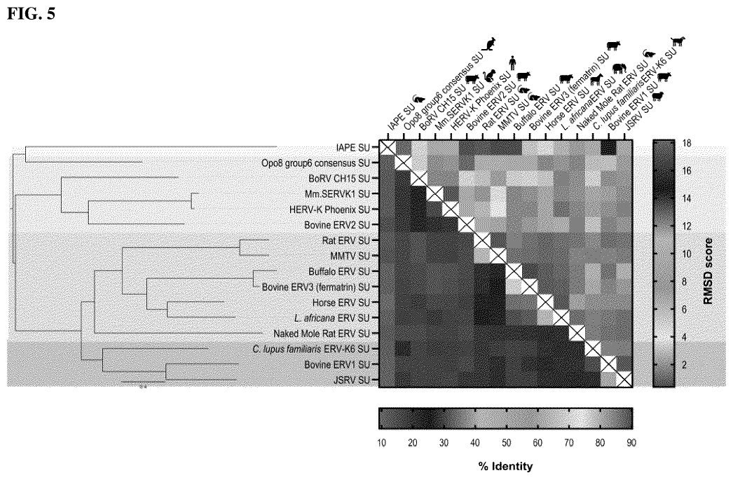

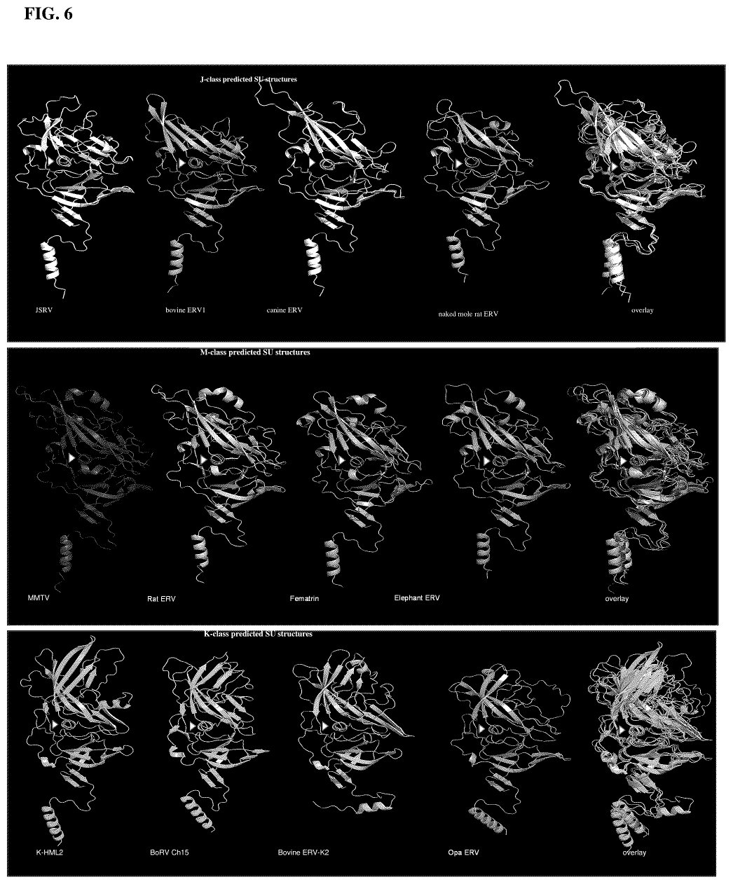

Phylogenetic clustering based on amino-acid alignments and structural clustering based on RMSD values of superimposed structures both suggest that the betaretrovirus SU domains comprise three classes (FIG. 5). Conveniently, each of these proposed classes includes a well-studied, functional example—thus, we refer to these as the K-class (with HERV-K (HML2) as the prototype), the J-class (JSRV is the prototype) and M-class (MMTV is the prototype) (FIG. 6). These three viruses use different receptors, consistent with the hypothesis that structural variation reflects adaptation to different receptor proteins. A potential fourth class is represented by the structure of the IAPE-D ENV (I-class), which retains the chalice-shaped FR domain but has a divergent VAR domain.

Based on these models, we propose that VARs comprise the unique receptor binding domains (RBD) of each class, while the FRs fold into a conserved “scaffold” common to all betaretrovirus ENVs. The contrast between structurally superimposable scaffolds and dissimilar VAR domains likely reflects millions of years of diversifying selection on the VAR domain (e.g., adaptation to different receptors), while the essential, receptor-independent functions of the FR domain are constrained and subject to purifying selection (e.g., assembly into heterotrimeric spikes, incorporation into virions, and membrane fusion). In this scenario, separate folding of the two linked domains may have provided the structural flexibility that allowed receptor-switching and host-switching events during co-evolution of betaretroviruses and their mammalian hosts, which in turn contributed to the broad distribution of betaretroviruses and betaretroviral ERVs among modern mammals (including humans).

To our knowledge, endogenous retrovirus (ERV) sequences have not previously been exploited as a source of entry proteins for gene therapy applications. We have already identified, synthesized and characterized a small panel of divergent betaretroviral ENV proteins among ERV loci in vertebrate genome assemblies, and confirmed that several of these can be expressed in cell culture, and produce proteins which behave as predicted for betaretrovirus ENV proteins (FIG. 8). ERV loci with bioinformatically predicted ORFs encoding putative betaretrovirus ENV proteins were used to generate synthetic expression constructs, including an in-frame, C-terminal Avi-tag. These were assessed for expression by transient transfection and western blot of cell lysates. FIG. 8 depicts several such ENVs. Lysates in lanes with a “+” were pre-treated with PNGaseF to remove mature glycans. The blots confirm that all but one of these are expressed at the predicted sizes for the full length, glycosylated precursors (SU-TM), and that they are furin cleaved to produce TM subunits of the predicted size. One, Bovine ERV1, was expressed but not cleaved, and site-directed mutation to correct the furin cleavage site resulted in an unstable or poorly expressed protein. With this exception, expression and processing are consistent with expectations for betaretrovirus ENV proteins. (IAPE-D1, mouse ERV; BoRV CH15, bovine betaretrovirus CH15; SERV-K1, rhesus macaque ERV-K; HERV-K, human ERV-K; MMTV, mouse betaretrovirus; BoERV3, bovine ERV 3; BoERV1, bovine ERV-1; BoERV1 L382R, bovine ERV-1 with a modified furin cleavage site).

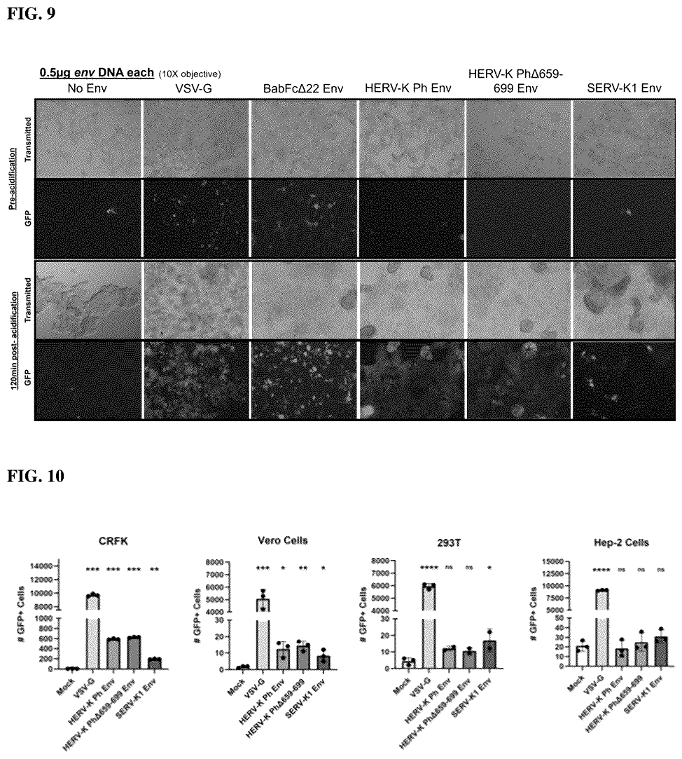

To be useful for viral vectors, these ENVs must be capable of directing membrane fusion, which we measure using a cell-cell fusion assay and a “split” green fluorescent protein (GFP) reporter system (FIG. 9). Cell-cell fusion mediated by endogenous retrovirus encoded ENVs. Two cell lines stably expressing different halves of a split GFP are mixed and transfected with ENV expression constructs. If the ENV mediates cell-cell fusion, GFP multimers form and fluoresce, usually within minutes of cell fusion. Cells were either left at neutral pH (upper two rows) or pulsed with low pH (lower two rows). “No Env” was an empty vector that served as a negative control, and VSV-G served as a positive control. BabFCdelta22 ENV is an ERV-encoded ENV from the baboon genome with a truncated cytoplasmic domain that enhances fusogenicity; HERV-K Ph is a previously reported human ERV ENV, HERV-K (HML2); HERV-K Phdelta659-699 is the same ENV with a truncation of the cytoplasmic domain that increases fusogenicity. SERV-K1 ENV is a betaretrovirus ENV encoded by an ERV in the rhesus macaque genome. As expected, fusion by the three betaretrovirus ENVs was pH dependent (last three columns, compare upper and lower panels).

We also screen candidate ENVs using a lentivirus vector system, which produces lentivirus particles that package the gene for green fluorescent protein (GFP). This assay confirms that the synthetic ENV is compatible with a lentivirus vector system, and that the combination can be used to deliver a genetic “payload” (i.e., a GFP reporter gene) (FIG. 10). Lentivirus vectors can be pseudotyped with ERV-derived betaretrovirus ENVs and deliver a reporter to feline, monkey and human cell lines (CRFK, Vero and 293T cells, respectively). 293T cells were cotransfected with a lentiviral vector encoding the viral structural proteins and a transducible GFP reporter and an ENV expression construct. Supernatants were harvested 48-72 hours post-transfection and used to infect the indicated cell lines. VSV-G pseudotyped vector was used as a positive control. Hep-2 cells are known to be refractory to HERV-K mediated infection. Dots indicate biological replicates. * significantly different from mock/background (p<0.01, 0.001, <0.0001, ns=not significant).

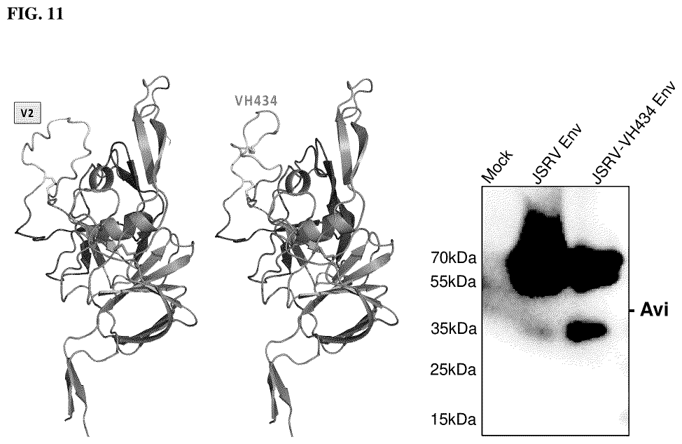

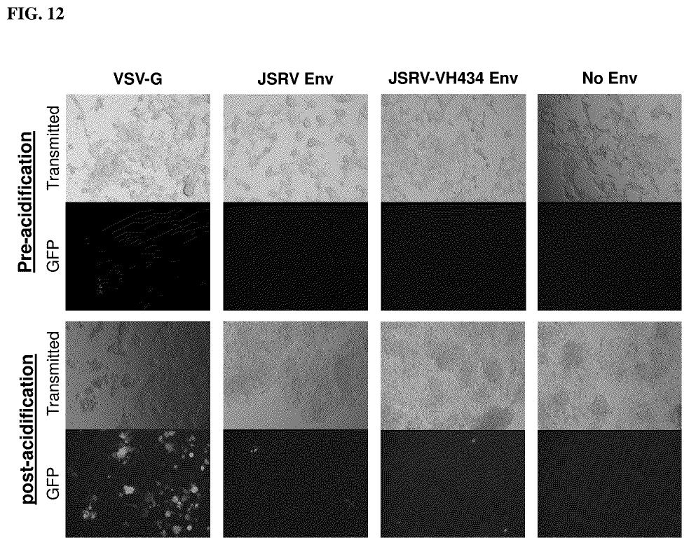

One approach to redirecting the receptor specificity of a betaretrovirus ENV is to modify the apical surface (the receptor binding surface) of the ENV SU subunit to display a synthetic peptide ligand for a known receptor. AI-based methods for structure prediction can be used for in silico screening of modified ENVs displaying such peptides, followed by experimental confirmation and iterative redesign (FIGS. 11 and 12). FIG. 11 shows the expression and processing of a betaretrovirus ENV engineered to encode a peptide ligand. AlphaFold was used to predict structures of the JSRV ENV SU subunit wild type (left) or with insertions in the 2nd variable domain (V2) of a cyclic 8-residue peptide (VH434, blue and yellow). VH434 is reported to be a ligand for the human LDL receptor (David, M. et al. 2018. PLOS One. 13, e0191052). A version predicted to result in minimal divergence from the parental structure was identified by in silico screening (middle). This sequence was then synthesized and expressed by transient transfection and visualized by SDS-PAGE and western blot (right). The synthetic ENV produces a precursor protein of the expected size (upper band) and a lower band corresponding to the furin-released TM subunit. FIG. 12 shows the wild type JSRV ENVwt and JSRV ENVVH434 display similar, low level, pH-dependent fusion in human cells. Upper panels are at neutral pH, lower panels are after a brief, low pH (5.0) pulse. Small syncytia and GFP+ cells indicate membrane fusion. An empty vector served as a negative control (“No Env”). VSV-G served as a positive control. JSRV is an ovine betaretrovirus, and low-level fusion results from suboptimal binding to the human ortholog of the HYAL2 receptor.

These pilot experiments confirm that ERV ORFs found in vertebrate genomes and predicted to encode betaretrovirus ENV proteins can be used to create synthetic ENV-expression constructs. The predicted ENV proteins can be expressed in transfected cells, screened in cell-cell fusion assays for fusogenicity, and used to deliver a heterologous gene to cells using pseudotyped lentivirus particles (FIGS. 8, 9 and 10). AI prediction (AlphaFold) can be used to assist in the design and in silico screening of modified versions of these betaretrovirus ENVs, including ENVs with SU domains that display short peptide ligands for heterologous cell-surface receptors (FIGS. 11 and 12). These tools and results indicate that it is feasible to design, produce and test betaretrovirus ENV proteins that have been customized for binding to pre-specified cell surface receptors. These can then be optimized and integrated with existing lentiviral vector platforms for gene-delivery and gene-therapy applications.

### REFERENCES

The following references are incorporated herein by reference in their entirety for all purposes.
1 Hunter, E. in Retroviruses (eds J. M. Coffin, S. H. Hughes, & H. E. Varmus) (1997).
2 Golovkina, T. V. et al. A novel membrane protein is a mouse mammary tumor virus receptor. J Virol 72, 3066-3071 (1998).
3 Rai, S. K., Duh, F. M., Vigdorovich, V., Danilkovitch-Miagkova, A., Lerman, M. I, Miller, A. D. Candidate tumor suppressor HYAL2 is a glycosylphosphatidylinositol (GPI)-anchored cell-surface receptor for jaagsiekte sheep retrovirus, the envelope protein of which mediates oncogenic transformation. PNAS 8, 4443-4448 (2001).
4 Kewalramani, V. N., Panganiban, A. T. & Emerman, M. Spleen necrosis virus, an avian immunosuppressive retrovirus, shares a receptor with the type D simian retroviruses. J Virol 66, 3026-3031 (1992).
5 Tailor, C. S., Nouri, A., Zhao, Y., Takeuchi, Y. & Kabat, D. A sodium-dependent neutral-amino-acid transporter mediates infections of feline and baboon endogenous retroviruses and simian type D retroviruses. J Virol 73, 4470-4474 (1999).
6 Zhang, Y., Rassa, J. C., deObaldia, M. E., Albritton, L. M. & Ross, S. R. Identification of the receptor binding domain of the mouse mammary tumor virus envelope protein. J Virol 77, 10468-10478 (2003).
7 Dirks, C., Duh, F. M., Rai, S. K., Lerman, M. I. & Miller, A. D. Mechanism of cell entry and transformation by enzootic nasal tumor virus. J Virol 76, 2141-2149 (2002).
8 Hotzel, I. Domain Organization of Lentiviral and Betaretroviral Surface Envelope Glycoproteins Modeled with AlphaFold. J Virol 96, e0134821 (2022).

What is claimed is:

1. A nucleic acid comprising a polynucleotide encoding a transcription unit for a recombinant betaretroviral envelope (rENV) protein comprising a receptor binding domain (RBD), a betaretrovirus surface subunit (SU) domain, and a betaretrovirus transmembrane (TM) domain.

2. The nucleic acid of claim 1, wherein the RBD is a receptor binding ligand (RBL), wherein the RBL is a peptide, polypeptide or protein ligand binding specifically to a receptor on a cell, a tissue and/or a mammal, and wherein the cell is a mammalian cell, the tissue is a mammalian tissue, and wherein the mammal is a human.

3. The nucleic acid of claim 1, wherein the nucleic acid comprises another polynucleotide encoding a transcription unit for a therapeutic protein for treating or preventing a disease, disorder or condition.

4. The nucleic acid of claim 3, wherein the therapeutic protein comprises at least one of an immune checkpoint modulator, an antibody or portion thereof, a fusion protein, an anticoagulant, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, or an antiviral.

5. The nucleic acid of claim 1, wherein rENV protein is from a mouse mammary tumor virus (MMTV), a jaagsiekte sheep retrovirus (JSRV), or an intracisternal a-type particle elements with an envelope (IAPE).

6. The nucleic acid of claim 1, wherein the polynucleotide encoding the rENV protein comprises a nucleotide sequence with at least 80% sequence identity to any one of SEQ ID NO: 1-21 or a portion thereof.

7. The nucleic acid of claim 1, wherein polynucleotide encoding the rENV protein comprises a nucleotide sequence encoding an SU domain, or a TM domain or both having at least 80% sequence identity to an SU domain or TM domain of SEQ ID NO: 1-21.

8. The nucleic acid of claim 1, wherein the polynucleotide encoding the rENV has a nucleotide sequence comprising a sequence of any one of SEQ ID NO: 1-21 or a portion thereof.

9. The nucleic acid of claim 1, wherein the rENV protein comprising an SU domain, TM domain, variable domain or scaffold domain of a protein having at least 80% sequence identity to any one of SEQ ID NO: 22-42 or a portion thereof.

10. The nucleic acid of claim 1, wherein the rENV protein comprising an SU domain, TM domain, variable domain or scaffold domain of a protein having a sequence of any one of SEQ ID NO: 22-42 or a portion thereof.

11. An engineered delivery vehicle comprising (i) a polynucleotide encoding one or more recombinant betaretroviral envelope (rENV) proteins including a receptor binding domain (RBD), a betaretrovirus surface subunit (SU) domain, and a betaretrovirus transmembrane (TM) domain, (ii) a rENV protein including a receptor binding domain (RBD), a betaretrovirus surface subunit (SU) domain, and a betaretrovirus transmembrane (TM) domain, or (iii) both.

12. The engineered delivery vehicle of claim 11, wherein the RBD is a receptor binding ligand (RBL), wherein the RBL is a peptide, polypeptide or protein ligand binding specifically to a receptor on a cell, a tissue and/or a mammal, and wherein the cell is a mammalian cell, the tissue is a mammalian tissue, and wherein the mammal is a human.

13. The engineered delivery vehicle of claim 11, wherein rENV protein is from a mouse mammary tumor virus (MMTV), a jaagsiekte sheep retrovirus (JSRV), or an intracisternal a-type particle elements with an envelope (IAPE).

14. The engineered delivery vehicle of claim 11, wherein the polynucleotide encoding the one or more rENV proteins comprises a nucleotide sequence with at least 80% sequence identity to any one of SEQ ID NO: 1-21 or a portion thereof.

15. The engineered delivery vehicle of claim 11, wherein the polynucleotide encoding the one or more rENV proteins comprises a nucleotide sequence encoding an SU domain, or a TM domain or both having at least 80% sequence identity to an SU domain or TM domain of SEQ ID NO: 1-21.

16. The engineered delivery vehicle of claim 11, wherein the polynucleotide encoding the one or more rENV proteins comprises a nucleotide sequence comprising a sequence of any one of SEQ ID NO: 1-21 or a portion thereof.

17. The engineered delivery vehicle of claim 11, further comprising an RNA; a DNA; a single-stranded RNA; a single-stranded DNA.

18. The engineered delivery vehicle of claim 11, wherein the rENV protein comprising an SU domain, TM domain, variable domain or scaffold domain of a protein having at least 80% sequence identity to any one of SEQ ID NO: 22-42 or a portion thereof.

19. The engineered delivery vehicle of claim 11, wherein the rENV protein comprising an SU domain, TM domain, variable domain or scaffold domain of a protein having a sequence of any one of SEQ ID NO: 22-42 or a portion thereof.

20. An engineered delivery vehicle that comprises a virus-like particle having a recombinant betaretroviral envelope (rENV) protein on its surface, and including a packaged nucleic acid viral vector payload, wherein the rENV protein comprises a receptor binding domain (RBD), a betaretrovirus surface subunit (SU) domain, and a betaretrovirus transmembrane (TM) domain.

21. The engineered delivery vehicle of claim 20, wherein the packaged nucleic acid viral vector payload comprises a polynucleotide including a rENV transcription unit encoding the rENV protein, and a therapeutic protein transcription unit encoding a therapeutic protein.

22. The engineered delivery vehicle of claim 20, wherein the RBD is a receptor binding ligand (RBL).

23. The engineered delivery vehicle of claim 22, wherein the RBL is a peptide, polypeptide or protein ligand binding specifically to a receptor on a cell, and/or a tissue of a mammal.

24. The engineered delivery vehicle of claim 23, wherein the mammalian is a human.

25. The engineered delivery vehicle of claim 21, wherein the therapeutic protein comprises an immune checkpoint modulator, an antibody or portion thereof, a fusion protein, an anticoagulant, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, or an antiviral.

26. The engineered delivery vehicle of claim 20, wherein rENV protein is from a mouse mammary tumor virus (MMTV), a jaagsiekte sheep retrovirus (JSRV), or an intracisternal a-type particle elements with an envelope (IAPE).

27. The engineered delivery vehicle of claim 20, wherein the polynucleotide encoding the one or more rENV proteins comprises a nucleotide sequence with at least 80% sequence identity to any one of SEQ ID NO: 1-21 or a portion thereof.

28. The engineered delivery vehicle of claim 20, wherein the polynucleotide encoding the one or more rENV proteins comprises a nucleotide sequence encoding an SU domain, or a TM domain or both having at least 80% sequence identity to an SU domain or TM domain of SEQ ID NO: 1-21.

29. The engineered delivery vehicle of claim 20, wherein the polynucleotide encoding the one or more rENV proteins comprises a nucleotide sequence comprising a sequence of any one of SEQ ID NO: 1-21 or a portion thereof.

30. The engineered delivery vehicle of claim 20, further comprising an RNA; a DNA;
a single-stranded RNA; a single-stranded DNA.

31. The engineered delivery vehicle of claim 20, wherein the rENV protein comprising an SU domain, TM domain, variable domain or scaffold domain of a protein having at least 80% sequence identity to any one of SEQ ID NO: 22-42 or a portion thereof.

32. The engineered delivery vehicle of claim 20, wherein the rENV protein comprising an SU domain, TM domain, variable domain or scaffold domain of a protein having a sequence of any one of SEQ ID NO: 22-42 or a portion thereof.

33. A method of forming an engineered delivery vehicle comprising the steps of:
contacting a target cell and/or a target tissue with a nucleic acid including (i) a polynucleotide encoding one or more recombinant betaretroviral envelope (rENV) proteins having a receptor binding domain (RBD), a betaretrovirus surface subunit (SU) domain, and a betaretrovirus transmembrane (TM) domain, and (ii) at lease one additional polynucleotide including a transcription unit encoding a protein,
wherein the RBD binds a receptor of interest and wherein expression of the one or more rENV proteins by the target cell and/or the target tissue forms an engineered delivery vehicle including rENV proteins on its surface, and wherein the delivery vehicle includes a payload comprising the at least one additional polynucleotide encoding a protein.

34. A method of delivering a therapeutic payload to a target cell and/or a target tissue comprising the step of administering the engineered delivery vehicle resulting from claim 33.

35. The method of claim 33, further comprising contacting the target cell and/or the target tissue with a reverse transcriptase.

36. The method of claim 33, wherein the engineered delivery vehicle is a virus-like particle.

37. The method of claim 33, wherein the RBD is a receptor binding ligand (RBL), wherein the RBL is a peptide, polypeptide or protein ligand binding specifically to a receptor on a cell, and/or a tissue.

38. The method of claim 37, wherein the cell is a mammalian cell.

39. The method of claim 38, wherein the mammal is a human.

40. The method of claim 33, wherein the protein encoded by the at least one additional polynucleotide including a transcription unit is a therapeutic protein.

41. The method of claim 40, wherein the therapeutic protein comprises an immune checkpoint modulator, an antibody or portion thereof, a fusion protein, an anticoagulant, an immunosuppressive agent, an immunostimulatory agent, an enzyme, a growth factor, a hormone, an interferon, an interleukin, a thrombolytic, an anti-angiogenic, a chemotherapeutic, an antibiotic, an antifungal, or an antiviral.

42. The method of claim 33, wherein rENV protein is from a mouse mammary tumor virus (MMTV), a jaagsiekte sheep retrovirus (JSRV), or an intracisternal a-type particle elements with an envelope (IAPE).

43. The method of claim 33, wherein the rENV protein encoded by the polynucleotide having nucleotide sequences at least 80% identical to any one of SEQ ID NO: 1-21.

44. The method of claim 33, wherein the rENV protein encoded by the polynucleotide having nucleotide sequences comprises a sequence of any one of SEQ ID NO: 1-21.

45. The method of claim 33, wherein the rENV protein comprising an SU domain, TM domain, variable domain or scaffold domain of a protein having at least 80% sequence identity to any one of SEQ ID NO: 22-42 or a portion thereof.

46. The method of claim 33, wherein the rENV protein comprising an SU domain, TM domain, variable domain or scaffold domain of a protein having a sequence of any one of SEQ ID NO: 22-42 or a portion thereof.
